SEQUENCE LISTING (488 sequences)

SEQ ID NO: 1 - protein, 120 aa, Artificial Sequence
  Synthetic: NP3_A3
DVQLQESGPELKKPGETVKISCKASGYTFTDYAIHWVKQAPGKGLKWMGWINTETGEPKYAEDFKGRFAFSLETSVSTAYLQSNNLKNEDTATYFCARHYRYDEAWFAYWGRGTLVTVSS

SEQ ID NO: 2 - protein, 119 aa, Artificial Sequence
  Synthetic: NP3_A4
QVTLKESGPGILKPSQTLSLTCSFSGFSLSTSGMGVGWIRQPSGKGLEWLAHIWWDDDKYHNPSLKSQLTISKDTSRNQVFLKITSVDTADTATYYCARSGYRGNFDYWGQGTTVTVSS

SEQ ID NO: 3 - protein, 120 aa, Artificial Sequence
  Synthetic: NP3_A6
QIQLVQSGPELKKPGETVKIACKASGYTFTDYSMNWVKQAPGKGLKWMGWINTETGEPTYEDDFKGRFAFSLETSASTAYLQITNLKNEDTATYFCVRGYRYDEAWFVYWGQGTLVTVSA

SEQ ID NO: 4 - protein, 117 aa, Artificial Sequence
  Synthetic: NP3_A7
QIQLVQSGAELVKPGASVKLSCTASGFNIKDTYIHWVRQRPEQGLEWIGRIDPANGDTKYDPKFQDKATLIADTSSNTAYLQLGSLTSEDTAVYYCARYRIYYFDYWGQGTTLTVSS

SEQ ID NO: 5 - protein, 122 aa, Artificial Sequence
  Synthetic: NP3_A8
QVQLQQSGAELVKPGASVKLSCKASGYTFTSYYMYWVKQRPGQGLEWIGEINPSNGGFSFNEKFKSKATLTVDKSSSTAYMQLTSLTSEDSAVYYCTRLEGYGDSDYAMIYWGQGTTVTVSS

SEQ ID NO: 6 - protein, 120 aa, Artificial Sequence
  Synthetic: NP3_A9
EVQLVESGAELVKPGASVKISCKASGYTFTDYNMDWVKQSHGKSLEWIGDINPDYGSTSYNQKFRGKATLTVDKSSSTANMELRSLTSEDTAVYYCARGDYDGGYGMDYWGQGTSVTVSS

SEQ ID NO: 7 - protein, 118 aa, Artificial Sequence
  Synthetic: NP3_A10
QVQLQQSGPSLVKPSQTLSFTCSVTGDSVTSGYWNWIRKFPGNKLEFMGYISYSGSTYYSPSLKSRISIIRDTSKNRYYLQLNSVTTEDTATYFCARGLRFFYAMDYWGQGTTLTVSS

SEQ ID NO: 8 - protein, 120 aa, Artificial Sequence
  Synthetic: NP3_A11
EVQLQESGPELKKPGETVKISCKVSGYTFTNYGMNWVKQAPGKGLKWMGWINTYTGEPKYGDDFKGRFALSLETSAGTAYLRINNLKNEDTATYFCARSHGNYENHFDYWGQGTTLTVSS

SEQ ID NO: 9 - protein, 113 aa, Artificial Sequence
  Synthetic: NP3_B1
QVQLQQSGPELVKPGTSVTVYCKASGYTFTTYNIYWVKQSHGKSLEWIGYIDPYNGGTSYNQKFKGKATLTVDKSSSAAYMHLNSLTSEDSAVYYCALPWDYWGQGTTVTVSS

SEQ ID NO: 10 - protein, 119 aa, Artificial Sequence
  Synthetic: NP3_B2
QIQLVQSGGGLVKPGGSLKLSCAASGFTFSDYYMSWVRQTPEKRLEWVATISDGGSYTYYPDSVKGRFTISRDNAKNNLYLHMSSLKSEDTAMYYCARGSTYGNPLDYWGQGTTLTVSS

SEQ ID NO: 11 - protein, 119 aa, Artificial Sequence
  Synthetic: NP3_B3
EVQLQQSGTVLARPGASVKMSCKASGSSFTTYWMHWVKQRPGQGLEWIGAIYPGNSDTSYNQKFKGKAKLTAVTSASTAYMELSSLTNEDSAVYYCTRKELFGYGFDYWGQGTTLTVSS

SEQ ID NO: 12 - protein, 121 aa, Artificial Sequence
  Synthetic: NP3_B4
EVQLVESGGGLVQPGGSVKLSCLASGFTFSDYYMSWVRQSPEKGLEWVAEIRLESDNYATHYAESVKGKFTISRDDSKSRLYLKMNSLRGEDTGIYYCAFDVYYGGAMDYWGQGTTVTVSS

SEQ ID NO: 13 - protein, 120 aa, Artificial Sequence
  Synthetic: NP3_B5
QVQLQQSGAELVRPGASVKLSCKASGYTFTSYWMNWVKQRPEQGLAWIGRIDPYDNATHYNHKFKDKAILTVDKSSSTAYMELRGLTSEDSAVYYCAIYYDYDDYIMDYWGQGTSVTVSS

SEQ ID NO: 14 - protein, 119 aa, Artificial Sequence
  Synthetic: NP3_B8
DVQLQESGGGLVKPGGSLKLSCAASGFTFSDYYMYWVRQTPERRLEWVATISDGGSYTYYPDSVKGRFTSSRDNAKNNLYLQMSSLKSEDTAMYYCTRGDDYGGRLAYWGQGTLVTVSA

SEQ ID NO: 15 - protein, 121 aa, Artificial Sequence
  Synthetic: NP3_B9
EVQLQQSGPELKKPGETVKISCKTSGYTFTNCGMHWVKQAPGKGLKWMGWINTYTGEPTYADDFKGRFAFSLETSVNTAYLQINNLKSEDTASYFCARRDDVYYVAGFDYWGQGTTLTVSS

SEQ ID NO: 16 - protein, 115 aa, Artificial Sequence
  Synthetic: NP3_B10
QIQLVQSGPELKKPGETVKISCKASGYTFTDYSMHWVKQAPGKDLKWVGWINTETGEPTYADDFKGRFAFSLETSATTAYLQINNLKNEDTATYFCARGAATRYWGQGTTLTVSS

SEQ ID NO: 17 - protein, 123 aa, Artificial Sequence
  Synthetic: NP3_B12
EVQLQQSGAELMKPGASVKISCKATGYTFSSYWIEWVKQRPGHGLEWIGEFFPGSGNTNYNEKFKGKATFTADTSSNTAYMQLSSLTSEDSAVYYCARRGLYDGYYVRYFDVWGAGTTVTVSS

SEQ ID NO: 18 - protein, 120 aa, Artificial Sequence
  Synthetic: NP3_C3
QIQLVQSGPELRKPGETVKISCKASGYIFTNYGMNWVKQAPGKGLKWMGWINTYTGEPTYAGDFKGRFAFSLETSASTAYLQINNLKNEDSATYFCARSHDNYDNHFDYWGQGTTLTVSS

SEQ ID NO: 19 - protein, 113 aa, Artificial Sequence
  Synthetic: NP3_C4
QIQLVQSGPELVKPGASVKISCKASGYSFTNYYMHWVRQSHVKSLEWIGRINPYSGATSYNQHFKDKASLTVDKSSSTAYMELHSLTSEDSAVYYCADNGYLWGQGTSVTVSS

SEQ ID NO: 20 - protein, 121 aa, Artificial Sequence
  Synthetic: NP3_C5
QIQLVQSGPELKTPGETVKISCKASGYTFTNCGMHWVKQAPGKGLKWMGWINTYTGEPIYADDFKRRFAFSLETSASTAYLQINNLNNEDTASYFCARRDDVYYVAGFDYWGQGTTVTVFS

SEQ ID NO: 21 - protein, 123 aa, Artificial Sequence
  Synthetic: NP3_C6
QVQLQQSGPELVKPGASVKMSCKASGYTFTDYVINWVKERTGQGLEWIGEIYPGSGDTYYNEKFKGKATLTADKSSNTAYMHLSSLTSEDSAVFFCARRKFYDGNYVRYFDVWGAGTTLTVSS

SEQ ID NO: 22 - protein, 118 aa, Artificial Sequence
  Synthetic: NP3_C8
EVQLQQSGAELARPGASVKMSCKASGYTFTSYTMHWVKQRPGQGLEWLGYFNPSSGYTNYVQRFRDKATLTADKSSSTAYMQLSSLTSEDSAVYYCARAGEGYAMDYWGQGTSVTVSS

SEQ ID NO: 23 - protein, 115 aa, Artificial Sequence
  Synthetic: NP3_C9
QIQLVQSGPELKKPGETVKISCKASGYTFTDYSIHWVKQAPGKDLKWVGWINTETGEPTYADDFEGRFAFSLETSASTAFLQINNLKNEDTATYFCSRGAATGFWGQGTTLTVSS

SEQ ID NO: 24 - protein, 119 aa, Artificial Sequence
  Synthetic: NP3_C10
QVTLKESGPGILKPSQTLSLTCSFSGFSLSTSGLGVGWIRQPSGKGLEWLAHIWWDDDKYYNPSLKSQLTISKETSRNQVFLKISSVDSADTATYYCARTVYHGAMDYWGQGTSVTVSS

SEQ ID NO: 25 - protein, 121 aa, Artificial Sequence
  Synthetic: NP3_C11
EVQLVESGGGLVRPGGSLKLSCAASGFTFSDYYMYWVRQTPEKRLEWVATISDGGGYNYYPDSVKGRFTVSRDNAKNNLYLQMSSLKSEDTAMYYCVRDKYDGWGSDFDVWGAGTTVTVSS

SEQ ID NO: 26 - protein, 113 aa, Artificial Sequence
  Synthetic: NP3_D1
DVQLQESGPELAKPGASVKVSCKASGYTFTSYNIYWVKQSHGKSLEWIGYIDPYNGGTSYNQKFKGKATLTVDKSSSTAYMHLNSLTSEDSAVYYCALPWDYWGQGTSVTVSS

SEQ ID NO: 27 - protein, 118 aa, Artificial Sequence
  Synthetic: NP3_D4
EVQLVESGPSLVKPSQTLSLTCSVTGDSITSGYWNWIRKFPGNKFEFMGYMRYSGSTYFSPSLKSRISITRDTSKNHYYLQLSSVTTEDTATYYCARGLRFFYAMDYWGQGTSVTVSS

SEQ ID NO: 28 - protein, 116 aa, Artificial Sequence
  Synthetic: NP3_D5
DVQLQESGPELVRPGVSVKISCKGSGYTFTDYVMHWVKQSHAKSLEWIGVISTYSGNTNYNQKFKGKATMTVDKFSNTAYMELARLTSEDSAIYYCTRGTGDFDYWGQGTTLTVSS

SEQ ID NO: 29 - protein, 117 aa, Artificial Sequence
  Synthetic: NP3_E2
EVQLQQSGPEVVRPGVSVKISCKGSGYTFTDYALHWVKQSHTKSLEWIGVISTYSGTTNYNQNFKGKATMAVDKSSSTAYMELARLTSEDSAIYYCASGGNYGLDYWGQGTSVTVSS

SEQ ID NO: 30 - protein, 120 aa, Artificial Sequence
  Synthetic: NP3_E3
QIQLVQSGPDLKKPGETVKISCKASGYTFTDYSMHWVKQVPGKGLKWMGWINTETDKPTYADDFKGRFAFSLETSASTAYSQINNLKNEDTATYFCARHYKYDEAWSPYWGQGTLVTVSA

SEQ ID NO: 31 - protein, 119 aa, Artificial Sequence
  Synthetic: NP3_E4
EVQLVESGPGLVAPSQSLSITCTVSGFSLTSYGVSWVRQPPGKGLEWLGVIWGDGSTNYHSALISRLSINKDNSKSQVFLRLNSLQTDDTATYYCAKEDDGYLFYFDYWGQGTTLTVSS

SEQ ID NO: 32 - protein, 119 aa, Artificial Sequence
  Synthetic: NP3_E5
EVQLVESGGGLVKPGGSLKLSCAASGFTFSDYYMSWVRQTPEKRLEWVATISDGGNYTYYPDSVKGRFTISRDNAKNNLYLQMSSLKSEDTAMYYCARGSTYGNPLDHWGQGTTLTVSS

SEQ ID NO: 33 - protein, 119 aa, Artificial Sequence
  Synthetic: NP3_E6
EVQLQQSGAELMRPGASVKISCKATGYTFSSYWIEWVKQRPGHGLEWIGEILPGSDNTNYNEKFKGKATLTADTSSNTAYMHLSSLTSEDSAVYSCARRLFLRRYFDVWGAGTTVTVSS

SEQ ID NO: 34 - protein, 116 aa, Artificial Sequence
  Synthetic: NP3_E11
EVQLQQSGPELVRPGVSVKISCKGSGYTFTDYVMHWVKQSHAKSLEWIGVISTYSGNTNYNQKFKGKATMTVDKSSNTVYMELARLTSEDSAIYYCTRGTGDFDYWGQGTTLTVSS

SEQ ID NO: 35 - protein, 122 aa, Artificial Sequence
  Synthetic: NP3_F4
EVQLVESGPELKKPGETVKIPCKTSGYTFTNYGMNWVKQAPGKGLKWMGWINTNTGEPTYAEDFKGRFAFSLETSASTAYLQISNLKNEDTATFFCARGSIYDGYYEGLYYWGQGTTLTVSS

SEQ ID NO: 36 - protein, 118 aa, Artificial Sequence
  Synthetic: NP3_F7
EVQLQQSGAELVRPGSSVKISCKASGYAFSSYWMNWVKQRPGQGLEWIGQIYPGNGDTYYNGKFMGKATLTADKFSSTAYMQLSGLTSEDSAVYFCARRPLFYAMDYWGQGTSVTVSS

SEQ ID NO: 37 - protein, 122 aa, Artificial Sequence
  Synthetic: NP3_F8
QVQLQQSGHGLVAPSQSLSITCTVSGFSLSSYGVHWVRQPPGKGLEWLAVIWSDGNTMYNSVLKSRLNISRDYSKSQVFLKGNSLQTDDTAMYYCARNSGYNDYDGYYFDVWGAGTSVTVSS

SEQ ID NO: 38 - protein, 124 aa, Artificial Sequence
  Synthetic: NP3_F10
EVQLQQSGAELVEPGAPVKLSCKAFGYTFTSYWMNWVKQRPGRGLEWIGRIDPSDSETHYNQNFKDKAALTVDRSSSTAYIHLSSLTSEDSAVYYCAREVTTATKGVFSWLAYWGQGTLVTVSA

SEQ ID NO: 39 - protein, 122 aa, Artificial Sequence
  Synthetic: NP3_G1
EVQLVESGAELVKPGASVKLSCKASGYTFTSYYMYWVQLRPGQGLEWIGEINPSNGGTNFNEKFKSKATLTVDKSSSTAYMQLSSLTSEDSAVYYCTRLEGYGDYDYAMYYWGQGTSVTVSS

SEQ ID NO: 40 - protein, 119 aa, Artificial Sequence
  Synthetic: NP3_G5
EVQLVESGPELKKPGEPVRISCKASGYTFTDYGMNWVKQAPGKGLKWMGWINTYTGEPTYADDFKGRFAFSLETSASTAFLQITNLKNEDTATYFCARAPLLRSYFDYWGQGTSVTVSS

SEQ ID NO: 41 - protein, 119 aa, Artificial Sequence
  Synthetic: NP3_G6
EVQLVESGGGLVKPGGSLKLSCAASGFTFSDYYMSWVRQTPEKRLEWVATISDGGSYTYYPDSVKGRFTISRDNAKNNLYLHMSSLKSEDTAMYYCARGSTYGNPLDYWGQGTSVTVSS

SEQ ID NO: 42 - protein, 118 aa, Artificial Sequence
  Synthetic: NP3_G7
QIQLVQSGAELVRPGSSVKISCKASGYAFSSYWMNWVKQRPGQGLEWIGQIYPGNGDTYYNGKFMGKATLTADKFSSTAYMQLSSLTSEDSTVYFCARRPLFYAMDYWGQGTSVTVSS

SEQ ID NO: 43 - protein, 118 aa, Artificial Sequence
  Synthetic: NP3_G8
QVQLQQSGPELKKPGETVKISCKASGYTFTDYSMHWVKQAPGKGLKWLGWKNTESGEETFADDFKGRFDFSLETSASTVYLQIKNLKNEDTAAYFCARSGDIYAMDYWGQGTTVTVSS

SEQ ID NO: 44 - protein, 118 aa, Artificial Sequence
  Synthetic: NP3_G9
EVQLQQSGAELVRPGSSVKISCKASGYAFSNYWMNWVRQRPGQGLEWIGQIYPGNDDTYYNGKFKGKVTLTADKSSTTAYIQLISLTSEDSAVYFCARRPLFYAMDYWGQGTSVTVSS

SEQ ID NO: 45 - protein, 119 aa, Artificial Sequence
  Synthetic: NP3_G12
EAQLQQSGAEPMKPGASVKISCKTTGYTFTSDWIEWVQQRPGHGLEWIGEILPGSGNTNYNENFKGKATLTADTSSNTAYMHLSSLTSEDSAVYYCARRLFLRRYFDVWGAGTTVTVSS

SEQ ID NO: 46 - protein, 118 aa, Artificial Sequence
  Synthetic: NP3_H2
EVQLQQSGAELARPGASVKMSCKASGYIFTSYTIHWIKQRPGQGLEWIGYFNPSSDYFSYIQIFKDKATLTADKFSSTAYMQLSSLTSEDSAVYYCARAGEVYAKDYWGQGTSVTVSS

SEQ ID NO: 47 - protein, 120 aa, Artificial Sequence
  Synthetic: NP3_H9
QIQLVQSGPELKKPGETVKISCKASGYTFTDYPIHWVKQAPGKGLKWMGWINAETGEPTYADDFKGRFVVSLETSASTAYLQINNLKNEDTATFFCARHYRYDEAWFVYWGQGTLVTVSA

SEQ ID NO: 48 - protein, 121 aa, Artificial Sequence
  Synthetic: NP3_H12
QIQLVQSGPELKKPGETVKISCKASGYMFTNYGMNWVKQAPGKGLKWMGWINTYTGEPTYADDFKGRFAFSLDISGSTAYLQINNLKNEDMATYFCARTREIWGNVDFPYWGQGTLVTVSA

SEQ ID NO: 49 - protein, 107 aa, Artificial Sequence
  Synthetic: NP3_A3
DIVMTQSPASLSVSVGETVTITCRSSENIYSNLAWFQQKQGKSPQLLVYGATDLADGVPSRFSGSGSGTQYSLKINSLQSEDFGTYYCQHFWGTPWTFGGGTKLEIK

SEQ ID NO: 50 - protein, 107 aa, Artificial Sequence
  Synthetic: NP3_A4
DIVMTQSHKFMSTSVGDRVSITCKASQDVSTAVAWYQQKPGQSPKLLIYSASHRYTGVPDRFTGSGSGTDFTFTISSVQAEDLAVYYCQQHYSNPLTFGAGTKLEIK

SEQ ID NO: 51 - protein, 107 aa, Artificial Sequence
  Synthetic: NP3_A6
DIQMNQSPSSLSVSVGETVTITCRTSENIYSNLAWYQQKQGKSPQLLVYGATDLADGVPSRFSGSGSGTQYSLKISSLQSEDFGTYYCQHFWGTPWTFGGGTKLEIK

SEQ ID NO: 52 - protein, 112 aa, Artificial Sequence
  Synthetic: NP3_A7
DIQMTQSPASVPVTPGESVSISCRSSKSLLHSNGNTYVYWFLQRPGQSPRLLIYRMSNLASGVPDRFSGSGSGTAITLRISRVEAEDVGVYSCMQHLEYPLTFGAGTKLEIK

SEQ ID NO: 53 - protein, 111 aa, Artificial Sequence
  Synthetic: NP3_A8
DIVMTQSPASLVVSLGQRATISCRASKSVSASGYSYIHWYQQIPGQPPKLLIYLASNLESGVPARFSGSGSGTDFTLNIHPVEEEDAATYYCQHSRELPLTFGTGTKLEIK

SEQ ID NO: 54 - protein, 112 aa, Artificial Sequence
  Synthetic: NP3_A9
ENVLTQSPASNPVTLGTSASISCRSSKSLLHSNGFTYLYWYLQKPGQSPQLLIYQMSYLASGVPDRFSSSGSGTDFTLRISRVEAEDVGVYYCAQNLELPFTFGSGTKLEIK

SEQ ID NO: 55 - protein, 111 aa, Artificial Sequence
  Synthetic: NP3_A10
ENVLTQSPASLTVSLGQRATLSCKASQSVDYDGDSYMNWYQQKPGQPPKLLIYAASYLQSGIPARFSGSGSGTDFTLNIHPVEEEDAATYYCQQSIEDPYTFGGGTKLEIK

SEQ ID NO: 56 - protein, 107 aa, Artificial Sequence
  Synthetic: NP3_A11
DIQMTQSPSSLSVSVGETVTITCRASENIYSSLAWYQQKQGKSPQLLVYTATDLADGVPSRFSGSGSGSQYSLRINSLQSEDFGSYYCQHFWGFPWTFGGGTKLEIK

SEQ ID NO: 57 - protein, 112 aa, Artificial Sequence
  Synthetic: NP3_B1
DVVMTQTPLTLSVTIGQPASISCKSSQTLLDSDGKTYLNWLFQRPGQSPKRLIYLVSELDSGVPDRFTGSGSGTDFTLKISRVEAEDLGVYYCWQGTHFPQTFGGGTKLELK

SEQ ID NO: 58 - protein, 107 aa, Artificial Sequence
  Synthetic: NP3_B2
DIQMTQTTSSLSASLGDRVTINCSASQGISNYLNWYQQKPDGTVKLLIYFTSSLHSGFPSRFSGSGSGTDFSLTISNLEPEDIATYYCQQYSKLPFTFGSGTKLEIK

SEQ ID NO: 59 - protein, 111 aa, Artificial Sequence
  Synthetic: NP3_B3
DIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLELK

SEQ ID NO: 60 - protein, 106 aa, Artificial Sequence
  Synthetic: NP3_B4
DIQMTQSPSIMSASPGEKVTMTCSASSSVSYMHWYQQKSSTSPKLWIYDTSELASGVPGRFSGSRSGNSYSLTISSMEAEDVATYYCFQGSGYPLTFGGGTKLELK

SEQ ID NO: 61 - protein, 106 aa, Artificial Sequence
  Synthetic: NP3_B5
ENVLTQSPAIMSASPGEKVTMTCSASSSVNYMHWYQQRPGSSPKPWIYDTSNLASGIPVRFSGSGSGTSYSLIISSMEAEDAATYYCHQRSSFPFTFGSGTKLEIK

SEQ ID NO: 62 - protein, 107 aa, Artificial Sequence
  Synthetic: NP3_B8
DIQMNQSPSSLSASLGGRVTISCSASQGISNYLNWYQQKPDGTVKLLIYYTSSLHSGVPSRFSGSGSGTDYSLTISNLEPEDIATYYCQQYNKLPYTFGGGTKLEIK

SEQ ID NO: 63 - protein, 107 aa, Artificial Sequence
  Synthetic: NP3_B9
DIQMTQSPASLSVSVGETVTITCRASENIYSNLAWYQQKQGKSPQLLVNGATDLADGVPSRFSGSGSGTQYSLKINSLQSEDFGSYYCQLFWGTPWTFGGGTKLEIK

SEQ ID NO: 64 - protein, 111 aa, Artificial Sequence
  Synthetic: NP3_B10
DIVMTQSPSSLAVSLGQRATISCKASQSVDFDGKSYMNWYQQKPGQPPKLLIYVASNLESGIPARFSGSGSGTDFTLNIHPVEEEDAATYYCQQTNEDPLTFGTGTKLEIK

SEQ ID NO: 65 - protein, 111 aa, Artificial Sequence
  Synthetic: NP3_B12
DIVLTQSPASLAVSLGQRATISCKASQSVDYDGYSYMNWYQQKPGQPPKLLIYAASNLESGIPARFSGSGSGTDFTLNIHPVEEEDAATYYCQQSNADPFTFGGGTKLELK

SEQ ID NO: 66 - protein, 107 aa, Artificial Sequence
  Synthetic: NP3_C3
DIVMTQSPASLSVSVGETVTITCRASENIYSNLAWYQQKQGKSPQLLVYVATDLADGVPSRFSGSGSGTQYSLKINSLQSEDFGSYYCQHFWGTPWTFGGGTKLELK

SEQ ID NO: 67 - protein, 112 aa, Artificial Sequence
  Synthetic: NP3_C4
DIVMTQAAPTLSVTIGQPASISCKSSQSLLDSDGKTYLNWLLQRPGQSPRRLIYLVSKLDSGVPDRFTGSGSGTDFTLKISRVEAEDLGVYYCWQGTHFPRTFGGGTKLEIK

SEQ ID NO: 68 - protein, 107 aa, Artificial Sequence
  Synthetic: NP3_C5
DIQMTQSPASLSVSVGETVTITCRASENIYSNLAWYQQKQGKSPQLLVYGATDLADGVPSRFSGSGSGTQYSLKINSLQSEDFGSYYCQHFWGTPWTFGGGTKLELK

SEQ ID NO: 69 - protein, 111 aa, Artificial Sequence
  Synthetic: NP3_C6
DIKMTQSPSSLAVSLGQRATISCKASQSVDYDGDSYMNWYQQRPGQPPKLLIYAASNLESGIPARFSGSGSGADFTLNIHPVEEEDAATYYCQQSNEDPWTFGGGTKLEIK

SEQ ID NO: 70 - protein, 111 aa, Artificial Sequence
  Synthetic: NP3_C8
DIVMTQSPASLAVSLGQRATISCKASQSVDYDGDSYMSWYLQKPGQPPKLLIYAASNLESGIPARFSGSGSGTDFTLNIHPVEEEDAATYYCQQSIGDPWTFGGGTKLEIK

SEQ ID NO: 71 - protein, 111 aa, Artificial Sequence
  Synthetic: NP3_C9
DIVLTQSPASLAVSLGQRATISCKASQSVDYDGDSYMNWYQQKPGQPPKLLIYVASNLESGIPARFSGSGSGTDFTLNIHPVEDEDAATYYCQQTNEDPLTFGAGTKLELK

SEQ ID NO: 72 - protein, 106 aa, Artificial Sequence
  Synthetic: NP3_C10
DIKMTQSPSIMSASPGEKVTMTCSASSSVSYMYWFQQKPGSSPRLLIYDTSNLASGVPVRFSGSGSGTSYSLTISRMEAEDAATYYCQQWSGYPYTFGGGTKLEIK

SEQ ID NO: 73 - protein, 108 aa, Artificial Sequence
  Synthetic: NP3_C11
DIVMTQSPSLMSTSLGERVTMTCTANSSVSSNYMHWYQQKPGSSPKLWIYSTSNLASGVPARFSGSGSGTSYSLTISNMEAEDAATYYCHQFHRSPRTFGGGTKLEIK

SEQ ID NO: 74 - protein, 112 aa, Artificial Sequence
  Synthetic: NP3_D1
DIVMTQAAPTLSVTLGQPASISCKSSQSLLDSDGKTYLSWLLQRPGQSPKRLIYLVSELDSGVPDRFTGSGSGTDFTLKISRVEAEDLGVYYCWQGTHFPQTFGGGTKLEIK

SEQ ID NO: 75 - protein, 111 aa, Artificial Sequence
  Synthetic: NP3_D4
ENVLTQSPASLTVSLGQRATLSCKASQSVDYDGDSYMNWYQQKPGQPPKLLIYAASYPESGIPARFSGSGSGTDFTLNIHPVEEEDAATYYCQQSIEDPYTFGGGTKLEIK

SEQ ID NO: 76 - protein, 107 aa, Artificial Sequence
  Synthetic: NP3_D5
DIVMTQSHKFMSTSVGDRVSITCKASQDVGTAVAWYQQKPGQSPKLLIYWASTRHTGVPDRFTGSGSGTDFTLTISNVQSEDLADYFCHQYSSYPYTFGGGTKLEIK

SEQ ID NO: 77 - protein, 107 aa, Artificial Sequence
  Synthetic: NP3_E2
DIVMTQSHKFMSTSVGDRVSITCKANQDVGSAVAWYQEKPGQSPKLLIYWTSTRHTGVPDRFTGNGSGTDFTLTISNVQSEDLVDYFCQQYSRYPLTFGSGTKLELK

SEQ ID NO: 78 - protein, 107 aa, Artificial Sequence
  Synthetic: NP3_E3
DIVLTQSPASLSVSVGETVTITCRASENIYSNLAWYQQKQGQSPQLLVYAATDLADGVPSRFSGSGSGTQYSLKINSLQSEDFGNYYCQHFWGTPWTFGGGTKLEIK

SEQ ID NO: 79 - protein, 107 aa, Artificial Sequence
  Synthetic: NP3_E4
DIKMTQSPSSLSASLGDRVTISCSASQDISDYLNWYQQKPDGTVKLLIYYTSSLHSGVPSRFSGSGSGTDYSLTISNLEPEDIATYYCQQYSKLPYTFGGGTKLEIK

SEQ ID NO: 80 - protein, 107 aa, Artificial Sequence
  Synthetic: NP3_E5
DIQMTQTTSSLSASLGDRVTINCSASQGISNYLNWYQQKPDGTVKLLIYYTSILHSGVPSRFSGSGSGTDYSLTISNLEPEDIATYYCQQYSKLPFTFGSGTKLEIK

SEQ ID NO: 81 - protein, 111 aa, Artificial Sequence
  Synthetic: NP3_E6
DIVLTQSPASLALSLGQRATISCKASQSVDYDGDSYMNWYQQKPGQPPKLLISAASNLESGIPARFGGSGSGTDFTLNIHPVEEEDAATYYCQQSNEDPFTFGGGTKLEIK

SEQ ID NO: 82 - protein, 107 aa, Artificial Sequence
  Synthetic: NP3_E11
DIVMTQSHKFMSTSVGDRVSITCKASQDVGSAVAWYQQKPGQSPKLLIYWTSTRHTGVPDHFTGSGSGTDFTLTINNVQSEDLADYFCHQYSSYPYTFGGGTKLEIK

SEQ ID NO: 83 - protein, 107 aa, Artificial Sequence
  Synthetic: NP3_F4
DIKMTQSPSSLSVSVGETVTITCRASENIYSNLAWYQQKQGKSPQLLVSAATNLADNVPSRFSGSGSGTQYSLKINSLQSEDFGSYYCQHFWGFPRTFGGGTKLEIK

SEQ ID NO: 84 - protein, 111 aa, Artificial Sequence
  Synthetic: NP3_F7
DIVLTQSPGSLAVSLGQRATISCKASQSVDYDGDSYMNWYQQKPGQPPKLLIYAASNLESGIPVRFSGSGSGTDFTLNIHPVEEEDAATYYCQQSNEDPWTFGGGTKLELK

SEQ ID NO: 85 - protein, 108 aa, Artificial Sequence
  Synthetic: NP3_F8
DIQMTQSPSIMAASVGQKVTMTCSASSSVSSSYLHWYQQKSGASPKPLIHRTSNLASGVPPRFSGSGSGTSYSLTISSVEAEDDATYYCQQWSGYPFTFGTGTKLEIK

SEQ ID NO: 86 - protein, 107 aa, Artificial Sequence
  Synthetic: NP3_F10
DIQMTQTTSSLSASLGDRVTISCSASQDISDYLNWYQQKPDGTIKLLIYYTSSLHSGAPSRFSGSGSGTDYSLTISNLEPEDIATYYCQQYSKLPWTFGGGTKLELK

SEQ ID NO: 87 - protein, 111 aa, Artificial Sequence
  Synthetic: NP3_G1
DIVLTQSPASLAVSLGQRATISCRASKSVSASGYSYLHWYQQKPGQPPKLLIYLASNLPSGVPARFSGSGSGTDFTLNIHPVEEEDAATYYCQHRREIPLTFGAGTKLEIK

SEQ ID NO: 88 - protein, 107 aa, Artificial Sequence
  Synthetic: NP3_G5
DIVMTQSHKFMSTSVGDRISITCKASQDVSTAVVWYQQKPGQSLKLLIYSASYRYTGVPDRFTGSGSGTDFTFTISSVQAEDLAIYYCQQYYSTPFTFGSGTKLEIK

SEQ ID NO: 89 - protein, 107 aa, Artificial Sequence
  Synthetic: NP3_G6
DIQMTQSPASLSASLGDRVTINCSASQGISNYLNWYQQKPDGTVKLLIYFTSSLHSGFPSRFSGSGSGTDFSLTISNLEPEDIATYYCQQYSKLPFTFGSGTKLEIK

SEQ ID NO: 90 - protein, 107 aa, Artificial Sequence
  Synthetic: NP3_G7
DIQMTQSPASLSVSVGETVTITCRASENIYSNLAWYQQKQGKSPQLLVYVATDLADGVPSRFSGSGSDTQYSLKINSLQSEDFGSYYCQHFWGTPWTFGGGTKLEIK

SEQ ID NO: 91 - protein, 111 aa, Artificial Sequence
  Synthetic: NP3_G8
ENVLTQSPASLAVSLGQRATISCKASQSVDYDGGSYMNWYQQKPGQPPKLLIYAASTLESGIPAGFSGSGSGTDFTLNIHPVEEEDAATYYCQQSNEDPWTFGGGTKLEIK

SEQ ID NO: 92 - protein, 111 aa, Artificial Sequence
  Synthetic: NP3_G9
DIQMTQSPSSLAVSLGQRASISCKASQSVDYDGESYMNWYQQKPGQPPKVLIYAASNLESGIPARFSGSGAGTDFTLSIHPVEEEDAATYYCQQSYEDPWTFGGGTKLELK

SEQ ID NO: 93 - protein, 111 aa, Artificial Sequence
  Synthetic: NP3_G12
DIVMTQSPASLAVSLGQRATISCKASQSVDYDGDSYMNWYQQKPGQPPKLLIYAASNLESGIPARFGGGGSGTDFTLNIHPVEEEDAATYYCQQSNEDPFTFGGGTKLELK

SEQ ID NO: 94 - protein, 111 aa, Artificial Sequence
  Synthetic: NP3_H2
ENVLTQSPASLAVSLGQRATISCKASQSVDYDGDSYMNWYQQKPGQPPKLLIYAASSLDSGIPARFGGSGSGTDFTLNIHPVEEEDAATYYCQQSNEDPWTFGGGTKLEIK

SEQ ID NO: 95 - protein, 107 aa, Artificial Sequence
  Synthetic: NP3_H9
DVQITQSPSSLSVSVGETVTITCRASENIYSNLAWYQQKQGKSPQLLVYAATDLADGVPSRFSGSGSGTHYSLKINSLQSEDFGSFYCQHFWGTPWTFGGGTKLEIK

SEQ ID NO: 96 - protein, 107 aa, Artificial Sequence
  Synthetic: NP3_H12
DILMTQAAPSLSVSVGETVTITCRASENIYSDLAWYQHKEGKSPQLLVYAATDLADGVPSRFSGSGSGTQYSLKINSLQSEDFGNYYCQHFWGTPWTFGGGTKLEIK

SEQ ID NO: 97 - protein, 8 aa, Artificial Sequence
  Synthetic: NP3_A3
GYTFTDYA

SEQ ID NO: 98 - protein, 10 aa, Artificial Sequence
  Synthetic: NP3_A4
GFSLSTSGMG

SEQ ID NO: 99 - protein, 8 aa, Artificial Sequence
  Synthetic: NP3_A6
GYTFTDYS

SEQ ID NO: 100 - protein, 8 aa, Artificial Sequence
  Synthetic: NP3_A7
GFNIKDTY

SEQ ID NO: 101 - protein, 8 aa, Artificial Sequence
  Synthetic: NP3_A8
GYTFTSYY

SEQ ID NO: 102 - protein, 8 aa, Artificial Sequence
  Synthetic: NP3_A9
GYTFTDYN

SEQ ID NO: 103 - protein, 8 aa, Artificial Sequence
  Synthetic: NP3_A10
GDSVTSGY

SEQ ID NO: 104 - protein, 7 aa, Artificial Sequence
  Synthetic: NP3_A11
GYTFTNY

SEQ ID NO: 105 - protein, 8 aa, Artificial Sequence
  Synthetic: NP3_B1
GYTFTTYN

SEQ ID NO: 106 - protein, 8 aa, Artificial Sequence
  Synthetic: NP3_B2
GFTFSDYY

SEQ ID NO: 107 - protein, 8 aa, Artificial Sequence
  Synthetic: NP3_B3
GSSFTTYW

SEQ ID NO: 108 - protein, 8 aa, Artificial Sequence
  Synthetic: NP3_B4
GFTFSDYY

SEQ ID NO: 109 - protein, 8 aa, Artificial Sequence
  Synthetic: NP3_B5
GYTFTSYW

SEQ ID NO: 110 - protein, 8 aa, Artificial Sequence
  Synthetic: NP3_B8
GFTFSDYY

SEQ ID NO: 111 - protein, 8 aa, Artificial Sequence
  Synthetic: NP3_B9
GYTFTNCG

SEQ ID NO: 112 - protein, 8 aa, Artificial Sequence
  Synthetic: NP3_B10
GYTFTDYS

SEQ ID NO: 113 - protein, 8 aa, Artificial Sequence
  Synthetic: NP3_B12
GYTFSSYW

SEQ ID NO: 114 - protein, 8 aa, Artificial Sequence
  Synthetic: NP3_C3
GYIFTNYG

SEQ ID NO: 115 - protein, 8 aa, Artificial Sequence
  Synthetic: NP3_C4
GYSFTNYY

SEQ ID NO: 116 - protein, 8 aa, Artificial Sequence
  Synthetic: NP3_C5
GYTFTNCG

SEQ ID NO: 117 - protein, 8 aa, Artificial Sequence
  Synthetic: NP3_C6
GYTFTDYV

SEQ ID NO: 118 - protein, 8 aa, Artificial Sequence
  Synthetic: NP3_C8
GYTFTSYT

SEQ ID NO: 119 - protein, 8 aa, Artificial Sequence
  Synthetic: NP3_C9
GYTFTDYS

SEQ ID NO: 120 - protein, 10 aa, Artificial Sequence
  Synthetic: NP3_C10
GFSLSTSGLG

SEQ ID NO: 121 - protein, 8 aa, Artificial Sequence
  Synthetic: NP3_C11
GFTFSDYY

SEQ ID NO: 122 - protein, 7 aa, Artificial Sequence
  Synthetic: NP3_D1
GYTFTSY

SEQ ID NO: 123 - protein, 8 aa, Artificial Sequence
  Synthetic: NP3_D4
GDSITSGY

SEQ ID NO: 124 - protein, 8 aa, Artificial Sequence
  Synthetic: NP3_D5
GYTFTDYV

SEQ ID NO: 125 - protein, 8 aa, Artificial Sequence
  Synthetic: NP3_E2
GYTFTDYA

SEQ ID NO: 126 - protein, 8 aa, Artificial Sequence
  Synthetic: NP3_E3
GYTFTDYS

SEQ ID NO: 127 - protein, 8 aa, Artificial Sequence
  Synthetic: NP3_E4
GFSLTSYG

SEQ ID NO: 128 - protein, 8 aa, Artificial Sequence
  Synthetic: NP3_E5
GFTFSDYY

SEQ ID NO: 129 - protein, 8 aa, Artificial Sequence
  Synthetic: NP3_E6
GYTFSSYW

SEQ ID NO: 130 - protein, 8 aa, Artificial Sequence
  Synthetic: NP3_E11
GYTFTDYV

SEQ ID NO: 131 - protein, 8 aa, Artificial Sequence
  Synthetic: NP3_F4
GYTFTNYG

SEQ ID NO: 132 - protein, 8 aa, Artificial Sequence
  Synthetic: NP3_F7
GYAFSSYW

SEQ ID NO: 133 - protein, 8 aa, Artificial Sequence
  Synthetic: NP3_F8
GFSLSSYG

SEQ ID NO: 134 - protein, 8 aa, Artificial Sequence
  Synthetic: NP3_F10
GYTFTSYW

SEQ ID NO: 135 - protein, 8 aa, Artificial Sequence
  Synthetic: NP3_G1
GYTFTSYY

SEQ ID NO: 136 - protein, 8 aa, Artificial Sequence
  Synthetic: NP3_G5
GYTFTDYG

SEQ ID NO: 137 - protein, 8 aa, Artificial Sequence
  Synthetic: NP3_G6
GFTFSDYY

SEQ ID NO: 138 - protein, 8 aa, Artificial Sequence
  Synthetic: NP3_G7
GYAFSSYW

SEQ ID NO: 139 - protein, 8 aa, Artificial Sequence
  Synthetic: NP3_G8
GYTFTDYS

SEQ ID NO: 140 - protein, 8 aa, Artificial Sequence
  Synthetic: NP3_G9
GYAFSNYW

SEQ ID NO: 141 - protein, 8 aa, Artificial Sequence
  Synthetic: NP3_G12
GYTFTSDW

SEQ ID NO: 142 - protein, 8 aa, Artificial Sequence
  Synthetic: NP3_H2
GYIFTSYT

SEQ ID NO: 143 - protein, 7 aa, Artificial Sequence
  Synthetic: NP3_H9
GYTFTDY

SEQ ID NO: 144 - protein, 8 aa, Artificial Sequence
  Synthetic: NP3_H12
GYMFTNYG

SEQ ID NO: 145 - protein, 8 aa, Artificial Sequence
  Synthetic: NP3_A3
INTETGEP

SEQ ID NO: 146 - protein, 6 aa, Artificial Sequence
  Synthetic: NP3_A4
IWWDDD

SEQ ID NO: 147 - protein, 8 aa, Artificial Sequence
  Synthetic: NP3_A6
INTETGEP

SEQ ID NO: 148 - protein, 8 aa, Artificial Sequence
  Synthetic: NP3_A7
IDPANGDT

SEQ ID NO: 149 - protein, 8 aa, Artificial Sequence
  Synthetic: NP3_A8
INPSNGGF

SEQ ID NO: 150 - protein, 8 aa, Artificial Sequence
  Synthetic: NP3_A9
INPDYGST

SEQ ID NO: 151 - protein, 7 aa, Artificial Sequence
  Synthetic: NP3_A10
ISYSGST

SEQ ID NO: 152 - protein, 8 aa, Artificial Sequence
  Synthetic: NP3_A11
INTYTGEP

SEQ ID NO: 153 - protein, 8 aa, Artificial Sequence
  Synthetic: NP3_B1
IDPYNGGT

SEQ ID NO: 154 - protein, 8 aa, Artificial Sequence
  Synthetic: NP3_B2
ISDGGSYT

SEQ ID NO: 155 - protein, 8 aa, Artificial Sequence
  Synthetic: NP3_B3
IYPGNSDT

SEQ ID NO: 156 - protein, 10 aa, Artificial Sequence
  Synthetic: NP3_B4
IRLESDNYAT

SEQ ID NO: 157 - protein, 8 aa, Artificial Sequence
  Synthetic: NP3_B5
IDPYDNAT

SEQ ID NO: 158 - protein, 8 aa, Artificial Sequence
  Synthetic: NP3_B8
ISDGGSYT

SEQ ID NO: 159 - protein, 8 aa, Artificial Sequence
  Synthetic: NP3_B9
INTYTGEP

SEQ ID NO: 160 - protein, 8 aa, Artificial Sequence
  Synthetic: NP3_B10
INTETGEP

SEQ ID NO: 161 - protein, 8 aa, Artificial Sequence
  Synthetic: NP3_B12
FFPGSGNT

SEQ ID NO: 162 - protein, 8 aa, Artificial Sequence
  Synthetic: NP3_C3
INTYTGEP

SEQ ID NO: 163 - protein, 8 aa, Artificial Sequence
  Synthetic: NP3_C4
INPYSGAT

SEQ ID NO: 164 - protein, 8 aa, Artificial Sequence
  Synthetic: NP3_C5
INTYTGEP

SEQ ID NO: 165 - protein, 8 aa, Artificial Sequence
  Synthetic: NP3_C6
IYPGSGDT

SEQ ID NO: 166 - protein, 8 aa, Artificial Sequence
  Synthetic: NP3_C8
FNPSSGYT

SEQ ID NO: 167 - protein, 8 aa, Artificial Sequence
  Synthetic: NP3_C9
INTETGEP

SEQ ID NO: 168 - protein, 7 aa, Artificial Sequence
  Synthetic: NP3_C10
IWWDDDK

SEQ ID NO: 169 - protein, 8 aa, Artificial Sequence
  Synthetic: NP3_C11
ISDGGGYN

SEQ ID NO: 170 - protein, 8 aa, Artificial Sequence
  Synthetic: NP3_D1
IDPYNGGT

SEQ ID NO: 171 - protein, 7 aa, Artificial Sequence
  Synthetic: NP3_D4
MRYSGST

SEQ ID NO: 172 - protein, 8 aa, Artificial Sequence
  Synthetic: NP3_D5
ISTYSGNT

SEQ ID NO: 173 - protein, 8 aa, Artificial Sequence
  Synthetic: NP3_E2
ISTYSGTT

SEQ ID NO: 174 - protein, 8 aa, Artificial Sequence
  Synthetic: NP3_E3
INTETDKP

SEQ ID NO: 175 - protein, 7 aa, Artificial Sequence
  Synthetic: NP3_E4
IWGDGST

SEQ ID NO: 176 - protein, 8 aa, Artificial Sequence
  Synthetic: NP3_E5
ISDGGNYT

SEQ ID NO: 177 - protein, 10 aa, Artificial Sequence
  Synthetic: NP3_E6
ILPGSDNTNY

SEQ ID NO: 178 - protein, 8 aa, Artificial Sequence
  Synthetic: NP3_E11
ISTYSGNT

SEQ ID NO: 179 - protein, 8 aa, Artificial Sequence
  Synthetic: NP3_F4
INTNTGEP

SEQ ID NO: 180 - protein, 8 aa, Artificial Sequence
  Synthetic: NP3_F7
IYPGNGDT

SEQ ID NO: 181 - protein, 7 aa, Artificial Sequence
  Synthetic: NP3_F8
IWSDGNT

SEQ ID NO: 182 - protein, 8 aa, Artificial Sequence
  Synthetic: NP3_F10
IDPSDSET

SEQ ID NO: 183 - protein, 8 aa, Artificial Sequence
  Synthetic: NP3_G1
INPSNGGT

SEQ ID NO: 184 - protein, 8 aa, Artificial Sequence
  Synthetic: NP3_G5
INTYTGEP

SEQ ID NO: 185 - protein, 8 aa, Artificial Sequence
  Synthetic: NP3_G6
ISDGGSYT

SEQ ID NO: 186 - protein, 8 aa, Artificial Sequence
  Synthetic: NP3_G7
IYPGNGDT

SEQ ID NO: 187 - protein, 8 aa, Artificial Sequence
  Synthetic: NP3_G8
KNTESGEE

SEQ ID NO: 188 - protein, 8 aa, Artificial Sequence
  Synthetic: NP3_G9
IYPGNDDT

SEQ ID NO: 189 - protein, 8 aa, Artificial Sequence
  Synthetic: NP3_G12
ILPGSGNT

SEQ ID NO: 190 - protein, 7 aa, Artificial Sequence
  Synthetic: NP3_H2
FNPSSDY

SEQ ID NO: 191 - protein, 8 aa, Artificial Sequence
  Synthetic: NP3_H9
INAETGEP

SEQ ID NO: 192 - protein, 8 aa, Artificial Sequence
  Synthetic: NP3_H12
INTYTGEP

SEQ ID NO: 193 - protein, 13 aa, Artificial Sequence
  Synthetic: NP3_A3
ARHYRYDEAWFAY

SEQ ID NO: 194 - protein, 11 aa, Artificial Sequence
  Synthetic: NP3_A4
ARSGYRGNFDY

SEQ ID NO: 195 - protein, 13 aa, Artificial Sequence
  Synthetic: NP3_A6
VRGYRYDEAWFVY

SEQ ID NO: 196 - protein, 10 aa, Artificial Sequence
  Synthetic: NP3_A7
ARYRIYYFDY

SEQ ID NO: 197 - protein, 15 aa, Artificial Sequence
  Synthetic: NP3_A8
TRLEGYGDSDYAMIY

SEQ ID NO: 198 - protein, 13 aa, Artificial Sequence
  Synthetic: NP3_A9
ARGDYDGGYGMDY

SEQ ID NO: 199 - protein, 12 aa, Artificial Sequence
  Synthetic: NP3_A10
ARGLRFFYAMDY

SEQ ID NO: 200 - protein, 13 aa, Artificial Sequence
  Synthetic: NP3_A11
ARSHGNYENHFDY

SEQ ID NO: 201 - protein, 6 aa, Artificial Sequence
  Synthetic: NP3_B1
ALPWDY

SEQ ID NO: 202 - protein, 12 aa, Artificial Sequence
  Synthetic: NP3_B2
ARGSTYGNPLDY

SEQ ID NO: 203 - protein, 12 aa, Artificial Sequence
  Synthetic: NP3_B3
TRKELFGYGFDY

SEQ ID NO: 204 - protein, 12 aa, Artificial Sequence
  Synthetic: NP3_B4
AFDVYYGGAMDY

SEQ ID NO: 205 - protein, 13 aa, Artificial Sequence
  Synthetic: NP3_B5
AIYYDYDDYIMDY

SEQ ID NO: 206 - protein, 12 aa, Artificial Sequence
  Synthetic: NP3_B8
TRGDDYGGRLAY

SEQ ID NO: 207 - protein, 14 aa, Artificial Sequence
  Synthetic: NP3_B9
ARRDDVYYVAGFDY

SEQ ID NO: 208 - protein, 8 aa, Artificial Sequence
  Synthetic: NP3_B10
ARGAATRY

SEQ ID NO: 209 - protein, 16 aa, Artificial Sequence
  Synthetic: NP3_B12
ARRGLYDGYYVRYFDV

SEQ ID NO: 210 - protein, 13 aa, Artificial Sequence
  Synthetic: NP3_C3
ARSHDNYDNHFDY

SEQ ID NO: 211 - protein, 6 aa, Artificial Sequence
  Synthetic: NP3_C4
ADNGYL

SEQ ID NO: 212 - protein, 14 aa, Artificial Sequence
  Synthetic: NP3_C5
ARRDDVYYVAGFDY

SEQ ID NO: 213 - protein, 16 aa, Artificial Sequence
  Synthetic: NP3_C6
ARRKFYDGNYVRYFDV

SEQ ID NO: 214 - protein, 11 aa, Artificial Sequence
  Synthetic: NP3_C8
ARAGEGYAMDY

SEQ ID NO: 215 - protein, 8 aa, Artificial Sequence
  Synthetic: NP3_C9
SRGAATGF

SEQ ID NO: 216 - protein, 11 aa, Artificial Sequence
  Synthetic: NP3_C10
ARTVYHGAMDY

SEQ ID NO: 217 - protein, 14 aa, Artificial Sequence
  Synthetic: NP3_C11
VRDKYDGWGSDFDV

SEQ ID NO: 218 - protein, 6 aa, Artificial Sequence
  Synthetic: NP3_D1
ALPWDY

SEQ ID NO: 219 - protein, 12 aa, Artificial Sequence
  Synthetic: NP3_D4
ARGLRFFYAMDY

SEQ ID NO: 220 - protein, 9 aa, Artificial Sequence
  Synthetic: NP3_D5
TRGTGDFDY

SEQ ID NO: 221 - protein, 10 aa, Artificial Sequence
  Synthetic: NP3_E2
ASGGNYGLDY

SEQ ID NO: 222 - protein, 13 aa, Artificial Sequence
  Synthetic: NP3_E3
ARHYKYDEAWSPY

SEQ ID NO: 223 - protein, 13 aa, Artificial Sequence
  Synthetic: NP3_E4
AKEDDGYLFYFDY

SEQ ID NO: 224 - protein, 12 aa, Artificial Sequence
  Synthetic: NP3_E5
ARGSTYGNPLDH

SEQ ID NO: 225 - protein, 12 aa, Artificial Sequence
  Synthetic: NP3_E6
ARRLFLRRYFDV

SEQ ID NO: 226 - protein, 9 aa, Artificial Sequence
  Synthetic: NP3_E11
TRGTGDFDY

SEQ ID NO: 227 - protein, 15 aa, Artificial Sequence
  Synthetic: NP3_F4
ARGSIYDGYYEGLYY

SEQ ID NO: 228 - protein, 11 aa, Artificial Sequence
  Synthetic: NP3_F7
ARRPLFYAMDY

SEQ ID NO: 229 - protein, 16 aa, Artificial Sequence
  Synthetic: NP3_F8
ARNSGYNDYDGYYFDV

SEQ ID NO: 230 - protein, 17 aa, Artificial Sequence
  Synthetic: NP3_F10
AREVTTATKGVFSWLAY

SEQ ID NO: 231 - protein, 15 aa, Artificial Sequence
  Synthetic: NP3_G1
TRLEGYGDYDYAMYY

SEQ ID NO: 232 - protein, 12 aa, Artificial Sequence
  Synthetic: NP3_G5
ARAPLLRSYFDY

SEQ ID NO: 233 - protein, 12 aa, Artificial Sequence
  Synthetic: NP3_G6
ARGSTYGNPLDY

SEQ ID NO: 234 - protein, 11 aa, Artificial Sequence
  Synthetic: NP3_G7
ARRPLFYAMDY

SEQ ID NO: 235 - protein, 11 aa, Artificial Sequence
  Synthetic: NP3_G8
ARSGDIYAMDY

SEQ ID NO: 236 - protein, 11 aa, Artificial Sequence
  Synthetic: NP3_G9
ARRPLFYAMDY

SEQ ID NO: 237 - protein, 12 aa, Artificial Sequence
  Synthetic: NP3_G12
ARRLFLRRYFDV

SEQ ID NO: 238 - protein, 11 aa, Artificial Sequence
  Synthetic: NP3_H2
ARAGEVYAKDY

SEQ ID NO: 239 - protein, 13 aa, Artificial Sequence
  Synthetic: NP3_H9
ARHYRYDEAWFVY

SEQ ID NO: 240 - protein, 14 aa, Artificial Sequence
  Synthetic: NP3_H12
ARTREIWGNVDFPY

SEQ ID NO: 241 - protein, 6 aa, Artificial Sequence
  Synthetic: NP3_A3
ENIYSN

SEQ ID NO: 242 - protein, 6 aa, Artificial Sequence
  Synthetic: NP3_A4
QDVSTA

SEQ ID NO: 243 - protein, 6 aa, Artificial Sequence
  Synthetic: NP3_A6
ENIYSN

SEQ ID NO: 244 - protein, 11 aa, Artificial Sequence
  Synthetic: NP3_A7
KSLLHSNGNTY

SEQ ID NO: 245 - protein, 10 aa, Artificial Sequence
  Synthetic: NP3_A8
KSVSASGYSY

SEQ ID NO: 246 - protein, 11 aa, Artificial Sequence
  Synthetic: NP3_A9
KSLLHSNGFTY

SEQ ID NO: 247 - protein, 10 aa, Artificial Sequence
  Synthetic: NP3_A10
QSVDYDGDSY

SEQ ID NO: 248 - protein, 6 aa, Artificial Sequence
  Synthetic: NP3_A11
ENIYSS

SEQ ID NO: 249 - protein, 11 aa, Artificial Sequence
  Synthetic: NP3_B1
QTLLDSDGKTY

SEQ ID NO: 250 - protein, 6 aa, Artificial Sequence
  Synthetic: NP3_B2
QGISNY

SEQ ID NO: 251 - protein, 10 aa, Artificial Sequence
  Synthetic: NP3_B3
QSVDYDGDSY

SEQ ID NO: 252 - protein, 5 aa, Artificial Sequence
  Synthetic: NP3_B4
SSVSY

SEQ ID NO: 253 - protein, 5 aa, Artificial Sequence
  Synthetic: NP3_B5
SSVNY

SEQ ID NO: 254 - protein, 6 aa, Artificial Sequence
  Synthetic: NP3_B8
QGISNY

SEQ ID NO: 255 - protein, 6 aa, Artificial Sequence
  Synthetic: NP3_B9
ENIYSN

SEQ ID NO: 256 - protein, 10 aa, Artificial Sequence
  Synthetic: NP3_B10
QSVDFDGKSY

SEQ ID NO: 257 - protein, 10 aa, Artificial Sequence
  Synthetic: NP3_B12
QSVDYDGYSY

SEQ ID NO: 258 - protein, 6 aa, Artificial Sequence
  Synthetic: NP3_C3
ENIYSN

SEQ ID NO: 259 - protein, 11 aa, Artificial Sequence
  Synthetic: NP3_C4
QSLLDSDGKTY

SEQ ID NO: 260 - protein, 6 aa, Artificial Sequence
  Synthetic: NP3_C5
ENIYSN

SEQ ID NO: 261 - protein, 10 aa, Artificial Sequence
  Synthetic: NP3_C6
QSVDYDGDSY

SEQ ID NO: 262 - protein, 10 aa, Artificial Sequence
  Synthetic: NP3_C8
QSVDYDGDSY

SEQ ID NO: 263 - protein, 10 aa, Artificial Sequence
  Synthetic: NP3_C9
QSVDYDGDSY

SEQ ID NO: 264 - protein, 6 aa, Artificial Sequence
  Synthetic: NP3_C10
SSSVSY

SEQ ID NO: 265 - protein, 7 aa, Artificial Sequence
  Synthetic: NP3_C11
SSVSSNY

SEQ ID NO: 266 - protein, 11 aa, Artificial Sequence
  Synthetic: NP3_D1
QSLLDSDGKTY

SEQ ID NO: 267 - protein, 10 aa, Artificial Sequence
  Synthetic: NP3_D4
QSVDYDGDSY

SEQ ID NO: 268 - protein, 6 aa, Artificial Sequence
  Synthetic: NP3_D5
QDVGTA

SEQ ID NO: 269 - protein, 6 aa, Artificial Sequence
  Synthetic: NP3_E2
QDVGSA

SEQ ID NO: 270 - protein, 6 aa, Artificial Sequence
  Synthetic: NP3_E3
ENIYSN

SEQ ID NO: 271 - protein, 6 aa, Artificial Sequence
  Synthetic: NP3_E4
QDISDY

SEQ ID NO: 272 - protein, 6 aa, Artificial Sequence
  Synthetic: NP3_E5
QGISNY

SEQ ID NO: 273 - protein, 10 aa, Artificial Sequence
  Synthetic: NP3_E6
QSVDYDGDSY

SEQ ID NO: 274 - protein, 6 aa, Artificial Sequence
  Synthetic: NP3_E11
QDVGSA

SEQ ID NO: 275 - protein, 6 aa, Artificial Sequence
  Synthetic: NP3_F4
ENIYSN

SEQ ID NO: 276 - protein, 10 aa, Artificial Sequence
  Synthetic: NP3_F7
QSVDYDGDSY

SEQ ID NO: 277 - protein, 7 aa, Artificial Sequence
  Synthetic: NP3_F8
SSVSSSY

SEQ ID NO: 278 - protein, 6 aa, Artificial Sequence
  Synthetic: NP3_F10
QDISDY

SEQ ID NO: 279 - protein, 10 aa, Artificial Sequence
  Synthetic: NP3_G1
KSVSASGYSY

SEQ ID NO: 280 - protein, 6 aa, Artificial Sequence
  Synthetic: NP3_G5
QDVSTA

SEQ ID NO: 281 - protein, 6 aa, Artificial Sequence
  Synthetic: NP3_G6
QGISNY

SEQ ID NO: 282 - protein, 6 aa, Artificial Sequence
  Synthetic: NP3_G7
ENIYSN

SEQ ID NO: 283 - protein, 10 aa, Artificial Sequence
  Synthetic: NP3_G8
QSVDYDGGSY

SEQ ID NO: 284 - protein, 10 aa, Artificial Sequence
  Synthetic: NP3_G9
QSVDYDGESY

SEQ ID NO: 285 - protein, 10 aa, Artificial Sequence
  Synthetic: NP3_G12
QSVDYDGDSY

SEQ ID NO: 286 - protein, 10 aa, Artificial Sequence
  Synthetic: NP3_H2
QSVDYDGDSY

SEQ ID NO: 287 - protein, 6 aa, Artificial Sequence
  Synthetic: NP3_H9
ENIYSN

SEQ ID NO: 288 - protein, 6 aa, Artificial Sequence
  Synthetic: NP3_H12
ENIYSD

SEQ ID NO: 289 - protein, 3 aa, Artificial Sequence
  Synthetic: NP3_A3
GAT

SEQ ID NO: 290 - protein, 3 aa, Artificial Sequence
  Synthetic: NP3_A4
SAS

SEQ ID NO: 291 - protein, 3 aa, Artificial Sequence
  Synthetic: NP3_A6
GAT

SEQ ID NO: 292 - protein, 3 aa, Artificial Sequence
  Synthetic: NP3_A7
RMS

SEQ ID NO: 293 - protein, 3 aa, Artificial Sequence
  Synthetic: NP3_A8
LAS

SEQ ID NO: 294 - protein, 3 aa, Artificial Sequence
  Synthetic: NP3_A9
QMS

SEQ ID NO: 295 - protein, 3 aa, Artificial Sequence
  Synthetic: NP3_A10
AAS

SEQ ID NO: 296 - protein, 3 aa, Artificial Sequence
  Synthetic: NP3_A11
TAT

SEQ ID NO: 297 - protein, 3 aa, Artificial Sequence
  Synthetic: NP3_B1
LVS

SEQ ID NO: 298 - protein, 3 aa, Artificial Sequence
  Synthetic: NP3_B2
FTS

SEQ ID NO: 299 - protein, 3 aa, Artificial Sequence
  Synthetic: NP3_B3
AAS

SEQ ID NO: 300 - protein, 3 aa, Artificial Sequence
  Synthetic: NP3_B4
DTS

SEQ ID NO: 301 - protein, 3 aa, Artificial Sequence
  Synthetic: NP3_B5
DTS

SEQ ID NO: 302 - protein, 3 aa, Artificial Sequence
  Synthetic: NP3_B8
YTS

SEQ ID NO: 303 - protein, 3 aa, Artificial Sequence
  Synthetic: NP3_B9
GAT

SEQ ID NO: 304 - protein, 3 aa, Artificial Sequence
  Synthetic: NP3_B10
VAS

SEQ ID NO: 305 - protein, 3 aa, Artificial Sequence
  Synthetic: NP3_B12
AAS

SEQ ID NO: 306 - protein, 3 aa, Artificial Sequence
  Synthetic: NP3_C3
VAT

SEQ ID NO: 307 - protein, 3 aa, Artificial Sequence
  Synthetic: NP3_C4
LVS

SEQ ID NO: 308 - protein, 3 aa, Artificial Sequence
  Synthetic: NP3_C5
GAT

SEQ ID NO: 309 - protein, 3 aa, Artificial Sequence
  Synthetic: NP3_C6
AAS

SEQ ID NO: 310 - protein, 3 aa, Artificial Sequence
  Synthetic: NP3_C8
AAS

SEQ ID NO: 311 - protein, 3 aa, Artificial Sequence
  Synthetic: NP3_C9
VAS

SEQ ID NO: 312 - protein, 3 aa, Artificial Sequence
  Synthetic: NP3_C10
DTS

SEQ ID NO: 313 - protein, 3 aa, Artificial Sequence
  Synthetic: NP3_C11
STS

SEQ ID NO: 314 - protein, 3 aa, Artificial Sequence
  Synthetic: NP3_D1
LVS

SEQ ID NO: 315 - protein, 3 aa, Artificial Sequence
  Synthetic: NP3_D4
AAS

SEQ ID NO: 316 - protein, 3 aa, Artificial Sequence
  Synthetic: NP3_D5
WAS

SEQ ID NO: 317 - protein, 3 aa, Artificial Sequence
  Synthetic: NP3_E2
WTS

SEQ ID NO: 318 - protein, 3 aa, Artificial Sequence
  Synthetic: NP3_E3
AAT

SEQ ID NO: 319 - protein, 3 aa, Artificial Sequence
  Synthetic: NP3_E4
YTS

SEQ ID NO: 320 - protein, 3 aa, Artificial Sequence
  Synthetic: NP3_E5
YTS

SEQ ID NO: 321 - protein, 3 aa, Artificial Sequence
  Synthetic: NP3_E6
AAS

SEQ ID NO: 322 - protein, 3 aa, Artificial Sequence
  Synthetic: NP3_E11
WTS

SEQ ID NO: 323 - protein, 3 aa, Artificial Sequence
  Synthetic: NP3_F4
AAT

SEQ ID NO: 324 - protein, 3 aa, Artificial Sequence
  Synthetic: NP3_F7
AAS

SEQ ID NO: 325 - protein, 3 aa, Artificial Sequence
  Synthetic: NP3_F8
RTS

SEQ ID NO: 326 - protein, 3 aa, Artificial Sequence
  Synthetic: NP3_F10
YTS

SEQ ID NO: 327 - protein, 3 aa, Artificial Sequence
  Synthetic: NP3_G1
LAS

SEQ ID NO: 328 - protein, 3 aa, Artificial Sequence
  Synthetic: NP3_G5
SAS

SEQ ID NO: 329 - protein, 3 aa, Artificial Sequence
  Synthetic: NP3_G6
FTS

SEQ ID NO: 330 - protein, 3 aa, Artificial Sequence
  Synthetic: NP3_G7
VAT

SEQ ID NO: 331 - protein, 3 aa, Artificial Sequence
  Synthetic: NP3_G8
AAS

SEQ ID NO: 332 - protein, 3 aa, Artificial Sequence
  Synthetic: NP3_G9
AAS

SEQ ID NO: 333 - protein, 3 aa, Artificial Sequence
  Synthetic: NP3_G12
AAS

SEQ ID NO: 334 - protein, 3 aa, Artificial Sequence
  Synthetic: NP3_H2
AAS

SEQ ID NO: 335 - protein, 3 aa, Artificial Sequence
  Synthetic: NP3_H9
AAT

SEQ ID NO: 336 - protein, 3 aa, Artificial Sequence
  Synthetic: NP3_H12
AAT

SEQ ID NO: 337 - protein, 9 aa, Artificial Sequence
  Synthetic: NP3_A3
QHFWGTPWT

SEQ ID NO: 338 - protein, 9 aa, Artificial Sequence
  Synthetic: NP3_A4
QQHYSNPLT

SEQ ID NO: 339 - protein, 9 aa, Artificial Sequence
  Synthetic: NP3_A6
QHFWGTPWT

SEQ ID NO: 340 - protein, 9 aa, Artificial Sequence
  Synthetic: NP3_A7
MQHLEYPLT

SEQ ID NO: 341 - protein, 9 aa, Artificial Sequence
  Synthetic: NP3_A8
QHSRELPLT

SEQ ID NO: 342 - protein, 7 aa, Artificial Sequence
  Synthetic: NP3_A9
AQNLELP

SEQ ID NO: 343 - protein, 9 aa, Artificial Sequence
  Synthetic: NP3_A10
QQSIEDPYT

SEQ ID NO: 344 - protein, 9 aa, Artificial Sequence
  Synthetic: NP3_A11
QHFWGFPWT

SEQ ID NO: 345 - protein, 9 aa, Artificial Sequence
  Synthetic: NP3_B1
WQGTHFPQT

SEQ ID NO: 346 - protein, 9 aa, Artificial Sequence
  Synthetic: NP3_B2
QQYSKLPFT

SEQ ID NO: 347 - protein, 9 aa, Artificial Sequence
  Synthetic: NP3_B3
QQSNEDPYT

SEQ ID NO: 348 - protein, 9 aa, Artificial Sequence
  Synthetic: NP3_B4
FQGSGYPLT

SEQ ID NO: 349 - protein, 9 aa, Artificial Sequence
  Synthetic: NP3_B5
HQRSSFPFT

SEQ ID NO: 350 - protein, 9 aa, Artificial Sequence
  Synthetic: NP3_B8
QQYNKLPYT

SEQ ID NO: 351 - protein, 9 aa, Artificial Sequence
  Synthetic: NP3_B9
QLFWGTPWT

SEQ ID NO: 352 - protein, 9 aa, Artificial Sequence
  Synthetic: NP3_B10
QQTNEDPLT

SEQ ID NO: 353 - protein, 9 aa, Artificial Sequence
  Synthetic: NP3_B12
QQSNADPFT

SEQ ID NO: 354 - protein, 9 aa, Artificial Sequence
  Synthetic: NP3_C3
QHFWGTPWT

SEQ ID NO: 355 - protein, 9 aa, Artificial Sequence
  Synthetic: NP3_C4
WQGTHFPRT

SEQ ID NO: 356 - protein, 9 aa, Artificial Sequence
  Synthetic: NP3_C5
QHFWGTPWT

SEQ ID NO: 357 - protein, 9 aa, Artificial Sequence
  Synthetic: NP3_C6
QQSNEDPWT

SEQ ID NO: 358 - protein, 9 aa, Artificial Sequence
  Synthetic: NP3_C8
QQSIGDPWT

SEQ ID NO: 359 - protein, 9 aa, Artificial Sequence
  Synthetic: NP3_C9
QQTNEDPLT

SEQ ID NO: 360 - protein, 9 aa, Artificial Sequence
  Synthetic: NP3_C10
QQWSGYPYT

SEQ ID NO: 361 - protein, 9 aa, Artificial Sequence
  Synthetic: NP3_C11
HQFHRSPRT

SEQ ID NO: 362 - protein, 9 aa, Artificial Sequence
  Synthetic: NP3_D1
WQGTHFPQT

SEQ ID NO: 363 - protein, 9 aa, Artificial Sequence
  Synthetic: NP3_D4
QQSIEDPYT

SEQ ID NO: 364 - protein, 9 aa, Artificial Sequence
  Synthetic: NP3_D5
HQYSSYPYT

SEQ ID NO: 365 - protein, 9 aa, Artificial Sequence
  Synthetic: NP3_E2
QQYSRYPLT

SEQ ID NO: 366 - protein, 9 aa, Artificial Sequence
  Synthetic: NP3_E3
QHFWGTPWT

SEQ ID NO: 367 - protein, 9 aa, Artificial Sequence
  Synthetic: NP3_E4
QQYSKLPYT

SEQ ID NO: 368 - protein, 9 aa, Artificial Sequence
  Synthetic: NP3_E5
QQYSKLPFT

SEQ ID NO: 369 - protein, 9 aa, Artificial Sequence
  Synthetic: NP3_E6
QQSNEDPFT

SEQ ID NO: 370 - protein, 9 aa, Artificial Sequence
  Synthetic: NP3_E11
HQYSSYPYT

SEQ ID NO: 371 - protein, 9 aa, Artificial Sequence
  Synthetic: NP3_F4
QHFWGFPRT

SEQ ID NO: 372 - protein, 9 aa, Artificial Sequence
  Synthetic: NP3_F7
QQSNEDPWT

SEQ ID NO: 373 - protein, 9 aa, Artificial Sequence
  Synthetic: NP3_F8
QQWSGYPFT

SEQ ID NO: 374 - protein, 9 aa, Artificial Sequence
  Synthetic: NP3_F10
QQYSKLPWT

SEQ ID NO: 375 - protein, 9 aa, Artificial Sequence
  Synthetic: NP3_G1
QHRREIPLT

SEQ ID NO: 376 - protein, 9 aa, Artificial Sequence
  Synthetic: NP3_G5
QQYYSTPFT

SEQ ID NO: 377 - protein, 9 aa, Artificial Sequence
  Synthetic: NP3_G6
QQYSKLPFT

SEQ ID NO: 378 - protein, 10 aa, Artificial Sequence
  Synthetic: NP3_G7
CQHFWGTPWT

SEQ ID NO: 379 - protein, 9 aa, Artificial Sequence
  Synthetic: NP3_G8
QQSNEDPWT

SEQ ID NO: 380 - protein, 9 aa, Artificial Sequence
  Synthetic: NP3_G9
QQSYEDPWT

SEQ ID NO: 381 - protein, 9 aa, Artificial Sequence
  Synthetic: NP3_G12
QQSNEDPFT

SEQ ID NO: 382 - protein, 9 aa, Artificial Sequence
  Synthetic: NP3_H2
QQSNEDPWT

SEQ ID NO: 383 - protein, 9 aa, Artificial Sequence
  Synthetic: NP3_H9
QHFWGTPWT

SEQ ID NO: 384 - protein, 9 aa, Artificial Sequence
  Synthetic: NP3_H12
QHFWGTPWT

SEQ ID NO: 385-392 - skipped (8 entries)

SEQ ID NO: 393 - DNA, 360 bp, Artificial Sequence
  Synthetic: NP3_A3
gatgtgcagctgcaggagtctggacctgagctgaagaagcctggagagacagtcaagatctcctgcaaggcttctggttataccttcacagactatgcaattcactgggtgaagcaggctccaggaaagggtttaaagtggatgggctggataaacactgagactggcgagccaaaatatgcagaggacttcaagggacggtttgccttctctttggaaacctctgtcagcactgcctatttgcagagcaacaacctcaaaaacgaggacacggctacatatttctgtgctagacactataggtacgacgaggcctggtttgcttactggggccgagggactctggtcacagtctcctca

SEQ ID NO: 394 - DNA, 357 bp, Artificial Sequence
  Synthetic: NP3_A4
caggttactctgaaagagtctggccccgggatattgaagccctcacagaccctcagtctgacttgttctttctctgggttttcactgagcacttctggtatgggtgtaggctggattcgtcagccttcagggaagggtctggagtggctggcacacatttggtgggatgatgataagtaccataacccatccctgaagagccagctcacaatctccaaggatacctccagaaaccaggtattcctcaagatcaccagtgtggacactgcagatactgccacttactactgtgctcgaagcggatataggggaaactttgactactggggccaaggcaccacggtcaccgtctcctca

SEQ ID NO: 395 - DNA, 360 bp, Artificial Sequence
  Synthetic: NP3_A6
cagatccagttggtgcagtctggacctgagttgaagaagcctggagagacagtcaagatcgcctgtaaggcttctggttataccttcacagactattcaatgaactgggtgaagcaggctccaggaaagggtttaaagtggatgggctggataaacactgagactggcgagccaacatatgaagatgacttcaagggacggtttgccttctctttggaaacctctgccagcactgcctatttgcagatcaccaacctcaaaaatgaggacacggctacatatttctgtgttagagggtataggtacgacgaggcctggtttgtttactggggccaagggactctggtcactgtctctgca

SEQ ID NO: 396 - DNA, 351 bp, Artificial Sequence
  Synthetic: NP3_A7
cagatccagttggtgcagtctggggcagagcttgtgaagccaggggcctcagtcaagttgtcctgcacagcttctggcttcaacattaaagacacctatatccactgggtgagacagaggcctgaacagggcctggagtggattggaaggattgatcctgcgaatggtgatactaaatatgacccgaagttccaggacaaggccactttaatagcagacacatcctccaacacagcctacctgcagctcggcagcctgacatctgaggacactgccgtctattactgtgctagatatcgtatttactactttgactactggggccaaggcaccactctcacagtctcctca

SEQ ID NO: 397 - DNA, 366 bp, Artificial Sequence
  Synthetic: NP3_A8
caggtgcagctgcagcagtctggggctgaactggtgaagcctggggcttcagtgaagttgtcctgcaaggcttcaggctacaccttcaccagctactatatgtactgggtgaaacagaggcctggacaaggccttgagtggattggagagattaatcctagcaatggtggttttagcttcaatgagaagttcaagagtaaggccacactgactgtagacaagtcctccagcacagcatacatgcaactcaccagcctgacatctgaggactctgcggtctattactgtacaagattagaggggtatggtgactccgactatgctatgatctactggggtcaaggaaccacggtcaccgtctcctca

SEQ ID NO: 398 - DNA, 360 bp, Artificial Sequence
  Synthetic: NP3_A9
gaggtgcagcttgttgagtctggagctgagctggtgaagcctggggcttcagtgaagatatcctgcaaggcttctggctacacattcactgactacaacatggactgggtgaagcagagccatggaaagagccttgagtggattggagatattaatcctgactatggtagtactagttacaaccagaagttcaggggaaaggccactttgactgtagacaagtcctccagcacagccaacatggagctccgcagcctgacatctgaggacactgcagtctattactgtgcaaggggggattacgacgggggctatggtatggactactggggtcaaggaacctcagtcaccgtctcctca

SEQ ID NO: 399 - DNA, 354 bp, Artificial Sequence
  Synthetic: NP3_A10
caggtgcagctgcagcagtctggacctagcctcgtaaaaccgtctcagactctgtccttcacctgttctgtcactggcgactccgtcaccagtggttactggaactggatccggaaattcccagggaataaacttgagttcatggggtacataagttacagtggtagcacttactacagtccatctctcaaaagtcgaatctccatcattcgcgacacatccaagaaccggtactacctgcagttgaactctgtgactactgaggacacagccacatatttctgtgcaagggggctacggtttttctatgccatggattactggggtcaaggaaccactctcacagtctcctca

SEQ ID NO: 400 - DNA, 360 bp, Artificial Sequence
  Synthetic: NP3_A11
gaggttcagctgcaggagtctggacctgagctgaagaagcctggagagacagtcaagatctcctgcaaggtttctgggtataccttcacaaactatggaatgaactgggtgaagcaggctccaggcaagggtttaaagtggatgggctggataaacacctacactggagagccaaaatatggtgatgacttcaagggacggtttgccctctctttggagacctctgccggtactgcctatttgcggattaataatctcaaaaatgaggacacggctacatatttctgtgcaagatcccatggtaactacgagaaccactttgactactggggccaaggcaccactctcaccgtctcctca

SEQ ID NO: 401 - DNA, 339 bp, Artificial Sequence
  Synthetic: NP3_B1
caggtgcagctgcagcagtctggacctgagctggtgaagcctgggacttcagtgacggtatactgcaaggcttctggttatacattcactacctacaacatttactgggtgaagcagagccatggaaagagccttgagtggattggatatattgatccttacaatggtggtactagctacaaccagaagttcaagggcaaggccacattgactgttgacaagtcctccagcgcagcctacatgcatctcaacagcctgacatctgaggactctgcagtctattactgtgccctcccatgggactactggggccaaggcaccacggtcaccgtctcctca

SEQ ID NO: 402 - DNA, 357 bp, Artificial Sequence
  Synthetic: NP3_B2
cagatccagttggtgcagtctggggggggcttagtgaagcctggagggtccctgaaactctcctgtgcagcctctggattcactttcagtgactattacatgtcttgggttcgccagactccggaaaagcggctggagtgggtcgcaaccattagtgatggtggtagttacacctactatccagacagtgtgaaggggcgattcaccatctccagagacaatgccaagaacaacctgtacctgcacatgagcagtctgaagtctgaggacacagccatgtattactgtgcaagagggtctacctatggtaaccctcttgactactggggccaaggcaccactctcacagtctcctca

SEQ ID NO: 403 - DNA, 357 bp, Artificial Sequence
  Synthetic: NP3_B3
gaggttcagctgcagcagtctgggactgtgctggcaaggcctggggcttccgtgaagatgtcctgcaaggcttctggctccagctttaccacctactggatgcactgggtaaaacagaggcctggacagggtctagaatggattggtgctatttatcctggaaatagtgatactagctacaaccaaaagttcaagggcaaggccaaactgactgcagtcacatccgccagcactgcctacatggagctcagcagcctgacaaatgaggactctgcggtctattactgtacaagaaaggaattatttgggtacgggtttgactactggggccaaggcaccactctcacagtctcctca

SEQ ID NO: 404 - DNA, 363 bp, Artificial Sequence
  Synthetic: NP3_B4
gaggtgcagcttgttgagtctggaggaggcttggtgcaacctggaggatccgtgaaactctcctgtttagcctctggatttaccttcagtgactactacatgtcttgggtccgccagtctccagagaaggggcttgagtgggttgctgaaattagattggaatctgataattatgcaacacattatgcggagtctgtgaaagggaagttcaccatctcaagagatgattccaaaagtcgtctctatctgaaaatgaacagcttaaggggtgaagacactggaatttattactgtgcgtttgatgtttactacgggggggctatggactactggggtcaaggaaccacggtcaccgtctcctca

SEQ ID NO: 405 - DNA, 360 bp, Artificial Sequence
  Synthetic: NP3_B5
caggtgcagctgcagcagtctggggctgagctggtgaggcctggggcttcagtgaagctgtcctgcaaggcttctggctacacgttcaccagctactggatgaactgggttaaacagaggcctgaacaaggccttgcgtggattggaaggattgatccttacgataatgcaactcactacaatcacaagttcaaggacaaggccatattgactgtagacaaatcctccagcacagcctacatggaactcagaggcctgacatctgaggactctgcggtctattactgtgcaatctactatgattacgacgactatattatggactactggggtcaaggaacctcagtcaccgtctcctca

SEQ ID NO: 406 - DNA, 357 bp, Artificial Sequence
  Synthetic: NP3_B8
gatgtgcagctgcaggagtctgggggaggcttagtgaagcctggagggtccctgaaactctcctgtgcagcctctggattcactttcagtgactattacatgtattgggttcgccagactccggaaaggaggctggagtgggtcgcaaccattagtgatggtggtagttacacctactatccagacagtgtgaaggggcgattcaccagctccagagacaatgccaagaacaacctgtacctgcaaatgagcagtctgaagtctgaggacacagccatgtattactgtacaagaggggatgactacggtggtaggcttgcttactggggccaagggactctggtcactgtctctgca

SEQ ID NO: 407 - DNA, 363 bp, Artificial Sequence
  Synthetic: NP3_B9
gaggttcagctgcagcagtctggacctgagttgaagaagcctggagagacagtcaagatctcctgcaagacttctggatataccttcacaaactgtggcatgcactgggtgaagcaggctccaggaaagggtttaaagtggatgggctggataaacacctacactggagagccaacatatgctgatgacttcaagggacggtttgccttctctttggaaacctctgtcaacactgcctatttgcagatcaacaacctcaaaagtgaggacacggcttcatatttctgtgcaagaagggatgatgtttactacgtcgccgggtttgactactggggccaaggcaccactctcacagtctcctca

SEQ ID NO: 408 - DNA, 345 bp, Artificial Sequence
  Synthetic: NP3_B10
cagatccagttggtgcagtctggacctgaactgaagaagcctggagagacagtcaagatctcctgcaaggcttctggttataccttcacagactattcaatgcactgggtgaagcaggctccaggaaaggatttaaagtgggtgggctggataaacactgagactggtgagccaacatatgcagatgacttcaagggacggtttgccttctctttggagacctctgccaccactgcctatttgcaaatcaacaacctcaaaaatgaggacacggctacatatttctgtgctagaggggcggctacgagatactggggccaaggcaccactctcacagtctcctca

SEQ ID NO: 409 - DNA, 369 bp, Artificial Sequence
  Synthetic: NP3_B12
gaggttcagctgcagcagtctggagctgagctgatgaagcctggggcctcagtgaagatatcctgcaaggctactggctacacattcagtagctactggatagagtgggtaaagcagaggcctggacatggccttgagtggattggagagttttttcctggtagtggtaatactaactacaatgagaagttcaagggcaaggccacattcactgcagatacatcctccaacacagcctacatgcaactcagcagcctgacatctgaggactctgccgtctattactgtgcgagaaggggcctctatgatggttactacgttcggtacttcgatgtctggggcgcagggaccacggtcaccgtctcctca

SEQ ID NO: 410 - DNA, 360 bp, Artificial Sequence
  Synthetic: NP3_C3
cagatccagttggtgcagtctggacctgagctgaggaagcctggagagacagtcaagatctcctgcaaggcttctgggtatatcttcacaaactatggaatgaactgggtgaagcaggctccaggaaagggtttaaagtggatgggctggataaacacttacactggagagccaacatatgctggtgacttcaagggacggtttgccttctctttggaaacctctgccagcactgcctatttgcagatcaacaacctcaaaaatgaggactcggctacatatttctgtgcaagatcccatgataactacgataaccactttgactactggggccaaggcaccactctcacagtctcctca

SEQ ID NO: 411 - DNA, 339 bp, Artificial Sequence
  Synthetic: NP3_C4
cagatccagttggtgcagtctggacctgagctggtgaagcctggggcttcagtgaagatatcctgcaaggcttctggttactcattcactaactactacatgcactgggtgaggcaaagccatgtaaagagccttgagtggattggacgtattaatccttacagtggtgctactagctacaaccagcatttcaaggacaaggccagcttgactgtcgataagtcctccagcacagcctacatggagctccacagcctgacatctgaggactctgcagtctattactgtgcagacaatggttacctttggggtcaaggaacctcagtcaccgtctcctca

SEQ ID NO: 412 - DNA, 363 bp, Artificial Sequence
  Synthetic: NP3_C5
cagatccagttggtgcagtctggacctgagctgaagacgcctggagagacagtcaagatctcctgcaaggcttctgggtataccttcacaaactgtggaatgcactgggtgaagcaggctccaggaaagggtttaaagtggatgggctggataaacacctacactggggagccaatatatgctgatgacttcaagagacggtttgccttctctttggaaacctctgccagcactgcctatttgcagatcaacaacctcaataatgaggacacggcttcatatttctgtgcaagaagggatgatgtttactacgtcgccgggtttgactactggggccaaggcaccacggtcaccgtcttctca

SEQ ID NO: 413 - DNA, 369 bp, Artificial Sequence
  Synthetic: NP3_C6
caggtgcagctgcagcagtctgggcctgagctggtgaagcctggggcttcagtgaagatgtcctgcaaggcttctggatacacattcactgactatgttataaactgggtgaaggagagaactggacagggccttgagtggattggagagatttatcctggaagtggtgatacttactacaatgagaagttcaagggcaaagccacactgactgctgacaaatcctccaacacagcctacatgcacctcagcagcctgacatctgaggactctgcggtctttttctgtgcaagaaggaaattctatgatggtaactacgtcaggtacttcgatgtctggggcgcagggaccactctcacagtctcctca

SEQ ID NO: 414 - DNA, 354 bp, Artificial Sequence
  Synthetic: NP3_C8
gaggttcagctgcagcagtctggggctgaactggcaagacctggggcctcagtaaagatgtcctgcaaggcttctggctacacctttactagctacacgatgcactgggtaaaacagaggcctggacagggtctggaatggcttggatactttaatcctagcagtggttatactaattacgttcagaggttcagggacaaggccacattgaccgcagacaaatcctccagcacagcctacatgcaactgagcagcctgacatctgaggactctgcagtctattactgtgcaagagcgggcgagggctatgctatggactactggggtcaaggaacctcagtcaccgtctcctca

SEQ ID NO: 415 - DNA, 345 bp, Artificial Sequence
  Synthetic: NP3_C9
cagatccagttggtgcagtctggacctgaactgaagaagcctggagagacagtcaagatctcctgcaaggcttctggttataccttcacagactattcaatacactgggtgaagcaggctccaggaaaggatttaaagtgggtgggctggataaacactgagactggtgagccaacatatgcagatgacttcgagggacggtttgccttctctttggaaacctctgccagcactgccttcttgcagatcaacaacctcaaaaatgaggacacggctacatatttctgtagtagaggggcggctacggggttctggggccaaggcaccactctcacagtctcctca

SEQ ID NO: 416 - DNA, 357 bp, Artificial Sequence
  Synthetic: NP3_C10
caggttactctgaaagagtctggccctgggatattgaagccctcacagaccctcagtctgacttgttctttctctgggttttcactgagcacatctggtttgggtgtaggctggattcgtcagccttcagggaagggtctggagtggctggcacacatttggtgggatgacgataagtactataacccatccctgaagagccagctcacaatctccaaggaaacctccagaaaccaggtattcctcaagatcagcagtgtggacagtgcagatactgccacttactactgtgctcgaaccgtttaccacggggctatggactactggggtcaaggaacctcagtcaccgtctcctca

SEQ ID NO: 417 - DNA, 363 bp, Artificial Sequence
  Synthetic: NP3_C11
gaggtgcagcttgttgagtctgggggaggcttagtgaggcctggagggtccctgaaactctcctgtgcagcttctggattcactttcagtgactattacatgtattgggttcgccagactccggaaaagaggctggagtgggtcgcaaccattagtgatggtggtggatacaattactatccagacagtgtgaaggggcgattcaccgtctccagagacaatgccaagaacaacctgtacctgcaaatgagcagtctgaagtctgaggacacagccatgtattactgtgtaagagataagtacgatggttgggggtctgatttcgatgtctggggcgcagggaccacggtcaccgtctcctca

SEQ ID NO: 418 - DNA, 339 bp, Artificial Sequence
  Synthetic: NP3_D1
gatgtgcagctgcaggagtctggacctgagctggcgaagcctggggcttcagtgaaggtatcctgcaaggcttctggttatacattcactagctacaacatatactgggtgaagcagagccatggaaagagccttgagtggattggatatattgatccttacaatggtggtactagctacaaccagaagttcaagggcaaggccacattgactgttgacaagtcctccagcacagcctacatgcatctcaacagcctgacatctgaggactctgcagtctattactgtgccctcccatgggactactggggccaaggcacctcagtcaccgtctcctca

SEQ ID NO: 419 - DNA, 354 bp, Artificial Sequence
  Synthetic: NP3_D4
gaggtgcagcttgttgagtctggacctagcctcgtaaaaccttctcagactctgtccctcacctgttctgtcactggcgactccatcaccagtggttactggaactggattcggaaattcccagggaataaatttgagttcatggggtacatgaggtacagtggtagcacttacttcagtccatctctcaaaagtcgaatctccatcactcgagacacatccaagaaccactactacctgcagttgagttctgtgactactgaggacacagccacatattactgtgcaagggggctaaggtttttctatgctatggactactggggtcaaggaacctcagtcaccgtctcctca

SEQ ID NO: 420 - DNA, 348 bp, Artificial Sequence
  Synthetic: NP3_D5
gatgtgcagctgcaggagtctgggcctgagctggtgaggcctggggtctcagtgaagatttcctgcaagggttccggctacacattcactgattatgttatgcactgggtgaagcagagtcatgcaaagagtctagagtggattggagttattagtacttactctggtaatacaaactacaaccagaagtttaaggggaaggccacaatgactgtagacaaattctccaacacagcctatatggaacttgccagattgacatctgaagattctgccatctattactgtacaagggggactggggactttgactactggggccaaggcaccactctcacagtctcctca

SEQ ID NO: 421 - DNA, 351 bp, Artificial Sequence
  Synthetic: NP3_E2
gaggttcagctgcagcagtctgggcctgaggtggtgaggcctggggtctcagtgaagatttcctgcaagggttccggctacacattcactgattatgctctgcactgggtcaagcagagtcatacaaagagtctagagtggattggagttattagtacgtacagtggtactacaaactacaaccagaactttaagggcaaggccacaatggctgtagacaaatcctccagcacagcctatatggaacttgccagattgacatctgaggattctgccatctattactgtgcaagtgggggtaactacggtttggactactggggtcaaggaacctcagtcaccgtctcctca

SEQ ID NO: 422 - DNA, 357 bp, Artificial Sequence
  Synthetic: NP3_E3
cagatccagttggtgcagtctggacctgacctgaagaagcctggagagacagtcaagatctcctgcaaggcttctggttataccttcacagactattcaatgcactgggtgaagcaggttccaggaaagggtttaaagtggatgggctggataaacactgagactgataaaccaacatatgcagatgacttcaagggacggtttgccttctctttggaaacctctgccagcactgcctattcgcagatcaacaacctcaaaaatgaggacacggctacatatttctgtgctagacactataaatacgacgaggcctggtcaccttactggggccaagggactctggtcactgtctct

SEQ ID NO: 423 - DNA, 357 bp, Artificial Sequence
  Synthetic: NP3_E4
gaggtgcagcttgttgagtctggacctggcctggtggcgccctcacagagcctgtccatcacatgcactgtctcagggttctcattaaccagctatggtgtaagctgggttcgccagcctccaggaaagggtctggagtggctgggagtaatatggggtgacgggagcacaaattatcattcagctctcatatccagactgagcatcaacaaggataactccaagagccaggttttcttaagactgaacagtctgcaaactgatgacacagccacgtactactgtgccaaagaggatgatggttacctgttctactttgactactggggccaaggcaccactctcacagtctcctca

SEQ ID NO: 424 - DNA, 357 bp, Artificial Sequence
  Synthetic: NP3_E5
gaagtgcagctggtggagtctgggggaggcttagtgaagcctggagggtccctgaaactctcctgtgcagcctctggattcactttcagtgactattacatgtcttgggttcgccagactccggaaaagaggctggagtgggtcgcaaccattagtgatggtggtaattacacctactatccagacagtgtgaaggggcgattcaccatctccagagacaatgccaagaacaacctgtacctgcaaatgagcagtctgaagtctgaggacacagccatgtattactgtgcaagagggtctacctatggtaaccctcttgaccactggggccaaggcaccactctcacagtctcctca

SEQ ID NO: 425 - DNA, 357 bp, Artificial Sequence
  Synthetic: NP3_E6
gaggttcagctgcagcagtctggagctgagctgatgaggcctggggcctcagtgaagatatcctgcaaggctactggctacacattcagttcctactggatagagtgggtaaagcagaggcctggacatggccttgagtggattggagagattttacctggaagtgataatactaactacaatgagaagttcaagggcaaggccacactcactgcagatacatcctccaacacagcctacatgcatctcagcagcctgacatctgaggactctgccgtctattcctgtgcaaggcgattgtttctacgacgttacttcgatgtctggggcgcagggaccacggtcaccgtctcctca

SEQ ID NO: 426 - DNA, 348 bp, Artificial Sequence
  Synthetic: NP3_E11
gaggttcagctgcagcagtctgggcctgagctggtgaggcctggggtctcagtgaagatttcctgcaagggttccggctacacattcactgattatgttatgcactgggtgaagcagagtcatgcaaagagtctagagtggattggagttattagtacttactctggtaatacaaactacaaccagaagtttaagggcaaggccacaatgactgtagacaaatcctccaacacagtctatatggaacttgccagattgacatctgaggattctgccatctattactgtacaagggggactggggactttgactactggggccaaggcaccactctcacagtctcctca

SEQ ID NO: 427 - DNA, 366 bp, Artificial Sequence
  Synthetic: NP3_F4
gaggtgcagcttgttgagtctggacctgagctgaagaagcctggagagacagtcaagatcccctgcaagacttctgggtataccttcacaaactatggaatgaactgggtgaagcaggctccaggaaagggtttaaagtggatgggctggattaacaccaacactggagagccgacatatgctgaggatttcaagggacggtttgccttctctttggaaacctctgccagcactgcctatttgcagatcagcaacctcaaaaatgaggacacggctacatttttctgtgcaagaggatccatctatgatggttactacgaggggctttactactggggccaaggcaccactctcacagtctcctca

SEQ ID NO: 428 - DNA, 354 bp, Artificial Sequence
  Synthetic: NP3_F7
gaggttcagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggaaatggtgatacttactacaatggtaagttcatgggtaaagccacactgactgcagacaaattttccagcacagcctacatgcagctcagcggcctaacttctgaggactctgcggtctatttctgtgcaagaaggccacttttctatgctatggactactggggtcaaggaacctcagtcaccgtctcctca

SEQ ID NO: 429 - DNA, 366 bp, Artificial Sequence
  Synthetic: NP3_F8
caggtgcagctgcagcagtctggacatggcctggtggcgccctcacagagcctgtccatcacatgcaccgtctcagggttctcattaagtagctatggtgtacattgggttcgccagcctccaggaaagggtctggagtggctggcagtgatatggagtgatggaaatacaatgtataattcagttctcaagtccagactgaacatcagcagggactactccaagagccaagttttcttaaaagggaacagtctccaaactgatgacacagccatgtactactgtgccagaaattccggctacaatgattacgacggatattatttcgatgtctggggcgcagggacctcagtcaccgtctcctca

SEQ ID NO: 430 - DNA, 369 bp, Artificial Sequence
  Synthetic: NP3_F10
gaggttcagctgcagcagtctggggctgagcttgtggagcctggggctccagtgaagctgtcctgcaaggctttcggctacaccttcaccagctactggatgaactgggtgaagcagaggcctggacgaggcctcgagtggattggaaggattgatccctccgatagtgaaactcactacaatcaaaacttcaaggacaaggccgcactgactgttgacagatcctccagcacagcctacatccatctcagcagcctgacatctgaggactctgcggtctattattgtgcaagagaggtgactacggctacgaagggggttttttcctggcttgcttattggggccaagggactctggtcactgtctct

SEQ ID NO: 431 - DNA, 366 bp, Artificial Sequence
  Synthetic: NP3_G1
gaggtgcagcttgttgagtctggggctgagctggtgaagcctggggcttcagtgaaattgtcctgcaaggcttctggctacaccttcaccagctactatatgtactgggtgcagctgaggcctggacaaggccttgagtggattggagagattaatcccagcaatggtggtactaacttcaatgagaagttcaagagcaaggccacactgactgtagacaagtcctccagcacagcatacatgcaactcagcagcctgacatctgaggactctgcggtctattactgtacaagattagaagggtatggtgactacgactatgctatgtactactggggtcaaggaacctcagtcaccgtctcctca

SEQ ID NO: 432 - DNA, 357 bp, Artificial Sequence
  Synthetic: NP3_G5
gaggtgcagcttgttgagtctggacctgagctgaagaagcctggagagccagtcaggatctcctgcaaggcttctgggtatacttttacagactatggaatgaactgggtgaagcaggctccaggaaagggtttaaagtggatgggctggattaacacctacactggagagccaacatatgctgatgacttcaagggccggtttgccttctctttggaaacctctgccagcactgcctttttgcagatcaccaacctcaaaaatgaggacacggctacatatttctgtgcaagagccccattactacggtcctactttgactactggggccaaggcacctcagtcaccgtctcctca

SEQ ID NO: 433 - DNA, 357 bp, Artificial Sequence
  Synthetic: NP3_G6
gaggtgcagcttgttgagtctgggggaggcttagtgaagcctggagggtccctgaaactctcctgtgcagcctccggattcactttcagtgactattacatgtcttgggttcgccagactccggaaaagcggctggagtgggtcgcaaccattagtgatggtggtagttacacctactacccagacagtgtgaaggggcgattcaccatctccagagacaatgccaagaacaacctgtacctgcacatgagcagtctgaagtctgaggacacagccatgtattactgtgcaagagggtctacctatggtaaccctctagactactggggccaaggcacctcagtcaccgtctcctca

SEQ ID NO: 434 - DNA, 354 bp, Artificial Sequence
  Synthetic: NP3_G7
cagatccagttggtgcagtctggggctgagctggtgaggcctgggtcctcagtgaagatttcctgcaaggcctctggctatgcattcagtagctactggatgaactgggtgaagcagaggcctggacagggtcttgagtggatcggacagatttatcctggaaatggtgatacttactacaatggtaagttcatgggtaaagccacactgactgcagacaaattttccagcacagcctacatgcagctcagcagcctaacttctgaggactctacggtctatttctgtgcaagaaggccacttttctatgctatggactactggggtcaaggaacctcagtcaccgtctcctca

SEQ ID NO: 435 - DNA, 354 bp, Artificial Sequence
  Synthetic: NP3_G8
caggtgcagctgcagcagtctggacctgagctgaagaagcctggagagacagtcaagatctcctgcaaggcttctggttataccttcaccgactattcaatgcactgggtgaagcaggctccaggaaagggtttaaagtggttgggctggaaaaacactgagagtggtgaggaaacatttgcagatgacttcaagggacggtttgacttctctttggaaacctctgccagcactgtctatttgcagatcaaaaacctcaaaaatgaagacacggctgcatatttctgtgctagatcgggggacatctatgctatggactactggggtcaaggaaccacggtcaccgtctcctca

SEQ ID NO: 436 - DNA, 354 bp, Artificial Sequence
  Synthetic: NP3_G9
gaggttcagctgcagcagtctggggctgagctggtgaggcctgggtcctcagtgaagatttcctgcaaggcttctggctatgcgttcagtaactactggatgaactgggtgaggcagaggcctggacagggtcttgagtggattggacagatttatcctggaaatgatgatacttactacaatggaaagttcaagggtaaagtcacactgactgcagacaagtcctccaccacagcctacatccagctcatcagcctaacatctgaggactctgcggtctatttctgtgcaagaaggccacttttctatgctatggactactggggtcagggaacctcagtcaccgtctcctca

SEQ ID NO: 437 - DNA, 357 bp, Artificial Sequence
  Synthetic: NP3_G12
gaggctcagctgcagcagtctggagctgagccgatgaagcctggggcctcagtgaagatatcctgcaagactactggctacacattcactagcgactggatagagtgggtacaacagaggcctggacatggccttgagtggattggagagattttacctgggagtggtaatactaactacaatgagaacttcaagggcaaggccacactcactgcagatacatcctccaacacagcctacatgcatctcagcagcctgacatctgaggactctgccgtctattactgtgcaaggcgattgtttctacgacggtacttcgatgtctggggcgcagggaccacggtcaccgtctcctca

SEQ ID NO: 438 - DNA, 354 bp, Artificial Sequence
  Synthetic: NP3_H2
gaggttcagctgcagcagtctggggctgaactggcaagacctggggcctcagtgaagatgtcctgcaaggcttctggctacatctttactagctacacgatccactggataaaacagaggcctggacagggtctggaatggattggatactttaatcctagcagtgattattttagttacattcagatcttcaaggacaaggccacattgactgcagacaaattctccagcacagcctacatgcaactgagcagcctgacatctgaggactctgcagtctattactgtgcaagagcgggcgaggtctatgctaaggactactggggtcaaggaacctcagtcaccgtctcctca

SEQ ID NO: 439 - DNA, 396 bp, Artificial Sequence
  Synthetic: NP3_H9
cagatccagttggtgcagtctggacctgagctgaagaagcctggagagacagtcaagatctcctgcaaggcttctggttataccttcacagactatccaatacactgggtgaagcaggctccaggaaagggtttaaagtggatgggctggataaacgctgagactggtgagccaacatatgcagatgacttcaagggacggtttgtcgtctctttggaaacctctgccagtactgcctatttacagatcaacaacctcaaaaatgaggacacggctacttttttctgtgctagacactataggtacgacgaggcctggtttgtttattggggccaagggactctggtcactgtctctgcagctagcaccaagggcccatcggtcaccgtctcctca

SEQ ID NO: 440 - DNA, 360 bp, Artificial Sequence
  Synthetic: NP3_H12
cagatccagttggtacagtctggacctgagctgaagaagcctggagagacagtcaagatctcctgcaaggcttctgggtatatgttcacaaactatggaatgaactgggtgaaacaggctccaggaaagggtttaaagtggatgggctggataaacacctacactggagagccaacatatgctgatgacttcaagggacggtttgccttctctttggatatttctggcagtactgcctatttgcagatcaacaacctcaaaaatgaggacatggctacatatttctgtgcgagaacgagggaaatatggggtaacgtcgattttccttactggggccaagggactctggtcactgtctct

SEQ ID NO: 441 - DNA, 321 bp, Artificial Sequence
  Synthetic: NP3_A3
gacattgtgatgactcagtctccagcctccctatctgtatctgtgggagaaactgtcaccatcacatgtcgatcaagtgagaatatttacagtaatttagcgtggtttcaacagaaacagggaaaatctcctcagctcctggtctatggtgcaacagacttagcagatggtgtgccatcaaggttcagtggcagtggatcgggcacacagtattccctcaagatcaacagcctgcagtctgaagattttgggacttattactgtcaacatttttggggcactccgtggacgttcggtggagggaccaagctggaaatcaaa

SEQ ID NO: 442 - DNA, 321 bp, Artificial Sequence
  Synthetic: NP3_A4
gacattgtgatgacccagtctcacaaattcatgtccacatcagtaggagacagggtcagcatcacctgcaaggccagtcaggatgtgagtactgctgtagcctggtatcaacagaaaccaggacaatctcctaaactactgatttattcggcatcccaccggtacactggagtccctgatcgcttcactggcagtggatctgggacggatttcactttcaccatcagcagtgtgcaggctgaagacctggcagtttattactgtcagcaacattatagtaatccgctcacgttcggtgctgggaccaagctggaaatcaaa

SEQ ID NO: 443 - DNA, 321 bp, Artificial Sequence
  Synthetic: NP3_A6
gacatccagatgaaccagtctccatcctccctatctgtatctgtgggagaaactgtcaccatcacatgtcgaacaagtgagaatatttacagtaatttagcatggtatcagcagaaacagggaaaatctcctcagctcctggtctatggtgcaacagacttagcagatggtgtgccatcaaggttcagtggcagtggatcaggcacacagtattccctcaagatcagcagcctgcagtctgaagattttgggacttattactgtcaacatttttggggtactccgtggacgttcggtggaggcaccaagctggaaatcaaa

SEQ ID NO: 444 - DNA, 336 bp, Artificial Sequence
  Synthetic: NP3_A7
gacatccagatgactcagtctccagcctctgtacctgtcactcctggagagtcagtatccatctcctgcaggtctagtaagagtctcctgcatagtaatggcaacacttacgtgtattggttcctgcagaggccaggccagtctcctcggctcctgatatatcggatgtccaaccttgcctcaggagtcccagacaggttcagtggcagtgggtcaggaactgctatcacactgagaatcagtagagtggaggctgaggatgtgggtgtttattcctgtatgcaacatctagaatatcctctcacgttcggtgctgggaccaagctggaaataaaa

SEQ ID NO: 445 - DNA, 333 bp, Artificial Sequence
  Synthetic: NP3_A8
gacattgtgatgacccagtctcctgcttccttagttgtatctctggggcagagggccaccatctcatgcagggccagcaaaagtgtcagtgcttctggctatagttatatacactggtaccaacagattccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgctcacgttcggtactggcaccaagctggaaatcaaa

SEQ ID NO: 446 - DNA, 336 bp, Artificial Sequence
  Synthetic: NP3_A9
gaaaatgttctcacccagtctccagcctccaatccagtcactcttggaacatcagcttccatctcctgcaggtctagtaagagtctcctacatagtaatggcttcacttatttgtattggtatctgcagaagccaggccagtctcctcagctcctgatttatcagatgtcctaccttgcctcaggagtcccagacaggttcagtagcagtgggtcaggaactgatttcacactgaggatcagcagagtggaggctgaggatgtgggtgtttattactgtgctcaaaatctagaacttccgttcacgttcggctcgggcaccaagctggaaatcaaa

SEQ ID NO: 447 - DNA, 333 bp, Artificial Sequence
  Synthetic: NP3_A10
gaaaatgttctcacccagtctccagcttctttgactgtctctctaggacagagggccaccctctcctgcaaggccagccaaagtgttgattatgatggtgatagttatatgaactggtaccaacagaaaccaggtcagccacccaaactcctcatctatgctgcatcctatctacaatctgggatcccagccaggtttagtggcagtgggtctgggacagacttcaccctcaacatccatcctgtggaggaggaggatgctgcaacctattactgtcagcaaagtattgaggatccgtacacgttcggagggggcaccaagctggaaatcaaa

SEQ ID NO: 448 - DNA, 321 bp, Artificial Sequence
  Synthetic: NP3_A11
gacatccagatgacccagtctccatcctccctatctgtttctgtgggagagactgtcaccatcacttgtcgagcaagtgagaatatttacagtagtttggcatggtatcagcagaaacagggaaaatctcctcagctcctggtctatactgcaacagacttagcagatggtgtgccgtcaaggttcagtggcagtggatcaggctcgcagtattccctcaggatcaacagcctgcagtctgaagattttgggagttattactgtcaacatttttggggttttccgtggacgttcggtggaggcaccaagctggaaatcaaa

SEQ ID NO: 449 - DNA, 336 bp, Artificial Sequence
  Synthetic: NP3_B1
gatgttgtgatgacccagactccactcactttgtcggttaccattggacaaccagcctccatctcttgcaagtcaagtcagaccctcttagatagtgatggaaagacatatttgaattggttgttccagaggccagggcagtctccaaaacgcctaatctatctggtgtctgaactggactctggagtccctgacaggttcactggcagtggatcagggacagatttcacactgaaaatcagcagagtggaggctgaggatttgggagtttattattgctggcaaggtacacattttcctcagacgttcggtggagggaccaagctggagctgaaa

SEQ ID NO: 450 - DNA, 321 bp, Artificial Sequence
  Synthetic: NP3_B2
gatatccagatgacacagactacatcctccctgtctgcctctctgggagacagagtcaccatcaattgcagtgcaagtcagggcattagcaattatttaaactggtatcagcagaaaccagatggaactgttaaactcctgatctatttcacatcaagtttacactctgggttcccatcgaggttcagtggcagtgggtctgggacagatttttctctcaccatcagcaacctggaacctgaagatattgccacttactattgtcagcaatatagtaagcttccattcacgttcggctcggggacaaagttggaaataaaa

SEQ ID NO: 451 - DNA, 330 bp, Artificial Sequence
  Synthetic: NP3_B3
attgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtacacgttcggaggggggaccaagctggagctcaaa

SEQ ID NO: 452 - DNA, 318 bp, Artificial Sequence
  Synthetic: NP3_B4
gacatccagatgacccagtctccatccatcatgtctgcatctccaggggaaaaggtcaccatgacctgcagtgccagctcaagtgtaagttacatgcactggtaccagcagaagtcaagcacctcccccaaactctggatttatgacacatccgaactggcttctggagtcccaggtcgcttcagtggcagtaggtctggaaactcttactctctcacgatcagcagcatggaggctgaagatgttgccacttattactgttttcaggggagtgggtatccactaacgttcggaggggggaccaagctggagctgaaa

SEQ ID NO: 453 - DNA, 318 bp, Artificial Sequence
  Synthetic: NP3_B5
gaaaatgttctcacccagtctccagcaatcatgtctgcatctccaggggagaaggtcaccatgacctgcagtgccagctcaagtgtaaattacatgcactggtaccagcagaggccaggatcctcgcccaaaccctggatttatgacacatccaacctggcttctggaatccctgtacgcttcagtggcagtggatctgggacctcttactctctcataatcagcagcatggaggctgaagatgctgccacttattactgccatcagcggagtagtttcccattcacattcggctcggggacaaagttggaaatcaaa

SEQ ID NO: 454 - DNA, 321 bp, Artificial Sequence
  Synthetic: NP3_B8
gacatccagatgaaccagtctccatcctccctgtctgcctctctgggaggcagagtcaccatcagttgcagtgcaagtcagggcattagcaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcaacagtataataagcttccgtacacgttcggagggggcaccaagctggaaatcaaa

SEQ ID NO: 455 - DNA, 321 bp, Artificial Sequence
  Synthetic: NP3_B9
gacatccagatgactcagtctccagcctccctatctgtatctgtgggagaaactgtcaccatcacatgtcgagcaagtgagaatatttacagtaatttagcatggtatcagcagaaacagggaaaatctcctcagctcctggtcaatggtgcaacagacttagcagatggtgtgccctcaaggttcagtggcagtggatcaggcacacaatattccctcaagatcaacagcctgcagtctgaagattttgggagttattactgtcaacttttttggggtactccgtggacgttcggtggagggaccaagctggaaatcaaa

SEQ ID NO: 456 - DNA, 333 bp, Artificial Sequence
  Synthetic: NP3_B10
gacattgtgatgacacagtctccatcctctttggctgtatctctagggcagagggccaccatctcctgcaaggccagccaaagtgttgattttgatggtaagagttatatgaactggtaccaacagaaaccaggacagccaccgaaactcctcatctatgttgcatccaatctagaatctgggattcctgccaggtttagtggcagtgggtctgggacagatttcaccctcaacatccatcctgtggaagaagaggatgctgcaacctattactgtcagcaaactaatgaggatccgctcacgttcggtactggcaccaagctggaaatcaaa

SEQ ID NO: 457 - DNA, 333 bp, Artificial Sequence
  Synthetic: NP3_B12
gacattgtgctgacccaatctccagcttctttggctgtgtctctagggcagagggccaccatctcctgcaaggccagccaaagtgttgattatgatggttatagttatatgaactggtaccaacagaaaccaggacaaccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgcggatccgttcacgttcggaggggggaccaagctggagctgaaa

SEQ ID NO: 458 - DNA, 321 bp, Artificial Sequence
  Synthetic: NP3_C3
gacattgtgatgactcagtctccagcctccctatctgtatctgtgggagaaactgtcaccatcacatgtcgagcaagtgaaaatatttacagtaatttagcatggtatcagcagaaacagggaaaatctcctcagctcctggtctatgttgcgacagacttagcagatggtgtgccatcaaggttcagtggcagtggatcaggcacacagtattccctcaagatcaacagcctgcagtctgaagattttgggagttattactgtcaacatttttggggtactccgtggacgttcggtggagggaccaagctggagctgaaa

SEQ ID NO: 459 - DNA, 336 bp, Artificial Sequence
  Synthetic: NP3_C4
gatattgtgatgactcaggctgcacccactttgtcggttaccattggacaaccagcctccatctcttgcaagtcaagtcagagcctcttagatagtgatggaaagacatatttgaattggttgttacagaggccaggccagtctccaaggcgcctaatctatctggtgtctaaactggactctggagtccctgacaggttcactggcagtggatcagggacagacttcacactaaaaatcagcagagtggaggctgaggatttgggagtttattattgctggcaaggtacacattttcctcggacgttcggtggagggaccaagctggaaatcaaa

SEQ ID NO: 460 - DNA, 321 bp, Artificial Sequence
  Synthetic: NP3_C5
gacatccagatgactcagtctccagcctccctatctgtatctgtgggagaaactgtcaccatcacatgtcgagcaagtgagaatatttacagtaatttagcatggtatcagcagaaacagggaaaatctcctcagctcctggtctatggtgcaacagacttagcagatggtgtgccatcaaggttcagtggcagtggatcaggcacacaatattccctcaagatcaacagcctgcagtctgaagattttgggagttattactgtcaacatttttggggtactccgtggacgttcggtggagggaccaagctggagctgaaa

SEQ ID NO: 461 - DNA, 333 bp, Artificial Sequence
  Synthetic: NP3_C6
gacatcaagatgacccagtctccatcctctttggctgtgtctctagggcagagggccaccatctcctgcaaggccagccaaagtgttgattatgatggtgatagttatatgaactggtaccaacagagaccaggacagccacccaaactcctcatctatgctgcatccaatctagaatcggggatcccagccaggtttagtggcagtgggtctggggcagacttcaccctcaacatccatcctgtggaggaggaggatgctgcaacctattactgtcagcaaagtaatgaggatccgtggacgttcggtggagggaccaagctggaaatcaaa

SEQ ID NO: 462 - DNA, 333 bp, Artificial Sequence
  Synthetic: NP3_C8
gacattgtgatgacccagtctccagcctctttggctgtgtctctagggcagagggccaccatctcctgcaaggccagccaaagtgttgattatgatggtgatagttatatgagctggtacctacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtattggggatccgtggacgttcggtggaggcaccaagctggaaatcaaa

SEQ ID NO: 463 - DNA, 333 bp, Artificial Sequence
  Synthetic: NP3_C9
gacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgttgcatccaatctagaatctgggatccctgccaggtttagtggcagtgggtctgggacagacttcaccctcaacatccatcctgtggaggatgaggatgctgcaacctattactgtcagcaaactaatgaggatccgctcacgttcggtgctgggaccaagctggagctgaaa

SEQ ID NO: 464 - DNA, 318 bp, Artificial Sequence
  Synthetic: NP3_C10
gacatcaagatgacccagtctccatcaatcatgtctgcatctccaggggagaaggtcaccatgacctgcagtgccagctcaagtgtaagttacatgtactggttccaacagaagccaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtggttacccgtacacgttcggaggggggaccaagctggaaatcaaa

SEQ ID NO: 465 - DNA, 324 bp, Artificial Sequence
  Synthetic: NP3_C11
gacattgtgatgacacagtctccatccctcatgtctacatctctaggggaacgggtcaccatgacctgcactgccaactcaagtgtaagttccaattacatgcactggtaccagcagaagccaggatcctcccccaaactctggatttatagcacatccaacctggcttctggagtcccagctcgcttcagtggcagtgggtctgggacctcttactctctcacaatcagcaacatggaggctgaagatgctgccacttattactgccaccagtttcatcgttccccacggacgttcggtggagggacaaagttggaaatcaaa

SEQ ID NO: 466 - DNA, 336 bp, Artificial Sequence
  Synthetic: NP3_D1
gatattgtgatgactcaggctgcacccactttgtcggttacccttggacaaccagcctccatctcttgcaagtcaagtcagagcctcttagatagtgatggaaagacatatttgagttggttgttacagaggccaggccagtctccaaagcgcctaatctatctggtgtctgaactggactctggagtccctgacaggttcactggcagtggatcagggacagatttcacactgaaaatcagcagagtggaggctgaggatttgggagtttattattgctggcaaggtacacattttcctcagacgttcggtggagggaccaagctggaaatcaaa

SEQ ID NO: 467 - DNA, 333 bp, Artificial Sequence
  Synthetic: NP3_D4
gaaaatgttctcacccagtctccagcttctttgactgtctctctagggcagagggccaccctctcctgcaaggccagccaaagtgttgattatgatggtgatagttatatgaactggtaccaacagaaaccaggacagccacccaaactcctcatctatgctgcatcctatccagaatctgggatcccagccaggtttagtggcagtgggtctgggacagacttcaccctcaacatccatcctgtggaggaggaggatgctgcaacctattactgtcagcaaagtattgaggatccgtacacgttcggagggggcaccaagctggaaatcaaa

SEQ ID NO: 468 - DNA, 321 bp, Artificial Sequence
  Synthetic: NP3_D5
gacattgtgatgacccagtctcacaaattcatgtccacatcagtaggagacagggtcagcatcacctgcaaggccagtcaggatgtgggtactgctgtagcctggtatcaacagaaaccaggacaatctcctaaactactgatttactgggcatccacccggcacactggagtccctgatcgcttcacaggcagtggatctgggacagatttcactctcaccattagcaatgtgcagtctgaagacttggcagattatttctgtcaccaatatagcagctatccgtacacgttcggaggggggaccaagctggaaatcaaa

SEQ ID NO: 469 - DNA, 321 bp, Artificial Sequence
  Synthetic: NP3_E2
gacattgtgatgacccagtctcacaaattcatgtccacatcagtaggagacagggtcagcatcacctgcaaggccaatcaggatgtgggttctgctgtagcctggtatcaggagaaaccagggcaatctcctaaacttctgatttactggacatccacccgacacactggagtccctgatcgcttcacaggcaatggatctgggacagatttcactctcaccattagcaatgtccagtctgaagacctggtagattatttctgtcagcaatatagcaggtatcctctcacgttcggctcggggaccaagctggagctgaaa

SEQ ID NO: 470 - DNA, 321 bp, Artificial Sequence
  Synthetic: NP3_E3
gacattgtgctgacccaatctccagcctccctatctgtatctgtgggagaaactgtcaccatcacatgtcgagcaagtgagaatatttacagtaatttagcatggtatcagcagaaacagggacaatctcctcagctcctagtctatgctgcaacagacttagcagatggtgtgccatcaaggttcagtggcagtggatcaggcacacagtattccctcaagatcaacagcctgcagtctgaagattttgggaattattactgtcaacatttttggggtactccgtggacgttcggtggaggcaccaagctggaaatcaaa

SEQ ID NO: 471 - DNA, 321 bp, Artificial Sequence
  Synthetic: NP3_E4
gacatcaagatgacccagtctccatcctccctgtctgcctctctgggagacagagtcaccatcagttgcagtgcaagtcaggacattagcgattatttaaactggtatcagcagaaaccagatggaactgttaaactcctgatctattacacatcaagtttacactcaggggtcccatcaaggttcagtggcagtgggtctgggacagattattctctcaccatcagcaacctggaacctgaagatattgccacttactattgtcagcagtatagtaagcttccatacacgttcggagggggcaccaagctggaaatcaaa

SEQ ID NO: 472 - DNA, 321 bp, Artificial Sequence
  Synthetic: NP3_E5
gatatccagatgacacagactacatcctccctgtctgcctctctgggagacagagtcaccatcaattgcagtgcaagtcagggcattagcaattatttaaactggtatcagcagaaaccagatggaactgttaaactcctgatctattacacatcaattttacactcaggagtcccatcaaggttcagtggcagtgggtctgggacagattattctctcaccatcagcaacctggaacctgaagatattgccacttactattgtcagcagtatagtaagcttccattcacgttcggctcggggacaaagttggaaatcaaa

SEQ ID NO: 473 - DNA, 330 bp, Artificial Sequence
  Synthetic: NP3_E6
attgtgctgacccaatctccagcttctttggctttgtctctagggcagagggccaccatctcctgcaaggccagccaaagtgttgattatgatggtgatagttatatgaactggtaccaacagaaaccaggacagccacccaaactcctcatctctgctgcatccaatctagaatctgggatcccagccaggtttggtggcagtgggtctgggacagacttcaccctcaacatccatcctgtggaggaggaggatgctgcaacctattactgtcagcaaagtaatgaggatccgttcacgttcggagggggcaccaagctggaaatcaaa

SEQ ID NO: 474 - DNA, 321 bp, Artificial Sequence
  Synthetic: NP3_E11
gacattgtgatgacccagtctcacaaattcatgtccacatcagtaggagacagggtcagcatcacctgcaaggccagtcaggatgtgggttctgctgtagcctggtatcaacagaaaccagggcaatctcctaaactactgatttactggacatccacccggcacactggagtccctgatcacttcacaggcagtggatctgggacagatttcactctcaccattaacaatgtgcagtctgaagacttggcagattatttctgtcaccaatatagcagctatccgtacacgttcggaggggggaccaagctggaaatcaaa

SEQ ID NO: 475 - DNA, 321 bp, Artificial Sequence
  Synthetic: NP3_F4
gacatcaagatgacccagtctccatcctccctatctgtatctgtgggagaaactgtcaccatcacatgtcgagcaagtgaaaatatctacagtaatttagcgtggtatcagcagaaacagggaaaatctcctcagctcctggtctctgctgcaacaaacttagcagataatgtgccatcaaggttcagtggcagtggatcaggcacacagtattccctcaagatcaatagcctgcagtctgaagactttgggagttattactgtcaacatttttggggttttcctcggacgttcggtggaggcaccaagctggaaatcaaa

SEQ ID NO: 476 - DNA, 333 bp, Artificial Sequence
  Synthetic: NP3_F7
gacattgtgctgacccaatctccaggttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagtcaggtttagtggcagtgggtctgggacagacttcaccctcaatatccatcctgtggaggaggaggatgctgcaacctattactgtcagcaaagtaatgaggatccgtggacgttcggtggcgggaccaagctggagctgaaa

SEQ ID NO: 477 - DNA, 324 bp, Artificial Sequence
  Synthetic: NP3_F8
gacatccagatgacccagtctccatccataatggctgcctctgtggggcagaaggtcaccatgacctgcagtgccagctcaagtgttagttccagttacttgcactggtaccagcagaagtcaggcgcttcccccaaacccttgattcataggacatccaacctggcttctggagtcccacctcgcttcagtggcagtgggtctgggacctcttactctctcacaatcagcagcgtggaggctgaagatgatgcaacttattactgccagcagtggagtggttatccattcacgttcggtactgggacaaagttggaaatcaaa

SEQ ID NO: 478 - DNA, 321 bp, Artificial Sequence
  Synthetic: NP3_F10
gatatccagatgacacagactacatcctccctgtctgcctctctgggagacagagtcaccatcagttgcagtgcaagtcaggacattagcgattatttaaactggtatcaacagaaaccagatggaactattaagctcctgatctattacacatcaagtttacactcaggagccccatcaaggttcagtggcagtgggtctgggacagattattctctcaccatcagcaacctggaacctgaagatattgccacttactattgtcaacagtatagtaagcttccgtggacgttcggtggagggaccaagctggagctgaaa

SEQ ID NO: 479 - DNA, 330 bp, Artificial Sequence
  Synthetic: NP3_G1
attgtgctgacacagtctcctgcttccttagctgtttctctggggcagagggccaccatctcatgcagggccagcaaaagtgtcagtgcatctggctatagttatctccactggtaccaacagaaaccaggacagccacccaaactcctcatctatcttgcatccaacctaccatctggggtccctgccaggttcagtggcagtgggtctgggacagacttcaccctcaacatccatcctgtggaggaggaggatgctgcaacctattactgtcagcaccgtcgggagattccgctcacgttcggtgctggcaccaagctggaaatcaaa

SEQ ID NO: 480 - DNA, 321 bp, Artificial Sequence
  Synthetic: NP3_G5
gacattgtgatgacccagtctcacaaattcatgtccacatcagtaggagacaggatcagcatcacctgcaaggccagtcaggatgtgagtactgctgtagtctggtatcaacagaaaccaggacagtctcttaaactactgatttactcggcatcctaccggtacactggagtccctgatcgcttcactggcagtggatctgggacggatttcactttcaccatcagcagtgtgcaggctgaagacctggcaatttattactgtcagcaatattatagtactccattcacgttcggctcggggacaaagttggaaatcaaa

SEQ ID NO: 481 - DNA, 321 bp, Artificial Sequence
  Synthetic: NP3_G6
gacatccagatgactcagtctccagcctccctgtctgcctctctgggagacagagtcaccatcaattgcagtgcaagtcagggcattagcaattatttaaactggtatcagcagaaaccagatggaactgttaaactcctgatctatttcacatcaagtttacactctgggttcccatcaaggttcagtggcagtgggtctgggacagatttttctctcaccatcagcaacctggaacctgaagatattgccacttactattgtcagcaatatagtaagcttccattcacgttcggctcgggcaccaagctggaaatcaaa

SEQ ID NO: 482 - DNA, 321 bp, Artificial Sequence
  Synthetic: NP3_G7
gacatccagatgactcagtctccagcctccctatctgtatctgtgggagaaactgtcaccatcacatgtcgagcaagtgaaaatatttacagtaatttagcatggtatcagcagaaacagggaaaatctcctcagctcctggtctatgttgcgacagacttagcagatggtgtgccatcaaggttcagtggcagtggatcagacacacagtattccctcaagatcaacagcctgcagtctgaagattttgggagttattactgtcaacatttttggggtactccgtggacgttcggtggaggcaccaagctggaaatcaaa

SEQ ID NO: 483 - DNA, 333 bp, Artificial Sequence
  Synthetic: NP3_G8
gaaaatgttctcacccagtctccagcttctttggctgtgtctctagggcagagggccaccatctcctgcaaggccagccaaagtgttgattatgatggtggtagttatatgaactggtaccaacagaaaccaggacagccacccaaactcctcatctatgctgcatccactctagaatctggaatcccagccgggtttagtggcagtgggtctgggacagacttcaccctcaacatccatcctgtggaggaggaggatgctgcaacctattactgtcagcaaagtaatgaagatccgtggacgttcggtggaggcaccaagctggaaatcaaa

SEQ ID NO: 484 - DNA, 333 bp, Artificial Sequence
  Synthetic: NP3_G9
gacatccagatgacccagtctccatcctctttggctgtgtctctagggcagagggccagcatctcctgcaaggccagccaaagtgttgattatgatggtgagagttatatgaactggtaccaacagaaaccaggacagccacccaaagtcctcatctatgctgcatccaatctagaatctgggatcccagccaggtttagtggcagtggggctgggacagacttcaccctcagcatccatcctgtggaggaggaggatgctgcaacctattactgtcagcaaagttatgaggatccgtggacgttcggtggcgggaccaagctggagctgaaa

SEQ ID NO: 485 - DNA, 333 bp, Artificial Sequence
  Synthetic: NP3_G12
gacattgtgatgacccagtctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttggtggcggtgggtctgggacagacttcaccctcaacatccatcctgtggaggaggaggatgctgcaacctattactgtcagcaaagtaatgaggatccgttcacgttcggaggggggaccaagctggagctgaaa

SEQ ID NO: 486 - DNA, 334 bp, Artificial Sequence
  Synthetic: NP3_H2
gaaaatgttctcacccagtctccagcttctttggctgtgtctctagggcagagggccaccatctcctgcaaggccagtcaaagtgttgattatgatggtgatagttatatgaattggtaccaacagaaaccaggacagccacccaaactcctcatctatgctgcatccagtctagattctgggatcccagccaggtttggtggcagdtgggtctgggacagacttcaccctcaacatccatcctgtggaggaggaggatgctgcaacctattactgtcagcaaagtaatgaggatccgtggacgttcggtggaggcaccaagctggaaatcaaa

SEQ ID NO: 487 - DNA, 321 bp, Artificial Sequence
  Synthetic: NP3_H9
gatgtccagataacccagtctccatcctccctatctgtatctgtgggagaaactgtcaccatcacatgtcgagcaagtgagaatatttacagtaatttagcatggtatcagcagaaacagggaaaatctcctcagctcctggtctatgctgcaacagacttagcagatggtgtgccatcaaggttcagtggcagtggatcaggcacacattattccctcaagatcaacagcctgcagtctgaagattttgggagtttttactgtcaacatttttggggtactccgtggacgttcggtggagggaccaagctggaaatcaaa

SEQ ID NO: 488 - DNA, 321 bp, Artificial Sequence
  Synthetic: NP3_H12
gatattctgatgactcaggctgcaccctccctatctgtatctgtgggagaaactgttaccatcacatgtcgagcaagtgagaatatttacagtgatttagcatggtatcagcataaagagggaaaatctcctcagctcctggtctatgctgcaacagacttagcagatggtgtgccatcaaggttcagtggcagtggatcaggcacacagtattccctcaagatcaacagcctgcagtctgaagattttgggaattattactgtcaacatttttggggtactccgtggacgttcggtggagggaccaagctggaaataaaa

(19) United States
(12) Patent Application Publication
(10) Pub. No.: US 2025/0320278 A1
(43) Pub. Date: Oct. 16, 2025

(54) CHIMERIC ANTI-SARS-COV2 NUCLEOPROTEIN ANTIBODIES

(71) Applicant: LA JOLLA INSTITUTE FOR IMMUNOLOGY, La Jolla, CA (US)

(72) Inventors: Erica Ollmann SAPHIRE, La Jolla, CA (US); Kathryn HASTIE, La Jolla, CA (US); Sara LANDERAS-BUENO, La Jolla, CA (US); Stephanie HARKINS, La Jolla, CA (US)

(21) Appl. No.: 18/570,976

(22) PCT Filed: Apr. 14, 2022
(86) PCT No.: PCT/US2022/024908

§ 371 Date: Dec. 15, 2023

Related U.S. Application Data
(60) Provisional application No. 63/215,297, filed on Jun. 25, 2021.

(51) Int. Cl. C07K 16/10; G01N 33/569

(52) U.S. Cl. CPC C07K 16/1003; G01N 33/56983; C07K 2317/565; C07K 2317/92; G01N 2333/165

(57) ABSTRACT

Compositions and methods are disclosed for diagnosing, treating, and/or preventing coronavirus infection and related pathologies. The compositions include antibodies having certain defined heavy and light chain sequences that recognize the coronavirus nucleoprotein or mutants thereof. In embodiments, the coronavirus is SARS-CoV-2

### CROSS-REFERENCE TO RELATED APPLICATION

This application claims priority to and the benefit of PCT Application No. PCT/US2022/024908, filed Apr. 14, 2022, entitled “CHIMERIC ANTI-SARS-COV2 NUCLEOPROTEIN ANTIBODIES”, which claims priority to, and the benefit of, U.S. Provisional Patent Application Ser. No. 63/215,297, filed Jun. 25, 2021, entitled “CHIMERIC ANTI-SARS-COV2 NUCLEOPROTEIN ANTIBODIES” the entire disclosure of which is incorporated herein by reference in its entirety.

### TECHNICAL FIELD

The present application relates to antibodies, and particularly to antibodies that recognize SARS-CoV-2 nucleoprotein.

### STATEMENT OF FEDERALLY FUNDED RESEARCH

Not applicable.

### INCORPORATION-BY-REFERENCE OF MATERIALS FILED ON COMPACT DISC

This application includes an electronically submitted sequence listing in .txt format. The .txt file contains a sequence listing entitled “LJII1037US_ST25.txt” created on Dec. 3, 2024 and is 214,935 bytes in size. The sequence listing contained in this .txt file is part of the specification and is hereby incorporated by reference herein in its entirety.

### BACKGROUND

Effective countermeasures against the emergence and expansion of the 2019-Novel Coronavirus (SARS-CoV-2) require the development of new tools for detection, diagnosis, monitoring and treatment.

The worldwide spread of SARS-CoV-2 in the human population has resulted in the ongoing COVID-19 pandemic and has already caused more than 491 million infections and more than 6.2 million deaths resulting from SARS-CoV-2 or related diseases and conditions. As such, the aforementioned tools are urgently required.

### SUMMARY

This Summary is provided to introduce a selection of concepts in a simplified form that are further described below in the Detailed Description. This Summary is not intended to identify key aspects or essential aspects of the claimed subject matter.

As embodied and broadly described herein, an aspect of the present disclosure relates to an antibody composition that recognizes a coronavirus nucleoprotein or mutants thereof. In an 25 aspect, the antibody composition comprises a heavy chain having at least 80%, or at least 85%, or at least 90%, or at least 95% sequence identity with any one of SEQ ID NOs: 1-48. In embodiments, the heavy chain comprises any one of SEQ ID NOs: 1-48.

In another aspect, an antibody composition is disclosed comprising a light chain having at least 80%, or at least 85%, or at least 90%, or at least 95% sequence identity with any one of SEQ ID NOs: 49-96. In embodiments, the light chain comprises any one of SEQ ID NOs: 49-96.

In another aspect, an antibody composition is disclosed comprising a heavy chain having at least 80%, or at least 85%, or at least 90%, or at least 95% sequence identity with any one of SEQ ID NOs: 1-48; and a light chain having at least 80%, or at least 85%, or at least 90%, or at least 95% sequence identity with any one of SEQ ID NOs: 48-96. In embodiments, the heavy chain comprises any one of SEQ ID NOs: 1-48; and the light chain comprises any one of SEQ ID NOs: 49-96.

In another aspect, a method of treating an individual infected with a coronavirus is disclosed. The method involves administering to the individual an effective amount of an antibody composition as detailed herein.

In another aspect, a method of preventing a coronavirus infection in an individual is disclosed. The method involves administering to the individual an effective amount of an antibody composition as detailed herein.

In another aspect, a method of identifying a coronavirus in an individual is disclosed. The method involves isolating a biological sample from the individual; incubating the biological sample with an antibody composition as detailed herein; and detecting a biological interaction between the biological sample and the antibody composition.

In another aspect, a method of detecting a coronavirus in a sample is disclosed. The method involves isolating a biological sample; incubating the biological sample with an antibody composition as detailed herein; and detecting a biological interaction between the biological sample and the antibody composition.

In another aspect, a kit for identifying a coronavirus in a biological sample is disclosed. The kit includes an antibody composition as detailed herein; and instructions of use.

In certain aspects, the coronavirus is SARS, MERS, 229E (alpha), NL63 (alpha), OC43 (beta), HKU1 (beta), SARS-CoV-2, or an emerging variant thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), Delta (B.1.617.2 and AY lineages), and Omicron (B.1.1.529) or a variant (including but not limited to BA.1, BA.2, or BA.3) thereof.

In another aspect, an enzyme-linked immunosorbent assay (ELISA) test kit is disclosed. The ELISA test kit includes an antibody composition as detailed herein; and instructions of use.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE FIGURES

In the present application:

FIGS. 1a-1d demonstrate certain ELISA binding data as detailed herein.

### DETAILED DESCRIPTION

A detailed description of one or more embodiments of the invention is provided below along with accompanying figures that illustrate the principles of the invention. The invention is described in connection with such embodiments, but the invention is not limited to any embodiment. The scope of the invention is limited only by the claims. Numerous specific details are set forth in the following description in order to provide a thorough understanding of the invention. These details are provided for the purpose of non-limiting examples and the invention may be practiced according to the claims without some or all of these specific details. For the purpose of clarity, technical material that is known in the technical fields related to the invention has not been described in detail so that the invention is not unnecessarily obscured.

### Overview of Disclosure

The present disclosure describes antibodies that recognize SARS-CoV-2 nucleoprotein. The disclosure also describes certain methods of treatment, both for therapeutic and prophylactic purposes, as well as diagnostics capable of detecting the presence of SARS-CoV-2 and methods of diagnosing.

### Definitions and Interpretation

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

If and as used herein, the term “administering”, when used in relation to an expression vector, nucleic acid molecule, or a delivery vehicle (such as a chitosan nanoparticle) to a cell, refers to transducing, transfecting, electroporation, translocating, fusing, phagocytosing, shooting or ballistic methods, etc., i.e., any means by which a protein or nucleic acid can be transported across a cell membrane and preferably into the nucleus of a cell.

Unless otherwise indicated, a particular amino acid sequence that is recited herein also implicitly encompasses conservatively modified variants. As a non-limiting example, any of the disclosed amino acid sequences for portions of an anti-SARS-CoV-2 nucleoprotein antibody implicitly encompass conservatively modified variants.

If and as used herein, the terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins. The polypeptide sequences are displayed herein in the conventional N-terminal to C-terminal orientation.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, carboxyglutamate, and O-phosphoserine. The expression “amino acid analogs” refers to compounds that have the same basic chemical structure as a naturally occurring amino acid, i.e., an alpha. carbon that is bound to a hydrogen, a carboxyl group, an amino group, and an R group, e.g., homoserine, norleucine, methionine sulfoxide, methionine, and methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

As used herein, the term “CDR” refers to a complementary determine region of an antibody. A CDR is part of a variable chain of an antibody that binds to an antigen.

As used herein, the phrase “consensus sequence” refers to a sequence that results from a comparison of at least two sequences which contains all possible amino acid residues at each position of the at least two sequences.

If and as used herein, the term “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon in an amino acid herein, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid and nucleic acid sequences, individual substitutions, deletions or additions that alter, add or delete a single amino acid or nucleotide or a small percentage of amino acids or nucleotides in the sequence create a “conservatively modified variant,” where the alteration results in the substitution of an amino acid with a chemically similar amino acid. Conservative substitution tables providing functionally similar amino acids are well known in the art.

For example, the following groups each contain amino acids that are conservative substitutions for one another (see, e.g., Creighton, Proteins (1984) W.H. Freeman, New York, pages 6-20, for a discussion of amino acid properties):

Alanine (A), Glycine (G)
Serine (S), Threonine (T)
Aspartic acid (D), Glutamic acid (E)
Asparagine (N), Glutamine (Q)
Cysteine (C), Methionine (M)
Arginine (E), Lysine (K), Histidine (H)
Isoleucine (I), Leucine (L), Valine (V)
Phenylalanine (F), Tyrosine (Y), Tryptophan
(W)

In light of the present disclosure, in particular in view of the experimental data described in the examples of the present text, the person of skill will readily understand which amino acid may be substituted, deleted or added to a given sequence to create a conservatively modified variant comprising an amino acid sequence which is at least at least 95%, or at least 96%, or at least 97%, or at least 98%, or at least 99%, identical to one or more amino acid sequence set forth in the table above without undue effort.

If and used herein, the terms “treating” or “treatment” refers to a process by which an infection, such as infection with a coronavirus, or a disease or the symptoms of an infection or a disease associated with a viral strain are prevented, alleviated or completely eliminated. As used herein, the term “prevented” or “preventing” refers to a process by which an infection or a disease or symptoms of an infection or a disease associated with a virus, such as a coronavirus, are averted prior to infection. In certain aspects, the coronavirus is SARS, MERS, 229E (alpha), NL63 (alpha), OC43 (beta), HKU1 (beta), SARS-CoV-2, or an emerging variant thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), Delta (B.1.617.2 and AY lineages), and Omicron (B.1.1.529) or a variant (including but not limited to BA.1, BA.2, or BA.3) thereof.

If and as used herein, a “sufficient amount” or “effective amount” or an “amount sufficient” or an “amount effective” refers to an amount that provides, in single (e.g., primary) or multiple (e.g., booster) doses, alone or in combination with one or more other compounds, treatments, therapeutic regimens or agents (e.g., a drug), a long term or a short term detectable or measurable improvement in a given subject or any objective or subjective benefit to a given subject of any degree or for any time period or duration (e.g., for minutes, hours, days, months, years, or cured).

An amount sufficient or an amount effective need not be therapeutically or prophylactically effective in each and every subject treated, nor a majority of subjects treated in a given group or population. An amount sufficient or an amount effective means sufficiency or effectiveness in a particular subject, not a group of subjects or the general population. As is typical for such methods, different subjects will exhibit varied responses to treatment.

If and used herein, the expression “an acceptable carrier” refers to a vehicle for containing a compound that can be administered to a subject without significant adverse effects.

As used herein, the term “adjuvant” means a substance added to the composition of the invention to increase the composition's immunogenicity. The mechanism of how an adjuvant operates is not entirely known. Some adjuvants are believed to enhance the immune response (humoral and/or cellular response) by slowly releasing the antigen, while other adjuvants are strongly immunogenic in their own right and are believed to function synergistically.

With respect to the present disclosure, an adjuvant may be selected from aluminum hydroxide or mineral oil, and a stimulator of immune responses, such as Bordetella pertussis or Mycobacterium tuberculosis derived proteins. Suitable adjuvants are commercially available as, for example, Freund's Incomplete Adjuvant and Complete Adjuvant (Pifco Laboratories, Detroit, Mich.); Merck Adjuvant 65 (Merck and Company, Inc., Rahway, N.J.); aluminum salts such as aluminum hydroxide gel (alum) or aluminum phosphate; salts of calcium, iron or zinc; an insoluble suspension of acylated tyrosine acylated sugars; cationically or anionically derivatized polysaccharides; polyphosphazenes; biodegradable microspheres; and Quil A. Suitable adjuvants also include, but are not limited to, toll-like receptor (TLR) agonists, particularly toll-like receptor type 4 (TLR-4) agonists (e.g., monophosphoryl lipid A (MPL), synthetic lipid A, lipid A mimetics or analogs), aluminum salts, cytokines, saponins, muramyl dipeptide (MDP) derivatives, CpG oligos, lipopolysaccharide (LPS) of gram-negative bacteria, polyphosphazenes, emulsions, virosomes, cochleates, poly(lactide-co-glycolides) (PLG) microparticles, poloxamer particles, microparticles, liposomes, oil-in-water emulsions, MF59, and squalene. In some embodiments, the adjuvants are not bacterially-derived exotoxins. In an embodiment, adjuvants may include adjuvants which stimulate a Th1 type response such as 3DMPL or QS21. Adjuvants may also include certain synthetic polymers such as poly amino acids and co-polymers of amino acids, saponin, paraffin oil, and muramyl dipeptide. Adjuvants also encompass genetic adjuvants such as immunomodulatory molecules encoded in a co-inoculated DNA, or as CpG oligonucleotides. The co-inoculated DNA can be in the same plasmid construct as the plasmid immunogen or in a separate DNA vector. The reader can refer to Vaccines (Basel). 2015 June; 3(2): 320-343 for further examples of suitable adjuvants.

If and used herein, the terms “determining,” “measuring,” “evaluating,” “assessing,” and “assaying” generally refer to any form of measurement, and include determining if an element is present or not in a biological sample. These terms include both quantitative and/or qualitative determinations, which both require sample processing and transformation steps of the biological sample. Assessing may be relative or absolute. The phrase “assessing the presence of” can include determining the amount of something present, as well as determining whether it is present or absent.

If and used herein, the expression “biological sample” includes, in the present disclosure, any biological sample that is suspected of comprising a T cell, such as for example but without being limited thereto, blood and fractions thereof, urine, excreta, semen, seminal fluid, seminal plasma, prostatic fluid, pre-ejaculatory fluid (Cowper's fluid), pleural effusion, tears, saliva, sputum, sweat, biopsy, ascites, amniotic fluid, lymph, vaginal secretions, endometrial secretions, gastrointestinal secretions, bronchial secretions, breast secretions, and the like. In one non-limiting embodiment, a herein described biological sample can be obtained by any known technique, for example by drawing, by non-invasive techniques, or from sample collections or banks, etc.

If and used herein, the expression “treatment” includes inducing, enhancing, or sustaining an immune response against a viral infection or symptoms associated thereto. For example, the treatment may induce, increase, promote or stimulate anti-viral virus activity of immune system cells in a subject following the treatment. In non-limiting examples, the immune system cells may include adaptive immune cells, such as T cells, including CD4+ T cells, CD8+ T cells, and/or B cells, or innate immune cells, such as macrophages and/or neutrophils.

If and used herein, the expression “therapeutically effective amount” may include the amount necessary to allow the component or composition to which it refers to perform its immunological role without causing overly negative effects in the host to which the component or composition is administered. The exact amount of the components to be used or the composition to be administered will vary according to factors such as the type of condition being treated, the type and age of the subject to be treated, the mode of administration, as well as the other ingredients in the composition.

As used herein, the term “virus” generally refers to a coronavirus or any mutant form thereof. As used herein, the term “SARS-CoV-2” refers to all phylogenetic samples of SARS-CoV-2 genomes, as well as any and all mutant strains thereof. As used herein, the term “nucleoprotein” refers to a protein expressed by a coronavirus, and includes, without limitation, reference to SARS-CoV-2 Nucleocapsid Protein (Severe acute respiratory syndrome coronavirus 2 nucleocapsid, 2019 novel coronavirus nucleoprotein, SARS-CoV-2 NP, SARS-CoV-2 N protein, COVID-19).

Other examples of implementations will become apparent to the person skilled in the art in view of the teachings of the present description and as such, will not be further described here.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

Any and all references cited throughout the specification are hereby incorporated by reference in their entirety for all purposes.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

### Detailed Description of Aspects and Embodiments of the Disclosure

As embodied and broadly described herein, an aspect of the present disclosure relates to an antibody composition comprising a heavy chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 1-48. In embodiments, the heavy chain comprises any one of SEQ ID NOs: 1-48.

In an aspect, a nucleotide sequence that encodes for a heavy chain sequence of an antibody composition is disclosed having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 393-340. In embodiments, the nucleotide sequence that encodes for the heavy chain sequence of the antibody composition comprises any one of SEQ ID NOs: 393-440.

In another aspect, an antibody composition is disclosed comprising a light chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 48-96. In embodiments, the light chain comprises any one of SEQ ID NOs: 48-96.

In an aspect, a nucleotide sequence that encodes for a light chain sequence of an antibody composition is disclosed having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 441-488. In embodiments, the nucleotide sequence that encodes for the light chain sequence of the antibody composition comprises any one of SEQ ID NOs: 441-488.

In another aspect, an antibody composition is disclosed comprising a heavy chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 1-48; and a light chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 49-96. In embodiments, the heavy chain comprises any one of SEQ ID NOs: 1-48; and the light chain comprises any one of SEQ ID NOs: 49-96.

In another aspect, an antibody composition is disclosed comprising a consensus sequence of a heavy chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 385. In embodiments, the consensus sequence of the heavy chain comprises SEQ ID NO: 385.

In another aspect, an antibody composition is disclosed comprising a consensus sequence of a light chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 386. In embodiments, the consensus sequence of the light chain comprises SEQ ID NO: 386.

In another aspect, an antibody composition is disclosed comprising a consensus sequence of a heavy chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 385; and a consensus sequence of light chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 386. In embodiments, the consensus sequence of the heavy chain comprises SEQ ID NO: 385; and the consensus sequence of the light chain comprises SEQ ID NO: 386.

In another aspect, an antibody composition is disclosed comprising a heavy chain CDR1 (CDRH1) sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 97-144. In embodiments, the CDRH1 sequence comprises any one of SEQ ID NOs: 97-144.

In another aspect, an antibody composition is disclosed comprising a heavy chain CDR2 (CDRH2) sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 145-192. In embodiments, the CDRH2 sequence comprises any one of SEQ ID NOs: 145-192.

In another aspect, an antibody composition is disclosed comprising a heavy chain CDR3 (CDRH3) sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 193-240. In embodiments, the CDRH3 sequence comprises any one of SEQ ID NOs: 193-240.

In another aspect, an antibody composition is disclosed comprising a light chain CDR1 (CDRL1) sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 241-288. In embodiments, the CDRL1 sequence comprises any one of SEQ ID NOs: 241-288.

In another aspect, an antibody composition is disclosed comprising a light chain CDR2 (CDRL2) sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 289-336. In embodiments, the CDRL2 sequence comprises any one of SEQ ID NOs: 289-336.

In another aspect, an antibody composition is disclosed comprising a light chain CDR3 (CDRL3) sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 337-384. In embodiments, the CDRL3 sequence comprises any one of SEQ ID NOs: 337-384.

In another aspect, an antibody composition is disclosed comprising one or more CDR sequences of a heavy chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one or more of SEQ ID NOs: 97-240; and one or more CDR sequences of a light chain having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one of SEQ ID NOs: 241-384. In embodiments, the one or more CDR sequences of the heavy chain comprises any one or more of SEQ ID NOs: 97-240; and the one or more CDR sequences of the light chain comprises any one or more of SEQ ID NOs: 241-384.

In another aspect, an antibody composition is disclosed comprising a consensus sequence of a CDRH1 sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 387. In embodiments, the consensus sequence of the CDRH1 sequence comprises SEQ ID NO: 387.

In another aspect, an antibody composition is disclosed comprising a consensus sequence of a CDRH2 sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 388. In embodiments, the consensus sequence of the CDRH2 sequence comprises SEQ ID NO: 388.

In another aspect, an antibody composition is disclosed comprising a consensus sequence of a CDRH3 sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 389. In embodiments, the consensus sequence of the CDRH3 sequence comprises SEQ ID NO: 389.

In another aspect, an antibody composition is disclosed comprising a consensus sequence of a CDRL1 sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 390. In embodiments, the consensus sequence of the CDRL1 sequence comprises SEQ ID NO: 390.

In another aspect, an antibody composition is disclosed comprising a consensus sequence of a CDRL2 sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 391. In embodiments, the consensus sequence of the CDRL2 sequence comprises SEQ ID NO: 391.

In another aspect, an antibody composition is disclosed comprising a consensus sequence of a CDRL3 sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with SEQ ID NO: 392. In embodiments, the consensus sequence of the light chain CDRL3 sequence comprises SEQ ID NO: 392.

In another aspect, an antibody composition is disclosed comprising one or more of a consensus sequence of a heavy chain CDR sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one or more of SEQ ID NOs: 387, 388, and 389; and one or more of a consensus sequence of a light chain CDR sequence having at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one or more of SEQ ID NOs: 390, 391, and 392. In embodiments the one or more consensus heavy chain CDR sequence comprises any one or more of SEQ ID NOs: 387, 388, and 389; and the one or more consensus light chain CDR sequence comprises any one or more of SEQ ID NOs: 390, 391, and 392.

The above-mentioned antibody compositions also include any and all variants, modifications, homologues, derivatives or subsequences thereof.

In another aspect, a method of treating an individual infected with a coronavirus is disclosed. The method involves administering to the individual an effective amount of an antibody composition as detailed herein. In certain aspects, the coronavirus is SARS-CoV-2.

In another aspect, a method of preventing a coronavirus infection in an individual is disclosed. The method involves administering to the individual an effective amount of an antibody composition as detailed herein.

In another aspect, a method of identifying coronavirus in an individual is disclosed. The method involves isolating a biological sample from the individual; incubating the biological sample with an antibody composition as detailed herein; and detecting a biological interaction between the biological sample and the antibody composition.

In another aspect, a kit for identifying coronavirus in a biological sample is disclosed. The kit includes an antibody composition as detailed herein; and instructions of use.

In another aspect, an enzyme-linked immunosorbent assay (ELISA) test kit is disclosed. The ELISA test kit includes an antibody composition as detailed herein; and instructions of use.

In accordance with the invention, treatment methods are provided that include therapeutic (following infection with a coronavirus) and prophylactic (prior to coronavirus exposure, infection or pathology) methods. For example, therapeutic and prophylactic methods of treating a subject for a viral infection include treatment of a subject having or at risk of having a viral infection or pathology, treating a subject with a viral infection, and methods of protecting a subject from a viral infection, to decrease or reduce the probability of a viral infection in a subject, to decrease or reduce susceptibility of a subject to a viral infection, or to inhibit or prevent a viral infection in a subject, and to decrease, reduce, inhibit or suppress transmission of a virus from a host to a subject.

Such methods include the administration of the antibodies disclosed herein, or nucleic acids encoding the antibodies disclosed herein, to therapeutically or prophylactically treat a subject having or at risk of having a virus infection or pathology. Accordingly, methods can treat the virus infection or pathology, or provide the subject with protection from infection (e.g., prophylactic protection).

In certain aspects, the coronavirus is SARS, MERS, 229E (alpha), NL63 (alpha), OC43 (beta), HKU1 (beta), SARS-CoV-2, or an emerging variant thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), Delta (B.1.617.2 and AY lineages), and Omicron (B.1.1.529) or a variant (including but not limited to BA.1, BA.2, or BA.3) thereof.

In particular embodiments, one or more disorders, diseases, physiological conditions, pathologies and symptoms associated with or caused by a coronavirus (e.g., SARS-CoV-2) viral infection or pathology will respond to treatment. In particular methods embodiments, treatment methods reduce, decrease, suppress, limit, control or inhibit viral numbers or titer; reduce, decrease, suppress, limit, control or inhibit pathogen proliferation or replication; reduce, decrease, suppress, limit, control or inhibit the amount of a pathogen protein; or reduce, decrease, suppress, limit, control or inhibit the amount of a viral nucleic acid. In additional particular methods embodiments, treatment methods include an amount of an antibody composition, subsequence or portion thereof sufficient to increase, induce, enhance, augment, promote or stimulate an immune response against a virus; increase, induce, enhance, augment, promote or stimulate viral clearance or removal; or decrease, reduce, inhibit, suppress, prevent, control, or limit transmission of a particular virus to a subject (e.g., transmission from a host to a subject). In further particular methods embodiments, treatment methods include an amount of an antibody composition, subsequence or portion thereof sufficient to protect a subject from a viral infection or pathology, or reduce, decrease, limit, control or inhibit susceptibility to viral infection or pathology.

Methods of the present disclosure include treatment methods, which result in any therapeutic or beneficial effect. In various methods embodiments, viral infection, proliferation or pathogenesis is reduced, decreased, inhibited, limited, delayed or prevented, or a method decreases, reduces, inhibits, suppresses, prevents, controls or limits one or more adverse (e.g., physical) symptoms, disorders, illnesses, diseases or complications caused by or associated with a viral infection, proliferation or replication, or pathology. In additional various particular embodiments, treatment methods include reducing, decreasing, inhibiting, delaying or preventing onset, progression, frequency, duration, severity, probability or susceptibility of one or more adverse symptoms, disorders, illnesses, diseases or complications caused by or associated with a particular viral infection, proliferation or replication, or pathology. In further various particular embodiments, treatment methods include improving, accelerating, facilitating, enhancing, augmenting, or hastening recovery of a subject from a particular viral infection or pathogenesis, or one or more adverse symptoms, disorders, illnesses, diseases or complications caused by or associated with a viral infection, proliferation or replication, or pathology. In yet additional various embodiments, treatment methods include stabilizing infection, proliferation, replication, pathogenesis, or an adverse symptom, disorder, illness, disease or complication caused by or associated with a viral infection, proliferation or replication, or pathology, or decreasing, reducing, inhibiting, suppressing, limiting or controlling transmission of a virus from and to an uninfected subject.

A therapeutic or beneficial effect of treatment is therefore any objective or subjective measurable or detectable improvement or benefit provided to a particular subject. A therapeutic or beneficial effect can but need not be complete ablation of all or any particular adverse symptom, disorder, illness, disease or complication caused by or associated with a viral infection, proliferation or replication, or pathology. Thus, a satisfactory clinical endpoint is achieved when there is an incremental improvement or a partial reduction in an adverse symptom, disorder, illness, disease or complication caused by or associated with a viral infection, proliferation or replication, or pathology, or an inhibition, decrease, reduction, suppression, prevention, limit or control of worsening or progression of one or more adverse symptoms, disorders, illnesses, diseases or complications caused by or associated with a particular viral infection, viral numbers, titers, proliferation or replication, viral protein or nucleic acid, or viral pathology, over a short or long duration (hours, days, weeks, months, etc.).

A therapeutic or beneficial effect also includes reducing or eliminating the need, dosage frequency or amount of a second active such as another drug or other agent (e.g., anti-viral) used for treating a subject having or at risk of having a viral infection or pathology. For example, reducing an amount of an adjunct therapy, for example, a reduction or decrease of a treatment for a viral infection or pathology, or a vaccination or immunization protocol is considered a beneficial effect. In addition, reducing or decreasing an amount of a viral antigen used for vaccination or immunization of a subject to provide protection to the subject is considered a beneficial effect.

Adverse symptoms and complications associated with a viral infection and pathology include, but are not limited to for example, e.g., fever, rash, headache, cough, shortness of breath or difficulty breathing, chills (including shaking associated with chills), sore throat, loss of taste or smell, tiredness, difficulty breathing, pain behind the eyes, conjunctivitis, muscle or joint pain, nausea, vomiting, loss of appetite, or secondary infection. Other symptoms of a viral infection or pathogenesis are known to one of skill in the art and treatment thereof in accordance with the invention is provided. Thus, the aforementioned symptoms and complications are treatable in accordance with the present disclosure.

Adverse symptoms and complications associated with viral infections and pathologies can include, but are not limited to, for example, e.g., fever, rash, headache, cough, tiredness, difficulty breathing, pain including pain behind the eyes, stomach pain, muscle or joint pain, weakness, fatigue, conjunctivitis, nausea, vomiting, diarrhea, loss of appetite, hemorrhaging, bleeding, bruising, or secondary infection. Other symptoms of viral infections or pathogenesis are known to one of skill in the art and treatment thereof in accordance with the invention is provided. Thus, the aforementioned symptoms and complications are treatable in accordance with the present disclosure.

Methods, uses and compositions of the present disclosure include administration of the antibody compositions to a subject prior to contact, exposure or infection by a particular virus, administration prior to, substantially contemporaneously with or after a subject has been contacted by, exposed to or infected with a particular virus, and administration prior to, substantially contemporaneously with or after a particular virus pathology or development of one or more adverse symptoms, disorders, illness or diseases caused by or associated with a particular viral infection, or pathology. A subject infected with a particular virus may have an infection over a period of 1-5, 5-10, 10-20, 20-30, 30-50, 50-100 hours, days, months, or years.

Compositions and uses and methods of the present disclosure can be combined with any compound, agent, drug, treatment or other therapeutic regimen or protocol having a desired therapeutic, beneficial, additive, synergistic or complementary activity or effect.

Combination methods and use embodiments include, for example, second actives such as anti-pathogen drugs, such as protease inhibitors, reverse transcriptase inhibitors, virus fusion inhibitors and virus entry inhibitors, antibodies to pathogen proteins, live or attenuated pathogen, or a nucleic acid encoding all or a portion (e.g., an epitope) of any protein or proteinaceous pathogen antigen, immune stimulating agents, etc., and include contact with, administration in vitro or in vivo, with another compound, agent, treatment or therapeutic regimen appropriate for pathogen infection, vaccination or immunization.

Methods of the present disclosure also include, among other things, methods that result in a reduced need or use of another compound, agent, drug, therapeutic regimen, treatment protocol, process, or remedy. For example, for a particular viral infection or pathology, vaccination or immunization, a method of the present disclosure has a therapeutic benefit if in a given subject a less frequent or reduced dose or elimination of an anti-viral treatment results. Thus, in accordance with the invention, methods of reducing need or use of a treatment or therapy for a particular viral infection or pathology, or vaccination or immunization, are provided.

Methods of the present disclosure include methods of diagnosing a viral infection in a subject. In embodiments, the viral infection comprises an infection with coronavirus. In an aspect, the method of diagnosing a viral infection comprises obtaining a biological sample from a subject and contacting the biological sample with any of the antibody compositions described herein. In embodiments, the biological sample comprises a tissue sample from a subject. In embodiments, the biological sample comprises a blood sample from a subject. In embodiments, the biological sample derives from a nasal swab of a subject. In embodiments, the biological sample derives from a throat swab of a subject.

Methods of the present methods include methods of detecting a viral protein. In embodiments, the method comprises detecting a coronavirus protein. In embodiments, the method comprises detecting a coronavirus nucleoprotein.

In certain aspects, the coronavirus is SARS, MERS, 229E (alpha), NL63 (alpha), OC43 (beta), HKU1 (beta), SARS-CoV-2, or an emerging variant thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), Delta (B.1.617.2 and AY lineages), and Omicron (B.1.1.529) or a variant (including but not limited to BA.1, BA.2, or BA.3) thereof.

In embodiments, the methods of diagnosing a viral infection and the methods of detecting a viral protein comprise contacting the sample with any of the antibody compositions described herein. In embodiments, the antibody compositions described herein comprise at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one or more of SEQ ID NOs: 1-392. In embodiments, the antibody compositions described herein comprise any one or more of SEQ ID NOs: 1-392.

In embodiments, the antibody compositions described herein comprise a detectable moiety. Examples of the detectable moieties include but are not limited to a radiolabel, a fluorescent label, an enzymatic label, biotin, chromophore, or an ECL label.

In embodiments, the methods of diagnosing a viral infection and methods of detecting a viral protein comprise using antibody-based assays. In embodiments, the antibody-based assay is an ELISA assay. In embodiments, the antibody-based assay is a radioimmunoassay. In embodiments, the antibody-based assay is fluoroimmunoassay. In embodiments, the antibody-based assay is a chemiluminescenceimmunoassay.

In an aspect, the present disclosure also provides diagnostic kits comprising any one or more of any of the antibody compositions described herein. In embodiments, the antibody compositions described herein comprise at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or greater than 95% sequence identity with any one or more of SEQ ID NOs: 1-392. In embodiments, the antibody compositions described herein comprise any one or more of SEQ ID NOs: 1-392.

In embodiments, the antibody compositions in the kit bind to a protein of a virus. In embodiments, the antibody compositions in the kit bind to a protein of a coronavirus. In embodiments, the antibody compositions in the kit bind to a nucleoprotein of a coronavirus. In embodiments, the antibody compositions in the kit contain a detectable moiety. In embodiments, the detectable moiety comprises a radiolabel, a fluorescent label, an enzymatic label, biotin, chromophore, or an ECL label. In certain aspects, the coronavirus is SARS, MERS, 229E (alpha), NL63 (alpha), OC43 (beta), HKU1 (beta), SARS-CoV-2, or an emerging variant thereof. SARS-CoV-2 variants include the Wuhan parental sequence with or without the D614G mutation, Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), Mu (B.1.621, B.1.621.1), Zeta (P.2), Delta (B.1.617.2 and AY lineages), and Omicron (B.1.1.529) or a variant (including but not limited to BA.1, BA.2, or BA.3) thereof.

In embodiments, the diagnostic kit contains primary antibodies and secondary antibodies. In embodiments, the primary antibodies comprise any of the antibody compositions described herein. In embodiments, the secondary antibodies are capable of binding the primary antibodies and contain a suitable detectable label.

Without limiting any of the foregoing, the following exemplification of carriers, modes of administration, dosage forms, etc., are listed as known possibilities from which the carriers, modes of administration, dosage forms, etc., may be selected for use with the present invention. Those of ordinary skill in the art will understand, however, that any given formulation and mode of administration selected should first be tested to determine that it achieves the desired results.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical and intradermal routes. Administration can be systemic or local.

The compositions of the present disclosure may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen free water, before use.

For instance, the composition of the present disclosure may be administered in the form of an injectable preparation, such as sterile injectable aqueous or oleaginous suspensions. These suspensions may be formulated according to techniques known in the art using suitable dispersing or wetting agents and suspending agents. The sterile injectable preparations may also be sterile injectable solutions or suspensions in non-toxic parenterally-acceptable diluents or solvents. They may be given parenterally, for example intravenously, intramuscularly or sub-cutaneously by injection, by infusion or per os. Suitable dosages will vary, depending upon factors such as the amount of each of the components in the composition, the desired effect (short or long term), the route of administration, the age and the weight of the subject to be treated. Any other methods well known in the art may be used for administering the composition of the present disclosure.

The antibody compositions of the present disclosure may be formulated as a dry powder (i.e., in lyophilized form). Freeze-drying (also referred to as lyophilisation) is often used for preservation and storage of biologically active material because of the low temperature exposure during drying. Lyophilizing the composition can result in a more stable composition.

In certain embodiments, the composition of the present disclosure may be formulated as a liquid (e.g., aqueous formulation), e.g., as syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid).

Where the composition of the present disclosure is intended for delivery to the respiratory (e.g., nasal) mucosa, typically it is formulated as an aqueous solution for administration as an aerosol or nasal drops, or alternatively, as a dry powder, e.g., for rapid deposition within the nasal passage. Compositions for administration as nasal drops may contain one or more excipients of the type usually included in such compositions, for example preservatives, viscosity adjusting agents, tonicity adjusting agents, buffering agents, and the like. Viscosity agents can be microcrystalline cellulose, chitosan, starches, polysaccharides, and the like. Compositions for administration as dry powder may also contain one or more excipients usually included in such compositions, for example, mucoadhesive agents, bulking agents, and agents to deliver appropriate powder flow and size characteristics. Bulking and powder flow and size agents may include mannitol, sucrose, trehalose, and xylitol.

### Tables

Tables 1 and 2 report binding characteristics of certain antibody compositions described herein.

TABLE 1
Novel anti-N antibodies, RNA binding domain-specific

| | | | | | | |
|---|---|---|---|---|---|---|
| | N | N olig. | N RNA-bind. | Kd full- | Kd olig. | Kd RNA-b |
| mAb name | full-length | domain | domain | length | domain | dom. |

| | | | | | | |
|---|---|---|---|---|---|---|
| NP3-A11 | X | X | 0.71 | nM | 1.01 | nM |
| NP3-B1 | X | X | 2.01 | nM | <1 | pmol |
| NP3-B2 | X | X | 1.51 | nM | 0.90 | nM |
| NP3-B9 | X | X | 0.38 | nm | 0.47 | nM |
| NP3-C5 | X | X | 0.73 | nM | 0.62 | nM |
| NP3-C10 | X | X | 1.05 | nM | 2.36 | nM |
| NP3-D1 | X | X | 5.63 | nM | 1.5 | pM |
| NP3-D5 | X | X | <1 | pmol | <1 | pmol |
| NP3-E2 | X | X | 0.57 | nM | <1 | pmol |
| NP3-E3 | X | X | <1 | pmol | 1.36 | nm |
| NP3-E4 | X | X | 0.71 | nM | 3.46 | nM |
| NP3-E5 | X | X | 0.80 | nm | 1.38 | nM |
| NP3-E11 | X | X | 1.23 | nM | 0.57 | nM |
| NP3-F4 | X | X | 2.84 | nM | 5.57 | nM |
| NP3-F10 | X | X | 2.53 | nM | 5.60 | nM |
| NP3-G5 | X | X | <1 | pmol | <1 | pmol |
| NP3-G6 | X | X | 0.43 | nM | 0.88 | nM |
| NP3-H9 | X | X | 5.2 | nM | 3.16 | nM |
| NP3-H12 | X | X | 0.72 | nM | 1.89 | nM |

TABLE 2
Novel anti-N antibodies, full-length-specific and oligomerization domain-specific

| | | | | | | |
|---|---|---|---|---|---|---|
| | N | N olig. | N RNA-bind. | Kd Full- | Kd olig. | Kd RNA-b |
| mAb name | full-length | domain | domain | length | domain | dom. |

| | | | | | | |
|---|---|---|---|---|---|---|
| NP3-A7 | X | | 2.75 | nM | | |
| NP3-C9 | X | | 0.75 | nM | | |
| NP3-B4 | X | X | 1.68 | nM | <1 | pmol |
| NP3-B12 | X | X | 1.00 | nM | 0.78 | nM |
| NP3-C10 | X | X | 2.57 | nM | 0.08 | nM |
| NP3-E6 | X | X | 0.48 | nM | <1 | pmol |
| NP3-F7 | X | X | <1 | pmol | <1 | pmol |
| NP3-G8 | X | X | 2.54 | nM | 1.05 | nM |
| NP3-G9 | X | X | <1 | pmol | <1 | pmol |
| NP3-G12 | X | X | <1 | pmol | <1 | pmol |

### EXAMPLES

### Example 1. ELISA Binding Data

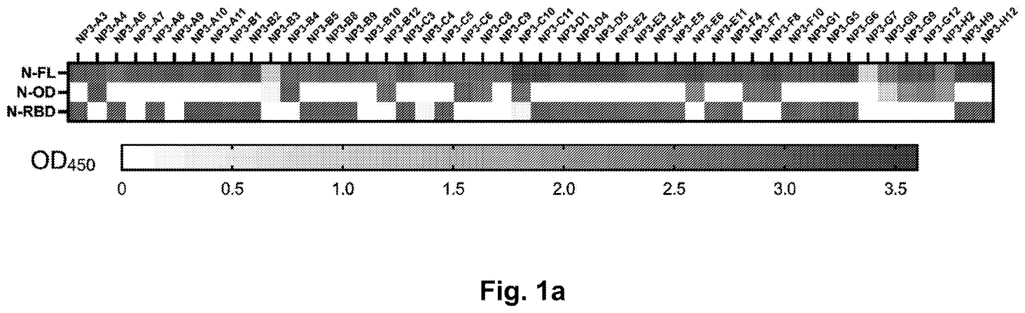

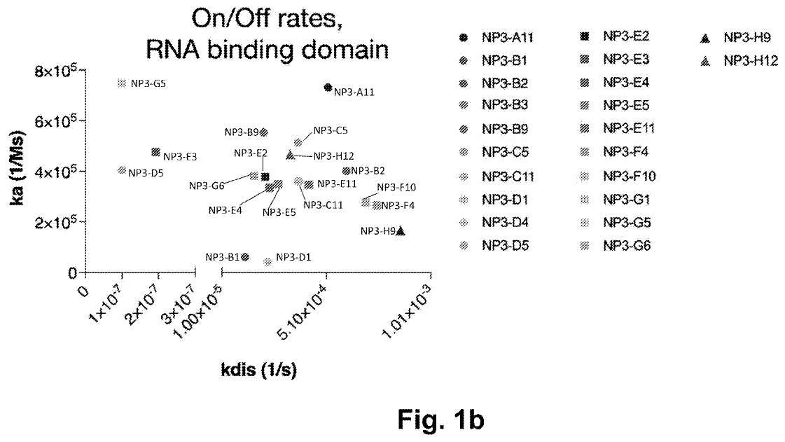

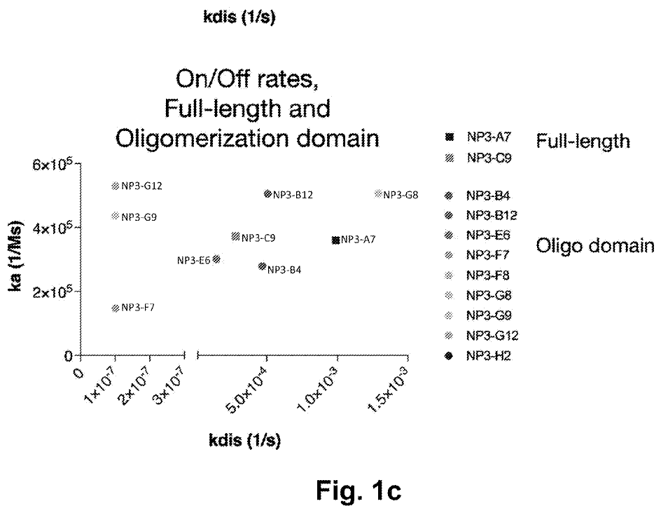

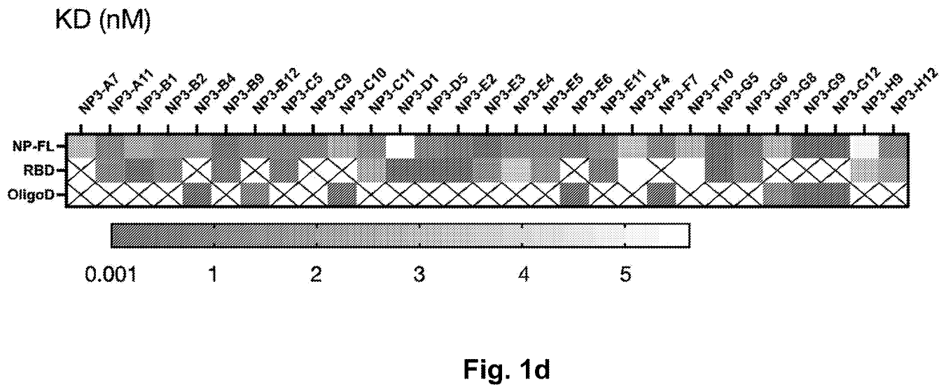

SARS-CoV-2 N protein was coated on ELISA microtiter plates. Supernatants from small-scale (2 mL) antibody test expressions was captured overnight and binding analyzed with an anti-mouse IgG-HRP secondary. FIGS. 1B-1D show kinetic analysis for a subset of the antibodies in FIG. 1a. “X” indicates no binding in Octet analysis. FIG. 1b shows kinetic analysis of RNA-binding domain (RBD) targeted antibodies. The association constant (ka) is plotted against the dissociation constant (kdis). Three antibodies (NP3-D5, NP3-E3 and NP3-G5) have particularly high affinity (fast association, slow dissociation) for the RBD of N. FIG. 1c shows kinetic analysis of N antibodies targeting the oligomerization domain (OD) and those that bind only to the full-length N protein. Three OD-targeted antibodies (NP3-F7, NP3-G9 and NP3-G12) are of particularly high affinity. FIG. 1d shows affinity constants (KD) for anti-N antibodies. These data are summarized in FIGS. 1a-1d and Tables 1 and 2.

Heavy chain sequences corresponding to the tested sequences are described below:

### Heavy Chains

NP3_A3
DVQLQESGPELKKPGETVKISCKASGYTFTDYAIHWVKQAPGKGLKWMGWINTETGEP
KYAEDFKGRFAFSLETSVSTAYLQSNNLKNEDTATYFCARHYRYDEAWFAYWGRGTL
VTVSS (SEQ ID NO: 1)
NP3_A4
QVTLKESGPGILKPSQTLSLTCSFSGFSLSTSGMGVGWIRQPSGKGLEWLAHIWWDDDK
YHNPSLKSQLTISKDTSRNQVFLKITSVDTADTATYYCARSGYRGNFDYWGQGTTVTVS
S (SEQ ID NO: 2)
NP3_A6
QIQLVQSGPELKKPGETVKIACKASGYTFTDYSMNWVKQAPGKGLKWMGWINTETGEP
TYEDDFKGRFAFSLETSASTAYLQITNLKNEDTATYFCVRGYRYDEAWFVWGQGTLVT
VSA (SEQ ID NO: 3)
NP3_A7
QIQLVQSGAELVKPGASVKLSCTASGFNIKDTYIHWVRQRPEQGLEWIGRIDPANGDTK
YDPKFQDKATLIADTSSNTAYLQLGSLTSEDTAVYYCARYRIYYFDYWGQGTTLTVSS
(SEQ ID NO: 4)
NP3_A8
QVQLQQSGAELVKPGASVKLSCKASGYTFTSYYMYWVKQRPGQGLEWIGEINPSNGGF
SFNEKFKSKATLTVDKSSSTAYMQLTSLTSEDSAVYYCTRLEGYGDSDYAMIYWGQGT
TVTVSS (SEQ ID NO: 5)
NP3_A9
EVQLVESGAELVKPGASVKISCKASGYTFTDYNMDWVKQSHGKSLEWIGDINPDYGST
SYNQKFRGKATLTVDKSSSTANMELRSLTSEDTAVYYCARGDYDGGYGMDYWGQGTS
VTVSS (SEQ ID NO: 6)
NP3_A10
QVQLQQSGPSLVKPSQTLSFTCSVTGDSVTSGYWNWIRKFPGNKLEFMGYISYSGSTYY
SPSLKSRISIIRDTSKNRYYLQLNSVTTEDTATYFCARGLRFFYAMDYWGQGTTLTVSS
(SEQ ID NO: 7)
NP3_A11
EVQLQESGPELKKPGETVKISCKVSGYTFTNYGMNWVKQAPGKGLKWMGWINTYTGE
PKYGDDFKGRFALSLETSAGTAYLRINNLKNEDTATYFCARSHGNYENHFDYWGQGTT
LTVSS (SEQ ID NO: 8)
NP3_B1
QVQLQQSGPELVKPGTSVTVYCKASGYTFTTYNIYWVKQSHGKSLEWIGYIDPYNGGTS
YNQKFKGKATLTVDKSSSAAYMHLNSLTSEDSAVYYCALPWDYWGQGTTVTVSS
(SEQ ID NO: 9)
NP3_B2
QIQLVQSGGGLVKPGGSLKLSCAASGFTFSDYYMSWVRQTPEKRLEWVATISDGGSYT
YYPDSVKGRFTISRDNAKNNLYLHMSSLKSEDTAMYYCARGSTYGNPLDYWGQGTTL
TVSS (SEQ ID NO: 10)
NP3_B3
EVQLQQSGTVLARPGASVKMSCKASGSSFTTYWMHWVKQRPGQGLEWIGAIYPGNSD
TSYNQKFKGKAKLTAVTSASTAYMELSSLTNEDSAVYYCTRKELFGYGFDYWGQGTTL
TVSS (SEQ ID NO: 11)
NP3_B4
EVQLVESGGGLVQPGGSVKLSCLASGFTFSDYYMSWVRQSPEKGLEWVAEIRLESDNY
ATHYAESVKGKFTISRDDSKSRLYLKMNSLRGEDTGIYYCAFDVYYGGAMDYWGQGT
TVTVSS (SEQ ID NO: 12)
NP3_B5
QVQLQQSGAELVRPGASVKLSCKASGYTFTSYWMNWVKQRPEQGLAWIGRIDPYDNA
THYNHKFKDKAILTVDKSSSTAYMELRGLTSEDSAVYYCAIYYDYDDYIMDYWGQGTS
VTVSS (SEQ ID NO: 13)
NP3_B8
DVQLQESGGGLVKPGGSLKLSCAASGFTFSDYYMYWVRQTPERRLEWVATISDGGSYT
YYPDSVKGRFTSSRDNAKNNLYLQMSSLKSEDTAMYYCTRGDDYGGRLAYWGQGTL
VTVSA (SEQ ID NO: 14)
NP3_B9
EVQLQQSGPELKKPGETVKISCKTSGYTFTNCGMHWVKQAPGKGLKWMGWINTYTGE
PTYADDFKGRFAFSLETSVNTAYLQINNLKSEDTASYFCARRDDVYYVAGFDYWGQGT
TLTVSS (SEQ ID NO: 15)
NP3_B10
QIQLVQSGPELKKPGETVKISCKASGYTFTDYSMHWVKQAPGKDLKWVGWINTETGEP
TYADDFKGRFAFSLETSATTAYLQINNLKNEDTATYFCARGAATRYWGQGTTLTVSS
(SEQ ID NO: 16)
NP3_B12
EVQLQQSGAELMKPGASVKISCKATGYTFSSYWIEWVKQRPGHGLEWIGEFFPGSGNTN
YNEKFKGKATFTADTSSNTAYMQLSSLTSEDSAVYYCARRGLYDGYYVRYFDVWGAG
TTVTVSS (SEQ ID NO: 17)
NP3_C3
QIQLVQSGPELRKPGETVKISCKASGYIFTNYGMNWVKQAPGKGLKWMGWINTYTGEP
TYAGDFKGRFAFSLETSASTAYLQINNLKNEDSATYFCARSHDNYDNHFDYWGQGTTL
TVSS (SEQ ID NO: 18)
NP3_C4
QIQLVQSGPELVKPGASVKISCKASGYSFTNYYMHWVRQSHVKSLEWIGRINPYSGATS
YNQHFKDKASLTVDKSSSTAYMELHSLTSEDSAVYYCADNGYLWGQGTSVTVSS (SEQ
ID NO: 19)
NP3_C5
QIQLVQSGPELKTPGETVKISCKASGYTFTNCGMHWVKQAPGKGLKWMGWINTYTGEP
IYADDFKRRFAFSLETSASTAYLQINNLNNEDTASYFCARRDDVYYVAGFDYWGQGTT
VTVFS (SEQ ID NO: 20)
NP3_C6
QVQLQQSGPELVKPGASVKMSCKASGYTFTDYVINWVKERTGQGLEWIGEIYPGSGDT
YYNEKFKGKATLTADKSSNTAYMHLSSLTSEDSAVFFCARRKFYDGNYVRYFDVWGA
GTTLTVSS (SEQ ID NO: 21)
NP3_C8
EVQLQQSGAELARPGASVKMSCKASGYTFTSYTMHWVKQRPGQGLEWLGYFNPSSGY
TNYVQRFRDKATLTADKSSSTAYMQLSSLTSEDSAVYYCARAGEGYAMDYWGQGTSV
TVSS (SEQ ID NO: 22)
NP3_C9
QIQLVQSGPELKKPGETVKISCKASGYTFTDYSIHWVKQAPGKDLKWVGWINTETGEPT
YADDFEGRFAFSLETSASTAFLQINNLKNEDTATYFCSRGAATGFWGQGTTLTVSS (SEQ
ID NO: 23)
NP3_C10
QVTLKESGPGILKPSQTLSLTCSFSGFSLSTSGLGVGWIRQPSGKGLEWLAHIWWDDDK
YYNPSLKSQLTISKETSRNQVFLKISSVDSADTATYYCARTVYHGAMDYWGQGTSVTV
SS (SEQ ID NO: 24)
NP3_C11
EVQLVESGGGLVRPGGSLKLSCAASGFTFSDYYMYWVRQTPEKRLEWVATISDGGGYN
YYPDSVKGRFTVSRDNAKNNLYLQMSSLKSEDTAMYYCVRDKYDGWGSDFDVWGAG
TTVTVSS (SEQ ID NO: 25)
NP3_D1
DVQLQESGPELAKPGASVKVSCKASGYTFTSYNIYWVKQSHGKSLEWIGYIDPYNGGTS
YNQKFKGKATLTVDKSSSTAYMHLNSLTSEDSAVYYCALPWDYWGQGTSVTVSS (SEQ
ID NO: 26)
NP3_D4
EVQLVESGPSLVKPSQTLSLTCSVTGDSITSGYWNWIRKFPGNKFEFMGYMRYSGSTYF
SPSLKSRISITRDTSKNHYYLQLSSVTTEDTATYYCARGLRFFYAMDYWGQGTSVTVSS
(SEQ ID NO: 27)
NP3_D5
DVQLQESGPELVRPGVSVKISCKGSGYTFTDYVMHWVKQSHAKSLEWIGVISTYSGNT
NYNQKFKGKATMTVDKFSNTAYMELARLTSEDSAIYYCTRGTGDFDYWGQGTTLTVSS
(SEQ ID NO: 28)
NP3_E2
EVQLQQSGPEVVRPGVSVKISCKGSGYTFTDYALHWVKQSHTKSLEWIGVISTYSGTTN
YNQNFKGKATMAVDKSSSTAYMELARLTSEDSAIYYCASGGNYGLDYWGQGTSVTVS
S (SEQ ID NO: 29)
NP3_E3
QIQLVQSGPDLKKPGETVKISCKASGYTFTDYSMHWVKQVPGKGLKWMGWINTETDK
PTYADDFKGRFAFSLETSASTAYSQINNLKNEDTATYFCARHYKYDEAWSPYWGQGTL
VTVSA (SEQ ID NO: 30)
NP3_E4
EVQLVESGPGLVAPSQSLSITCTVSGFSLTSYGVSWVRQPPGKGLEWLGVIWGDGSTNY
HSALISRLSINKDNSKSQVFLRLNSLQTDDTATYYCAKEDDGYLFYFDYWGQGTTLTVS
S (SEQ ID NO: 31)
NP3_E5
EVQLVESGGGLVKPGGSLKLSCAASGFTFSDYYMSWVRQTPEKRLEWVATISDGGNYT
YYPDSVKGRFTISRDNAKNNLYLQMSSLKSEDTAMYYCARGSTYGNPLDHWGQGTTL
TVSS (SEQ ID NO: 32)
NP3_E6
EVQLQQSGAELMRPGASVKISCKATGYTFSSYWIEWVKQRPGHGLEWIGEILPGSDNTN
YNEKFKGKATLTADTSSNTAYMHLSSLTSEDSAVYSCARRLFLRRYFDVWAGTTVTVS
S (SEQ ID NO: 33)
NP3_E11
EVQLQQSGPELVRPGVSVKISCKGSGYTFTDYVMHWVKQSHAKSLEWIGVISTYSGNT
NYNQKFKGKATMTVDKSSNTVYMELARLTSEDSAIYYCTRGTGDFDYWGQGTTLTVSS
(SEQ ID NO: 34)
NP3_F4
EVQLVESGPELKKPGETVKIPCKTSGYTFTNYGMNWVKQAPGKGLKWMGWINTNTGE
PTYAEDFKGRFAFSLETSASTAYLQISNLKNEDTATFFCARGSIYDGYYEGLYYWGQGT
TLTVSS (SEQ ID NO: 35)
NP3_F7
EVQLQQSGAELVRPGSSVKISCKASGYAFSSYWMNWVKQRPGQGLEWIGQIYPGNGDT
YYNGKFMGKATLTADKFSSTAYMQLSGLTSEDSAVYFCARRPLFYAMDYWGQGTSVT
VSS (SEQ ID NO: 36)
NP3_F8
QVQLQQSGHGLVAPSQSLSITCTVSGFSLSSYGVHWVRQPPGKGLEWLAVIWSDGNTM
YNSVLKSRLNISRDYSKSQVFLKGNSLQTDDTAMYYCARNSGYNDYDGYYFDVWGAG
TSVTVSS (SEQ ID NO: 37)
NP3_F10
EVQLQQSGAELVEPGAPVKLSCKAFGYTFTSYWMNWVKQRPGRGLEWIGRIDPSDSET
HYNQNFKDKAALTVDRSSSTAYIHLSSLTSEDSAVYYCAREVTTATKGVFSWLAYWGQ
GTLVTVSA (SEQ ID NO: 38)
NP3_G1
EVQLVESGAELVKPGASVKLSCKASGYTFTSYYMYWVQLRPGQGLEWIGEINPSNGGT
NFNEKFKSKATLTVDKSSSTAYMQLSSLTSEDSAVYYCTRLEGYGDYDYAMYYWGQG
TSVTVSS (SEQ ID NO: 39)
NP3_G5
EVQLVESGPELKKPGEPVRISCKASGYTFTDYGMNWVKQAPGKGLKWMGWINTYTGE
PTYADDFKGRFAFSLETSASTAFLQITNLKNEDTATYFCARAPLLRSYFDYWGQGTSVT
VSS (SEQ ID NO: 40)
NP3_G6
EVQLVESGGGLVKPGGSLKLSCAASGFTFSDYYMSWVRQTPEKRLEWVATISDGGSYT
YYPDSVKGRFTISRDNAKNNLYLHMSSLKSEDTAMYYCARGSTYGNPLDYWGQGTSV
TVSS (SEQ ID NO: 41)
NP3_G7
QIQLVQSGAELVRPGSSVKISCKASGYAFSSYWMNWVKQRPGQGLEWIGQIYPGNGDT
YYNGKFMGKATLTADKFSSTAYMQLSSLTSEDSTVYFCARRPLFYAMDYWGQGTSVT
VSS (SEQ ID NO: 42)
NP3_G8
QVQLQQSGPELKKPGETVKISCKASGYTFTDYSMHWVKQAPGKGLKWLGWKNTESGE
ETFADDFKGRFDFSLETSASTVYLQIKNLKNEDTAAYFCARSGDIYAMDYWGQGTTVT
VSS (SEQ ID NO: 43)
NP3_G9
EVQLQQSGAELVRPGSSVKISCKASGYAFSNYWMNWVRQRPGQGLEWIGQIYPGNDDT
YYNGKFKGKVTLTADKSSTTAYIQLISLTSEDSAVYFCARRPLFYAMDYWGQGTSVTVS
S (SEQ ID NO: 44)
NP3_G12
EAQLQQSGAEPMKPGASVKISCKTTGYTFTSDWIEWVQQRPGHGLEWIGEILPGSGNTN
YNENFKGKATLTADTSSNTAYMHLSSLTSEDSAVYYCARRLFLRRYFDVWGAGTTVTV
SS (SEQ ID NO: 45)
NP3_H2
EVQLQQSGAELARPGASVKMSCKASGYIFTSYTIHWIKQRPGQGLEWIGYFNPSSDYFS
YIQIFKDKATLTADKFSSTAYMQLSSLTSEDSAVYYCARAGEVYAKDYWGQGTSVTVS
S (SEQ ID NO: 46)
NP3_H9
QIQLVQSGPELKKPGETVKISCKASGYTFTDYPIHWVKQAPGKGLKWMGWINAETGEPT
YADDFKGRFVVSLETSASTAYLQINNLKNEDTATFFCARHYRYDEAWFVYWGQGTLVT
VSA (SEQ ID NO: 47)
NP3_H12
QIQLVQSGPELKKPGETVKISCKASGYMFTNYGMNWVKQAPGKGLKWMGWINTYTGE
PTYADDFKGRFAFSLDISGSTAYLQINNLKNEDMATYFCARTREIWGNVDFPYWGQGTL
VTVSA (SEQ ID NO: 48)
Light chains corresponding to the tested sequences are described below:
Light Chains
NP3_A3
DIVMTQSPASLSVSVGETVTITCRSSENIYSNLAWFQQKQGKSPQLLVYGATDLADGVPS
RFSGSGSGTQYSLKINSLQSEDFGTYYCQHFWGTPWTFGGGTKLEIK (SEQ ID NO: 49)
NP3_A4
DIVMTQSHKFMSTSVGDRVSITCKASQDVSTAVAWYQQKPGQSPKLLIYSASHRYTGVP
DRFTGSGSGTDFTFTISSVQAEDLAVYYCQQHYSNPLTFGAGTKLEIK (SEQ ID NO: 50)
NP3_A6
DIQMNQSPSSLSVSVGETVTITCRTSENIYSNLAWYQQKQGKSPQLLVYGATDLADGVP
SRFSGSGSGTQYSLKISSLQSEDFGTYYCQHFWGTPWTFGGGTKLEIK (SEQ ID NO: 51)
NP3_A7
DIQMTQSPASVPVTPGESVSISCRSSKSLLHSNGNTYVYWFLQRPGQSPRLLIYRMSNLA
SGVPDRFSGSGSGTAITLRISRVEAEDVGVYSCMQHLEYPLTFGAGTKLEIK (SEQ ID NO:
52)
NP3_A8
DIVMTQSPASLVVSLGQRATISCRASKSVSASGYSYIHWYQQIPGQPPKLLIYLASNLESG
VPARFSGSGSGTDFTLNIHPVEEEDAATYYCQHSRELPLTFGTGTKLEIK (SEQ ID NO: 53)
NP3_A9
ENVLTQSPASNPVTLGTSASISCRSSKSLLHSNGFTYLYWYLQKPGQSPQLLIYQMSYLA
SGVPDRESSSGSGTDFTLRISRVEAEDVGVYYCAQNLELPFTFGSGTKLEIK (SEQ ID NO:
54)
NP3_A10
ENVLTQSPASLTVSLGQRATLSCKASQSVDYDGDSYMNWYQQKPGQPPKLLIYAASYL
QSGIPARFSGSGSGTDFTLNIHPVEEEDAATYYCQQSIEDPYTFGGGTKLEIK (SEQ ID
NO: 55)
NP3_A11
DIQMTQSPSSLSVSVGETVTITCRASENIYSSLAWYQQKQGKSPQLLVYTATDLADGVPS
RFSGSGSGSQYSLRINSLQSEDFGSYYCQHFWGFPWTFGGGTKLEIK (SEQ ID NO: 56)
NP3_B1
DVVMTQTPLTLSVTIGQPASISCKSSQTLLDSDGKTYLNWLFQRPGQSPKRLIYLVSELD
SGVPDRFTGSGSGTDFTLKISRVEAEDLGVYYCWQGTHFPQTFGGGTKLELK (SEQ ID
NO: 57)
NP3_B2
DIQMTQTTSSLSASLGDRVTINCSASQGISNYLNWYQQKPDGTVKLLIYFTSSLHSGFPSR
FSGSGSGTDFSLTISNLEPEDIATYYCQQYSKLPFTFGSGTKLEIK (SEQ ID NO: 58)
NP3_B3
DIVLTQSPASLAVSLGQRATISCKASQSVDYDGDSYMNWYQQKPGQPPKLLIYAASNLE
SGIPARFSGSGSGTDFTLNIHPVEEEDAATYYCQQSNEDPYTFGGGTKLELK (SEQ ID NO:
59)
NP3_B4
DIQMTQSPSIMSASPGEKVTMTCSASSSVSYMHWYQQKSSTSPKLWIYDTSELASGVPG
RFSGSRSGNSYSLTISSMEAEDVATYYCFQGSGYPLTFGGGTKLELK (SEQ ID NO: 60)
NP3_B5
ENVLTQSPAIMSASPGEKVTMTCSASSSVNYMHWYQQRPGSSPKPWIYDTSNLASGIPV
RFSGSGSGTSYSLIISSMEAEDAATYYCHQRSSFPFTFGSGTKLEIK (SEQ ID NO: 61)
NP3_B8
DIQMNQSPSSLSASLGGRVTISCSASQGISNYLNWYQQKPDGTVKLLIYYTSSLHSGVPS
RFSGSGSGTDYSLTISNLEPEDIATYYCQQYNKLPYTFGGGTKLEIK (SEQ ID NO: 62)
NP3_B9
DIQMTQSPASLSVSVGETVTITCRASENIYSNLAWYQQKQGKSPQLLVNGATDLADGVP
SRFSGSGSGTQYSLKINSLQSEDFGSYYCQLFWGTPWTFGGGTKLEIK (SEQ 15ID NO:
63)
NP3_B10
DIVMTQSPSSLAVSLGQRATISCKASQSVDFDGKSYMNWYQQKPGQPPKLLYVASNLES
GIPARFSGSGSGTDFTLNIHPVEEEDAATYYCQQTNEDPLTFGTGTKLEIK (SEQ ID NO:
64)
NP3_B12
DIVLTQSPASLAVSLGQRATISCKASQSVDYDGYSYMNWYQQKPGQPPKLLIYAASNLE
SGIPARFSGSGSGTDFTLNIHPVEEEDAATYYCQQSNADPFTFGGGTKLELK (SEQ ID NO:
65)
NP3_C3
DIVMTQSPASLSVSVGETVTITCRASENIYSNLAWYQQKQGKSPQLLVYVATDLADGVP
SRFSGSGSGTQYSLKINSLQSEDFGSYYCQHFWGTPWTFGGGTKLELK (SEQ ID NO: 66)
NP3_C4
DIVMTQAAPTLSVTIGQPASISCKSSQSLLDSDGKTYLNWLLQRPGQSPRRLIYLVSKLDS
GVPDRFTGSGSGTDFTLKISRVEAEDLGVYYCWQGTHFPRTFGGGTKLEIK (SEQ ID NO:
67)
NP3_C5
DIQMTQSPASLSVSVGETVTITCRASENIYSNLAWYQQKQGKSPQLLVYGATDLADGVP
SRFSGSGSGTQYSLKINSLQSEDFGSYYCQHFWGTPWTFGGGTKLELK (SEQ ID NO: 68)
NP3_C6
DIKMTQSPSSLAVSLGQRATISCKASQSVDYDGDSYMNWYQQRPGQPPKLLIYAASNLE
SGIPARFSGSGSGADFTLNIHPVEEEDAATYYCQQSNEDPWTFGGGTKLEIK (SEQ ID
NO: 69)
NP3_C8
DIVMTQSPASLAVSLGQRATISCKASQSVDYDGDSYMSWYLQKPGQPPKLLIYAASNLE
SGIPARFSGSGSGTDFTLNIHPVEEEDAATYYCQQSIGDPWTFGGGTKLEIK (SEQ ID NO:
70)
NP3_C9
DIVLTQSPASLAVSLGQRATISCKASQSVDYDGDSYMNWYQQKPGQPPKLLIYVASNLE
SGIPARFSGSGSGTDFTLNIHPVEDEDAATYYCQQTNEDPLTFGAGTKLELK (SEQ ID
NO: 71)
NP3_C10
DIKMTQSPSIMSASPGEKVTMTCSASSSVSYMYWFQQKPGSSPRLLIYDTSNLASGVPVR
FSGSGSGTSYSLTISRMEAEDAATYYCQQWSGYPYTFGGGTKLEIK (SEQ ID NO: 72)
NP3_C11
DIVMTQSPSLMSTSLGERVTMTCTANSSVSSNYMHWYQQKPGSSPKLWIYSTSNLASGV
PARFSGSGSGTSYSLTISNMEAEDAATYYCHQFHRSPRTFGGGTKLEIK (SEQ ID NO: 73)
NP3_D1
DIVMTQAAPTLSVTLGQPASISCKSSQSLLDSDGKTYLSWLLQRPGQSPKRLIYLVSELDS
GVPDRFTGSGSGTDFTLKISRVEAEDLGVYYCWQGTHFPQTFGGGTKLEIK (SEQ ID NO:
74)
NP3_D4
ENVLTQSPASLTVSLGQRATLSCKASQSVDYDGDSYMNWYQQKPGQPPKLLIYAASYP
ESGIPARFSGSGSGTDFTLNIHPVEEEDAATYYCQQSIEDPYTFGGGTKLEIK (SEQ ID NO:
75)
NP3_D5
DIVMTQSHKFMSTSVGDRVSITCKASQDVGTAVAWYQQKPGQSPKLLIYWASTRHTGV
PDRFTGSGSGTDFTLTISNVQSEDLADYFCHQYSSYPYTFGGGTKLEIK (SEQ ID NO: 76)
NP3_E2
DIVMTQSHKFMSTSVGDRVSITCKANQDVGSAVAWYQEKPGQSPKLLIYWTSTRHTGV
PDRFTGNGSGTDFTLTISNVQSEDLVDYFCQQYSRYPLTFGSGTKLELK (SEQ ID NO: 77)
NP3_E3
DIVLTQSPASLSVSVGETVTITCRASENIYSNLAWYQQKQGQSPQLLVYAATDLADGVP
SRFSGSGSGTQYSLKINSLQSEDFGNYYCQHFWGTPWTFGGGTKLEIK (SEQ ID NO: 78)
NP3_E4
DIKMTQSPSSLSASLGDRVTISCSASQDISDYLNWYQQKPDGTVKLLIYYTSSLHSGVPSR
FSGSGSGTDYSLTISNLEPEDIATYYCQQYSKLPYTFGGGTKLEIK (SEQ ID NO: 79)
NP3_E5
DIQMTQTTSSLSASLGDRVTINCSASQGISNYLNWYQQKPDGTVKLLIYYTSILHSGVPS
RFSGSGSGTDYSLTISNLEPEDIATYYCQQYSKLPFTFGSGTKLEIK (SEQ ID NO: 80)
NP3_E6
DIVLTQSPASLALSLGQRATISCKASQSVDYDGDSYMNWYQQKPGQPPKLLISAASNLE
SGIPARFGGSGSGTDFTLNIHPVEEEDAATYYCQQSNEDPFTFGGGTKLEIK (SEQ ID NO:
81)
NP3_E11
DIVMTQSHKFMSTSVGDRVSITCKASQDVGSAVAWYQQKPGQSPKLLIYWTSTRHTGV
PDHFTGSGSGTDFTLTINNVQSEDLADYFCHQYSSYPYTFGGGTKLEIK (SEQ ID NO: 82)
NP3_F4
DIKMTQSPSSLSVSVGETVTITCRASENIYSNLAWYQQKQGKSPQLLVSAATNLADNVPS
RFSGSGSGTQYSLKINSLQSEDFGSYYCQHFWGFPRTFGGGTKLEIK (SEQ ID NO: 83)
NP3_F7
DIVLTQSPGSLAVSLGQRATISCKASQSVDYDGDSYMNWYQQKPGQPPKLLIYAASNLE
SGIPVRFSGSGSGTDFTLNIHPVEEEDAATYYCQQSNEDPWTFGGGTKLELK (SEQ ID
NO: 84)
NP3_F8
DIQMTQSPSIMAASVGQKVTMTCSASSSVSSSYLHWYQQKSGASPKPLIHRTSNLASGV
PPRFSGSGSGTSYSLTISSVEAEDDATYYCQQWSGYPFTFGTGTKLEIK (SEQ ID NO: 85)
NP3_F10
IQMTQTTSSLSASLGDRVTISCSASQDISDYLNWYQQKPDGTIKLLIYYTSSLHSGAPSRF
SGSGSGTDYSLTISNLEPEDIATYYCQQYSKLPWTFGGGTKLELK (SEQ ID NO: 86)
NP3_G1
DIVLTQSPASLAVSLGQRATISCRASKSVSASGYSYLHWYQQKPGQPPKLLIYLASNLPS
GVPARFSGSGSGTDFTLNIHPVEEEDAATYYCQHRREIPLTFGAGTKLEIK (SEQ ID NO:
87)
NP3_G5
DIVMTQSHKFMSTSVGDRISITCKASQDVSTAVVWYQQKPGQSLKLLIYSASYRYTGVP
DRFTGSGSGTDFTFTISSVQAEDLAIYYCQQYYSTPFTFGSGTKLEIK (SEQ ID NO: 88)
NP3_G6
DIQMTQSPASLSASLGDRVTINCSASQGISNYLNWYQQKPDGTVKLLIYFTSSLHSGFPSR
FSGSGSGTDFSLTISNLEPEDIATYYCQQYSKLPFTFGSGTKLEIK (SEQ ID NO: 89)
NP3_G7
DIQMTQSPASLSVSVGETVTITCRASENIYSNLAWYQQKQGKSPQLLVYVATDLADGVP
SRFSGSGSDTQYSLKINSLQSEDFGSYYCQHFWGTPWTFGGGTKLEIK (SEQ ID NO: 90)
NP3_G8
ENVLTQSPASLAVSLGQRATISCKASQSVDYDGGSYMNWYQQKPGQPPKLLIYAASTLE
SGIPAGFSGSGSGTDFTLNIHPVEEEDAATYYCQQSNEDPWTFGGGTKLEIK (SEQ ID NO:
91)
NP3_G9
DIQMTQSPSSLAVSLGQRASISCKASQSVDYDGESYMNWYQQKPGQPPKVLIYAASNLE
SGIPARFSGSGAGTDFTLSIHPVEEEDAATYYCQQSYEDPWTFGGGTKLELK
(SEQ ID NO: 92)
NP3_G12
DIVMTQSPASLAVSLGQRATISCKASQSVDYDGDSYMNWYQQKPGQPPKLLIYAASNL
ESGIPARFGGGGSGTDFTLNIHPVEEEDAATYYCQQSNEDPFTFGGGTKLELK (SEQ ID
NO: 93)
NP3_H2
ENVLTQSPASLAVSLGQRATISCKASQSVDYDGDSYMNWYQQKPGQPPKLLIYAASSLD
SGIPARFGGSGSGTDFTLNIHPVEEEDAATYYCQQSNEDPWTFGGGTKLEIK
(SEQ ID NO: 94)
NP3_H9
DVQITQSPSSLSVSVGETVTITCRASENIYSNLAWYQQKQGKSPQLLVYAATDLADGVPS
RFSGSGSGTHYSLKINSLQSEDFGSFYCQHFWGTPWTFGGGTKLEIK (SEQ ID NO: 95)
NP3_H12
DILMTQAAPSLSVSVGETVTITCRASENIYSDLAWYQHKEGKSPQLLVYAATDLADGVP
SRFSGSGSGTQYSLKINSLQSEDFGNYYCQHFWGTPWTFGGGTKLEIK (SEQ ID NO: 96)

### Example 2. CDR Sequences of Heavy Chain and Light Chain Sequences

The antibody sequences described in Example 1 were further analyzed to identify the CDR sequences of the heavy chain and light chain sequences.

### Heavy Chain CDR1 Sequences of SEQ ID NOs: 1-48

Each of the heavy chain sequences of SEQ ID NOs: 1-48 were analyzed to identify the heavy chain CDR1 (CDRH1) sequences. SEQ ID NOs: 97-144 constitute CDRH1 sequences with SEQ ID NOs: 1-48.

NP3_A3
(SEQ ID NO: 97)
GYTFTDYA
NP3_A4
(SEQ ID NO: 98)
GFSLSTSGMG
NP3_A6
(SEQ ID NO: 99)
GYTFTDYS
NP3_A7
(SEQ ID NO: 100)
GFNIKDTY
NP3_A8
(SEQ ID NO: 101)
GYTFTSYY
NP3_A9
(SEQ ID NO: 102)
GYTFTDYN
NP3_A10
(SEQ ID NO: 103)
GDSVTSGY
NP3_A11
(SEQ ID NO: 104)
GYTFTNY
NP3_B1
(SEQ ID NO: 105)
GYTFTTYN
NP3_B2
(SEQ ID NO: 106)
GFTFSDYY
NP3_B3
(SEQ ID NO: 107)
GSSFTTYW
NP3_B4
(SEQ ID NO: 108)
GFTFSDYY
NP3_B5
(SEQ ID NO: 109)
GYTFTSYW
NP3_B8
(SEQ ID NO: 110)
GFTFSDYY
NP3_B9
(SEQ ID NO: 111)
GYTFTNCG
NP3_B10
(SEQ ID NO: 112)
GYTFTDYS
NP3_B12
(SEQ ID NO: 113)
GYTFSSYW
NP3_C3
(SEQ ID NO: 114)
GYIFTNYG
NP3_C4
(SEQ ID NO: 115)
GYSFTNYY
NP3_C5
(SEQ ID NO: 116)
GYTFTNCG
NP3_C6
(SEQ ID NO: 117)
GYTFTDYV
NP3_C8
(SEQ ID NO: 118)
GYTFTSYT
NP3_C9
(SEQ ID NO: 119)
GYTFTDYS
NP3_C10
(SEQ ID NO: 120)
GFSLSTSGLG
NP3_C11
(SEQ ID NO: 121)
GFTFSDYY
NP3_D1
(SEQ ID NO: 122)
GYTFTSY
NP3_D4
(SEQ ID NO: 123)
GDSITSGY
NP3_D5
(SEQ ID NO: 124)
GYTFTDYV
NP3_E2
(SEQ ID NO: 125)
GYTFTDYA
NP3_E3
(SEQ ID NO: 126)
GYTFTDYS
NP3_E4
(SEQ ID NO: 127)
GFSLTSYG
NP3_E5
(SEQ ID NO: 128)
GFTFSDYY
NP3_E6
(SEQ ID NO: 129)
GYTFSSYW
NP3_E11
(SEQ ID NO: 130)
GYTFTDYV
NP3_F4
(SEQ ID NO: 131)
GYTFTNYG
NP3_F7
(SEQ ID NO: 132)
GYAFSSYW
NP3_F8
(SEQ ID NO: 133)
GFSLSSYG
NP3_F10
(SEQ ID NO: 134)
GYTFTSYW
NP3_G1
(SEQ ID NO: 135)
GYTFTSYY
NP3_G5
(SEQ ID NO: 136)
GYTFTDYG
NP3_G6
(SEQ ID NO: 137)
GFTFSDYY
NP3_G7
(SEQ ID NO: 138)
GYAFSSYW
NP3_G8
(SEQ ID NO: 139)
GYTFTDYS
NP3_G9
(SEQ ID NO: 140)
GYAFSNYW
NP3_G12
(SEQ ID NO: 141)
GYTFTSDW
NP3_H2
(SEQ ID NO: 142)
GYIFTSYT
NP3_H9
(SEQ ID NO: 143)
GYTFTDY
NP3_H12
(SEQ ID NO: 144)
GYMFTNYG
Heavy Chain CDR2 Sequences of SEQ ID NOs: 1-48

Each of the heavy chain sequences of SEQ ID NOs: 1-48 were analyzed to identify the heavy chain CDR2 (CDRH2) sequences. SEQ ID NOs: 145-192 constitute CDRH2 sequences within SEQ ID NOs: 1-48.

NP3_A3
(SEQ ID NO: 145)
INTETGEP
NP3_A4
(SEQ ID NO: 146)
IWWDDD
NP3_A6
(SEQ ID NO: 147)
INTETGEP
NP3_A7
(SEQ ID NO: 148)
IDPANGDT
NP3_A8
(SEQ ID NO: 149)
INPSNGGF
NP3_A9
(SEQ ID NO: 150)
INPDYGST
NP3_A10
(SEQ ID NO: 151)
ISYSGST
NP3_A11
(SEQ ID NO: 152)
INTYTGEP
NP3_B1
(SEQ ID NO: 153)
IDPYNGGT
NP3_B2
(SEQ ID NO: 154)
ISDGGSYT
NP3_B3
(SEQ ID NO: 155)
IYPGNSDT
NP3_B4
(SEQ ID NO: 156)
IRLESDNYAT
NP3_B5
(SEQ ID NO: 157)
IDPYDNAT
NP3_B8
(SEQ ID NO: 158)
ISDGGSYT
NP3_B9
(SEQ ID NO: 159)
INTYTGEP
NP3_B10
(SEQ ID NO: 160)
INTETGEP
NP3_B12
(SEQ ID NO: 161)
FFPGSGNT
NP3_C3
(SEQ ID NO: 162)
INTYTGEP
NP3_C4
(SEQ ID NO: 163)
INPYSGAT
NP3_C5
(SEQ ID NO: 164)
INTYTGEP
NP3_C6
(SEQ ID NO: 165)
IYPGSGDT
NP3_C8
(SEQ ID NO: 166)
FNPSSGYT
NP3_C9
(SEQ ID NO: 167)
INTETGEP
NP3_C10
(SEQ ID NO: 168)
IWWDDDK
NP3_C11
(SEQ ID NO: 169)
ISDGGGYN
NP3_D1
(SEQ ID NO: 170)
IDPYNGGT
NP3_D4
(SEQ ID NO: 171)
MRYSGST
NP3_D5
(SEQ ID NO: 172)
ISTYSGNT
NP3_E2
(SEQ ID NO: 173)
ISTYSGTT
NP3_E3
(SEQ ID NO: 174)
INTETDKP
NP3_E4
(SEQ ID NO: 175)
IWGDGST
NP3_E5
(SEQ ID NO: 176)
ISDGGNYT
NP3_E6
(SEQ ID NO: 177)
ILPGSDNTNY
NP3_E11
(SEQ ID NO: 178)
ISTYSGNT
NP3_F4
(SEQ ID NO: 179)
INTNTGEP
NP3_F7
(SEQ ID NO: 180)
IYPGNGDT
NP3_F8
(SEQ ID NO: 181)
IWSDGNT
NP3_F10
(SEQ ID NO: 182)
IDPSDSET
NP3_G1
(SEQ ID NO: 183)
INPSNGGT
NP3_G5
(SEQ ID NO: 184)
INTYTGEP
NP3_G6
(SEQ ID NO: 185)
ISDGGSYT
NP3_G7
(SEQ ID NO: 186)
IYPGNGDT
NP3_G8
(SEQ ID NO: 187)
KNTESGEE
NP3_G9
(SEQ ID NO: 188)
IYPGNDDT
NP3_G12
(SEQ ID NO: 189)
ILPGSGNT
NP3_H2
(SEQ ID NO: 190)
FNPSSDY
NP3_H9
(SEQ ID NO: 191)
INAETGEP
NP3_H12
(SEQ ID NO: 192)
INTYTGEP
Heavy Chain CDR3 Sequences of SEQ ID NOs: 1-48

Each of the heavy chain sequences of SEQ ID NOs: 1-48 were analyzed to identify the heavy chain CDR3 (CDRH3) sequences. SEQ ID NOs: 193-240 constitute CDRH3 sequences within SEQ ID NOs: 1-48.

NP3_A3
(SEQ ID NO: 193)
ARHYRYDEAWFAY
NP3_A4
(SEQ ID NO: 194)
ARSGYRGNFDY
NP3_A6
(SEQ ID NO: 195)
VRGYRYDEAWFVY
NP3_A7
(SEQ ID NO: 196)
ARYRIYYFDY
NP3_A8
(SEQ ID NO: 197)
TRLEGYGDSDYAMIY
NP3_A9
(SEQ ID NO: 198)
ARGDYDGGYGMDY
NP3_A10
(SEQ ID NO: 199)
ARGLRFFYAMDY
NP3_A11
(SEQ ID NO: 200)
ARSHGNYENHFDY
NP3_B1
(SEQ ID NO: 201)
ALPWDY
NP3_B2
(SEQ ID NO: 202)
ARGSTYGNPLDY
NP3_B3
(SEQ ID NO: 203)
TRKELFGYGFDY
NP3_B4
(SEQ ID NO: 204)
AFDVYYGGAMDY
NP3_B5
(SEQ ID NO: 205)
AIYYDYDDYIMDY
NP3_B8
(SEQ ID NO: 206)
TRGDDYGGRLAY
NP3_B9
(SEQ ID NO: 207)
ARRDDVYYVAGFDY
NP3_B10
(SEQ ID NO: 208)
ARGAATRY
NP3_B12
(SEQ ID NO: 209)
ARRGLYDGYYVRYFDV
NP3_C3
(SEQ ID NO: 210)
ARSHDNYDNHFDY
NP3_C4
(SEQ ID NO: 211)
ADNGYL
NP3_C5
(SEQ ID NO: 212)
ARRDDVYYVAGFDY
NP3_C6
(SEQ ID NO: 213)
ARRKFYDGNYVRYFDV
NP3_C8
(SEQ ID NO: 214)
ARAGEGYAMDY
NP3_C9
(SEQ ID NO: 215)
SRGAATGF
NP3_C10
(SEQ ID NO: 216)
ARTVYHGAMDY
NP3_C11
(SEQ ID NO: 217)
VRDKYDGWGSDFDV
NP3_D1
(SEQ ID NO: 218)
ALPWDY
NP3_D4
(SEQ ID NO: 219)
ARGLRFFYAMDY
NP3_D5
(SEQ ID NO: 220)
TRGTGDFDY
NP3_E2
(SEQ ID NO: 221)
ASGGNYGLDY
NP3_E3
(SEQ ID NO: 222)
ARHYKYDEAWSPY
NP3_E4
(SEQ ID NO: 223)
AKEDDGYLFYFDY
NP3_E5
(SEQ ID NO: 224)
ARGSTYGNPLDH
NP3_E6
(SEQ ID NO: 225)
ARRLFLRRYFDV
NP3_E11
(SEQ ID NO: 226)
TRGTGDFDY
NP3_F4
(SEQ ID NO: 227)
ARGSIYDGYYEGLYY
NP3_F7
(SEQ ID NO: 228)
ARRPLFYAMDY
NP3_F8
(SEQ ID NO: 229)
ARNSGYNDYDGYYFDV
NP3_F10
(SEQ ID NO: 230)
AREVTTATKGVFSWLAY
NP3_G1
(SEQ ID NO: 231)
TRLEGYGDYDYAMYY
NP3_G5
(SEQ ID NO: 232)
ARAPLLRSYFDY
NP3_G6
(SEQ ID NO: 233)
ARGSTYGNPLDY
NP3_G7
(SEQ ID NO: 234)
ARRPLFYAMDY
NP3_G8
(SEQ ID NO: 235)
ARSGDIYAMDY
NP3_G9
(SEQ ID NO: 236)
ARRPLFYAMDY
NP3_G12
(SEQ ID NO: 237)
ARRLFLRRYFDV
NP3_H2
(SEQ ID NO: 238)
ARAGEVYAKDY
NP3_H9
(SEQ ID NO: 239)
ARHYRYDEAWFVY
NP3_H12
(SEQ ID NO: 240)
ARTREIWGNVDFPY
Light Chain CDR1 Sequences of SEQ ID NOs: 49-96

Each of the light chain sequences of SEQ ID NOs: 49-96 were analyzed to identify the light chain CDR1 (CDRL1) sequences. SEQ ID NOs: 241-288 constitute CDRL1 sequences within SEQ ID NOs: 49-96.

NP3_A3
(SEQ ID NO: 241)
ENIYSN
NP3_A4
(SEQ ID NO: 242)
QDVSTA
NP3_A6
(SEQ ID NO: 243)
ENIYSN
NP3_A7
(SEQ ID NO: 244)
KSLLHSNGNTY
NP3_A8
(SEQ ID NO: 245)
KSVSASGYSY
NP3_A9
(SEQ ID NO: 246)
KSLLHSNGFTY
NP3_A10
(SEQ ID NO: 247)
QSVDYDGDSY
NP3_A11
(SEQ ID NO: 248)
ENIYSS
NP3_B1
(SEQ ID NO: 249)
QTLLDSDGKTY
NP3_B2
(SEQ ID NO: 250)
QGISNY
NP3_B3
(SEQ ID NO: 251)
QSVDYDGDSY
NP3_B4
(SEQ ID NO: 252)
SSVSY
NP3_B5
(SEQ ID NO: 253)
SSVNY
NP3_B8
(SEQ ID NO: 254)
QGISNY
NP3_B9
(SEQ ID NO: 255)
ENIYSN
NP3_B10
(SEQ ID NO: 256)
QSVDFDGKSY
NP3_B12
(SEQ ID NO: 257)
QSVDYDGYSY
NP3_C3
(SEQ ID NO: 258)
ENIYSN
NP3_C4
(SEQ ID NO: 259)
QSLLDSDGKTY
NP3_C5
(SEQ ID NO: 260)
ENIYSN
NP3_C6
(SEQ ID NO: 261)
QSVDYDGDSY
NP3_C8
(SEQ ID NO: 262)
QSVDYDGDSY
NP3_C9
(SEQ ID NO: 263)
QSVDYDGDSY
NP3_C10
(SEQ ID NO: 264)
SSSVSY
NP3_C11
(SEQ ID NO: 265)
SSVSSNY
NP3_D1
(SEQ ID NO: 266)
QSLLDSDGKTY
NP3_D4
(SEQ ID NO: 267)
QSVDYDGDSY
NP3_D5
(SEQ ID NO: 268)
QDVGTA
NP3_E2
(SEQ ID NO: 269)
QDVGSA
NP3_E3
(SEQ ID NO: 270)
ENIYSN
NP3_E4
(SEQ ID NO: 271)
QDISDY
NP3_E5
(SEQ ID NO: 272)
QGISNY
NP3_E6
(SEQ ID NO: 273)
QSVDYDGDSY
NP3_E11
(SEQ ID NO: 274)
QDVGSA
NP3_F4
(SEQ ID NO: 275)
ENIYSN
NP3_F7
(SEQ ID NO: 276)
QSVDYDGDSY
NP3_F8
(SEQ ID NO: 277)
SSVSSSY
NP3_F10
(SEQ ID NO: 278)
QDISDY
NP3_G1
(SEQ ID NO: 279)
KSVSASGYSY
NP3_G5
(SEQ ID NO: 280)
QDVSTA
NP3_G6
(SEQ ID NO: 281)
QGISNY
NP3_G7
(SEQ ID NO: 282)
ENIYSN
NP3_G8
(SEQ ID NO: 283)
QSVDYDGGSY
NP3_G9
(SEQ ID NO: 284)
QSVDYDGESY
NP3_G12
(SEQ ID NO: 285)
QSVDYDGDSY
NP3_H2
(SEQ ID NO: 286)
QSVDYDGDSY
NP3_H9
(SEQ ID NO: 287)
ENIYSN
NP3_H12
(SEQ ID NO: 288)
ENIYSD
Light Chain CDR2 Sequences of SEQ ID NOs: 49-96

Each of the light chain sequences of SEQ ID NOs: 49-96 were analyzed to identify the light chain CDR2 (CDRL2) sequences. SEQ ID NOs: 289-336 constitute CDRL2 sequences within SEQ ID NOs: 49-96.

NP3_A3
(SEQ ID NO: 289)
GAT
NP3_A4
(SEQ ID NO: 290)
SAS
NP3_A6
(SEQ ID NO: 291)
GAT
NP3_A7
(SEQ ID NO: 292)
RMS
NP3_A8
(SEQ ID NO: 293)
LAS
NP3_A9
(SEQ ID NO: 294)
QMS
NP3_A10
(SEQ ID NO: 295)
AAS
NP3_A11
(SEQ ID NO: 296)
TAT
NP3_B1
(SEQ ID NO: 297)
LVS
NP3_B2
(SEQ ID NO: 298)
FTS
NP3_B3
(SEQ ID NO: 299)
AAS
NP3_B4
(SEQ ID NO: 300)
DTS
NP3_B5
(SEQ ID NO: 301)
DTS
NP3_B8
(SEQ ID NO: 302)
YTS
NP3_B9
(SEQ ID NO: 303)
GAT
NP3_B10
(SEQ ID NO: 304)
VAS
NP3_B12
(SEQ ID NO: 305)
AAS
NP3_C3
(SEQ ID NO: 306)
VAT
NP3_C4
(SEQ ID NO: 307)
LVS
NP3_C5
(SEQ ID NO: 308)
GAT
NP3_C6
(SEQ ID NO: 309)
AAS
NP3_C8
(SEQ ID NO: 310)
AAS
NP3_C9
(SEQ ID NO: 311)
VAS
NP3_C10
(SEQ ID NO: 312)
DTS
NP3_C11
(SEQ ID NO: 313)
STS
NP3_D1
(SEQ ID NO: 314)
LVS
NP3_D4
(SEQ ID NO: 315)
AAS
NP3_D5
(SEQ ID NO: 316)
WAS
NP3_E2
(SEQ ID NO: 317)
WTS
NP3_E3
(SEQ ID NO: 318)
AAT
NP3_E4
(SEQ ID NO: 319)
YTS
NP3_E5
(SEQ ID NO: 320)
YTS
NP3_E6
(SEQ ID NO: 321)
AAS
NP3_E11
(SEQ ID NO: 322)
WTS
NP3_F4
(SEQ ID NO: 323)
AAT
NP3_F7
(SEQ ID NO: 324)
AAS
NP3_F8
(SEQ ID NO: 325)
RTS
NP3_F10
(SEQ ID NO: 326)
YTS
NP3_G1
(SEQ ID NO: 327)
LAS
NP3_G5
(SEQ ID NO: 328)
SAS
NP3_G6
(SEQ ID NO: 329)
FTS
NP3_G7
(SEQ ID NO: 330)
VAT
NP3_G8
(SEQ ID NO: 331)
AAS
NP3_G9
(SEQ ID NO: 332)
AAS
NP3_G12
(SEQ ID NO: 333)
AAS
NP3_H2
(SEQ ID NO: 334)
AAS
NP3_H9
(SEQ ID NO: 335)
AAT
NP3_H12
(SEQ ID NO: 336)
AAT
Light Chain CDR3 Sequences of SEQ ID NOs: 49-96

Each of the light chain sequences of SEQ ID NOs: 49-96 were analyzed to identify the light CDR3 (CDRL3) sequences. SEQ ID NOs: 337-384 constitute CDRL3 sequences within SEQ ID NOs: 49-96.

NP3_A3
(SEQ ID NO: 337)
QHFWGTPWT
NP3_A4
(SEQ ID NO: 338)
QQHYSNPLT
NP3_A6
(SEQ ID NO: 339)
QHFWGTPWT
NP3_A7
(SEQ ID NO: 340)
MQHLEYPLT
NP3_A8
(SEQ ID NO: 341)
QHSRELPLT
NP3_A9
(SEQ ID NO: 342)
AQNLELP
NP3_A10
(SEQ ID NO: 343)
QQSIEDPYT
NP3_A11
(SEQ ID NO: 344)
QHFWGFPWT
NP3_B1
(SEQ ID NO: 345)
WQGTHFPQT
NP3_B2
(SEQ ID NO: 346)
QQYSKLPFT
NP3_B3
(SEQ ID NO: 347)
QQSNEDPYT
NP3_B4
(SEQ ID NO: 348)
FQGSGYPLT
NP3_B5
(SEQ ID NO: 349)
HQRSSFPFT
NP3_B8
(SEQ ID NO: 350)
QQYNKLPYT
NP3_B9
(SEQ ID NO: 351)
QLFWGTPWT
NP3_B10
(SEQ ID NO: 352)
QQTNEDPLT
NP3_B12
(SEQ ID NO: 353)
QQSNADPFT
NP3_C3
(SEQ ID NO: 354)
QHFWGTPWT
NP3_C4
(SEQ ID NO: 355)
WQGTHFPRT
NP3_C5
(SEQ ID NO: 356)
QHFWGTPWT
NP3_C6
(SEQ ID NO: 357)
QQSNEDPWT
NP3_C8
(SEQ ID NO: 358)
QQSIGDPWT
NP3_C9
(SEQ ID NO: 359)
QQTNEDPLT
NP3_C10
(SEQ ID NO: 360)
QQWSGYPYT
NP3_C11
(SEQ ID NO: 361)
HQFHRSPRT
NP3_D1
(SEQ ID NO: 362)
WQGTHFPQT
NP3_D4
(SEQ ID NO: 363)
QQSIEDPYT
NP3_D5
(SEQ ID NO: 364)
HQYSSYPYT
NP3_E2
(SEQ ID NO: 365)
QQYSRYPLT
NP3_E3
(SEQ ID NO: 366)
QHFWGTPWT
NP3_E4
(SEQ ID NO: 367)
QQYSKLPYT
NP3_E5
(SEQ ID NO: 368)
QQYSKLPFT
NP3_E6
(SEQ ID NO: 369)
QQSNEDPFT
NP3_E11
(SEQ ID NO: 370)
HQYSSYPYT
NP3_F4
(SEQ ID NO: 371)
QHFWGFPRT
NP3_F7
(SEQ ID NO: 372)
QQSNEDPWT
NP3_F8
(SEQ ID NO: 373)
QQWSGYPFT
NP3_F10
(SEQ ID NO: 374)
QQYSKLPWT
NP3_G1
(SEQ ID NO: 375)
QHRREIPLT
NP3_G5
(SEQ ID NO: 376)
QQYYSTPFT
NP3_G6
(SEQ ID NO: 377)
QQYSKLPFT
NP3_G7
(SEQ ID NO: 378)
CQHFWGTPWT
NP3_G8
(SEQ ID NO: 379)
QQSNEDPWT
NP3_G9
(SEQ ID NO: 380)
QQSYEDPWT
NP3_G12
(SEQ ID NO: 381)
QQSNEDPFT
NP3_H2
(SEQ ID NO: 382)
QQSNEDPWT
NP3_H9
(SEQ ID NO: 383)
QHFWGTPWT
NP3_H12
(SEQ ID NO: 384)
QHFWGTPWT

### Example 3. Consensus Sequences of Heavy and Light Chain Sequences

The antibodies were analyzed to identify consensus sequences of the heavy chain and light chain sequences.

### Consensus Sequence of the Heavy Chain Sequences of SEQ ID NOs: 1-48

The heavy chains of SEQ ID NOs: 1-48 were analyzed for variability across each amino acid position. SEQ ID NO: 385 represents a consensus sequence based on this analysis.

(SEQ ID NO: 385)
X1X2X3X4X5X6X7X8X9X10X11X12X13X14X15X16X17X18X19
X20X21X22X23X24X25X26X27X28X29X30X31X32X33X34X35
X36X37X38X39X40X41X42X43X44X45X46X47X48X49X50X51
X52X53X54X55X56X57X58X59X60X61X62X63X64X65X66X67
X68X69X70X71X72X73X74X75X76X77X78X79X80X81X82X83
X8X85X86X87X88X89X90X91X92X93X94X95X96X97X98X99
X100X101X102X103X104X105X106X107X108X109X110X111
X112X113X114X115X116X117X118X119X120X121X122X123
X124X125X126X127

wherein,
X1=D, Q, or E;
X2=V, I, or A;
X3=Q or T;
X4=L;
X5=Q, V, or K;
X6=E or Q;
X7=S;
X8=G;
X9=P, A, T, G, or H;
X10=E, D, V, G, or S;
X11=L, P, V, or I;
X12=K, R, V, A, M, or L;
X13=K, T, R, E, Q, or A;
X14=P;
X15=G or S;
X16=E, A, S, V, T, G, or Q;
X17=T, P, or S;
X18=V or L;
X19=K, R, T, or S;
X20=I, L, M, V, or F;
X21=S, A, P, Y, or T;
X22=C;
X23=K, T, A, L, or S;
X24=A, V, T, G, or F;
X25=S, F, or T;
X26=G;
X27=Y, F, S, or D;
X28=T, I, M, N, S, or A;
X29=F, I, L, or V;
X30=T, K, or S;
X31=D, N, S, or T;
X32=S or is absent;
X33=G or is absent;
X34=Y, C, T, D, M, or L;
X35=A, S, P, G, Y, W, T, V, or N;
X36=I, M, L, or V, or is absent;
X37=H, N, Y, E, D, S, or G;
X38=W;
X39=V or I;
X40=K, R, or Q;
X41=Q, L, E, or K;
X42=A, V, R, S, T, P, or F;
X43=P, T, H, or S;
X44=G, E, A, T, or V;
X45=K, Q, R, H, or N;
X46=G, D, S, R, or K;
X47=L or F;
X48=K, E, or A;
X49=W or F;
X50=M, V, L, or I;
X51=G or A;
X52=W, R, E, Y, A, Q, D, V, T, or H;
X53=I, K, F, or M;
X54=N, D, Y, F, L, S, R, or W;
X55=T, A, P, or L, or is absent;
X56=E, Y, N, A, S, G, D, or W;
X57=T, S, N, D, Y, or G;
X58=G, D, N, or S;
X59=E, K, D, G, A, Y, N, S, or T;
X60=Y or is absent;
X61=A or is absent;
X62=P, E, T, F, N, or K;
X63=K, T, I, S, N, H, Y, or M;
X64=Y, F, or H;
X65=A, E, G, D, N, V, I, P, H, or S;
X66=E, D, G, P, H, Q, or S;
X67=D, K, N, R, I, H, S, A, or V;
X68=F, V, or L;
X69=K, E, Q, R, M, or I;
X70=G, R, D, or S;
X71=R, K, or Q;
X72=F, A, V, L, or I;
X73=A, V, D, T, I, K, S, or N;
X74=F, V, L, M, I, or S;
X75=S, I, T, A, or N;
X76=L, A, V, R, or K;
X77=E, D, or V;
X78=T, I, K, R, N, D, or Y;
X79=S, F, or A;
X80=V, A, G, S, K, or R;
X81=S, G, N, or T;
X82=T, A, N, R, Q, or H;
X83=A, V, L, or Y;
X84=Y, F, or N;
X85=L, S, M, or I;
X86=Q, R, E, H, or K;
X87=S, I, L, M, or G;
X88=N, T, S, K, G, R, I, A, or H;
X89=N, S, G, or R;
X90=L or V;
X91=K, N, T, R, D, or Q;
X92=N, S, G, or T;
X93=E, A, or D;
X94=D;
X95=T, S, or M;
X96=A, T, or G;
X97=T, S, A, V, I, or M;
X98=Y or F;
X99=F, Y, or S;
X100=C;
X101=A, V, S, or T;
X102=R, I, S, L, D, F, or K;
X103=Y, L, E, G, or D, or is absent;
X104=H, G, S, T, R, A, E, Y, V, K, D, or N, or is absent;
X105=Y, H, R, D, S, P, A, G, T, E, or K, or is absent;
X106=I, L, Y, T, F, G, or V, or is absent;
X107=Y, L, G, A, P, or N, or is absent;
X108=G, D, E, R, T, H, L, or N, or is absent;
X109=R, K, N, I, V, G, S, Y, E, D, or F, or is absent;
X110=Y, W, D, G, V, F, N, or T, or is absent;
X111=D, E, G, Y, F, V, L, or W, or is absent;
X112=E, N, V, D, -, I, A, F, G, R
X113=A, V, Y, S, R, F, N, or G, or is absent;
X114=W, H, D, G, A, Y, P, R, or N, or is absent;
X115=F, S, L, T, M, K, W, or G, or is absent;
X116=A, V, P, D, Y, R, G, or I, or is absent;
X117=Y, F, V, L, or H, or is absent;
X118=W;
X119=G;
X120=R, Q, or A;
X121=G;
X122=T;
X123=L, T, or S;
X124=V or L;
X125=T;
X126=V;
X127=S or F; and
X128=S or A;

### Consensus Sequence of the Light Chain Sequences of SEQ ID NOs: 49-96

The light chains of SEQ ID NOs: 49-96 were analyzed for variability across each amino acid position. SEQ ID NO: 386 represents a consensus sequence based on this analysis.

(SEQ ID NO: 386)
X1X2X3X4X5X6X7X8X9X10X11X12X13X14X15X16X17X18X19
X20X21X22X23X24X25X26X27X28X29X30X31X32X33X34X35
X36X37X38X39X40X41X42X43X44X45X46X47X48X49X50X51
X52X53X54X55X56X57X58X59X60X61X62X63X64X65X66X67
X68X69X70X71X72X73X74X75X76X77X78X79X80X81X82X83
X84X85X86X87X88X89X90X91X92X93X94X95X96X97X98X99
X100X101X102X103X104X105X106X107X108X109X110X111
X112

wherein,
X1=D or E;
X2=I, V, or N;
X3=V, Q, L, or K;
X4=M, L, or I;
X5=T or N;
X6=Q;
X7=S, A, or T;
X8=P, A, H, or T;
X9=A, S, P, K, L, or G;
X10=S, F, I, L, or T;
X11=L, M, V, or N;
X12=S, A, P, V, or T;
X13=V, T, A, or L;
X14=S or T;
X15=V, L, P, or I;
X16=G;
X17=E, D, G, Q, or T;
X18=T, R, K, S, or P;
X19=V, I, or A;
X20=T or S;
X21=I, M, or L;
X22=T, N, or S;
X23=C;
X24=R, K, S, or T;
X25=S, A, or T;
X26=S or N;
X27=E, Q, S, or K;
X28=N, D, G, S, or T;
X29=I, V, or L, or is absent;
X30=L or V, or is absent;
X31=H, D, S, or is absent;
X32=S, A, Y, or F;
X33=N, D, or S, or is absent;
X34=S, G, or N, or is absent;
X35=Y, S, N, F, K, D, G, or E, or is absent;
X36=S, T, N, or D, or is absent;
X37=N, S, D, A, or Y;
X38=L, V, M, or I;
X39=A, V, N, H, Y, or S;
X40=W;
X41=F, Y, or L;
X42=Q, L, or F;
X43=Q, H, or E;
X44=K, R, or I;
X45=Q, E, P, or S;
X46=G, D, or S;
X47=K, Q, G, T, S, or A;
X48=S, T, or P;
X49=P, L, V, or I;
X50=Q, K, or R;
X51=L, P, R, or V;
X52=L or W;
X53=V or I;
X54=Y, N, S, or H;
X55=G, V, A, T, S, W, F, Y, D, R, Q, or L;
X56=A, T, M, or V;
X57=T or S;
X58=D, N, H, Y, T, S, I, E, or K;
X59=L, R, or P;
X60=A, Y, H, D, E, P, or Q;
X61=D, T, or S;
X62=G or N;
X63=V, F, A, or I;
X64=P;
X65=S, D, G, V, A, or P;
X66=R, H, or G;
X67=F;
X68=S, T, or G;
X69=G or S;
X70=S, N, or G;
X71=G or R;
X72=S or A;
X73=G or D;
X74=T, S, N, or A;
X75=Q, H, D, S, or A;
X76=Y, F, or I;
X77=S or T;
X78=L or F;
X79=K, R, T, I, N, or S;
X80=I;
X81=N, S, or H;
X82=S, N, R, or P;
X83=L, V, or M;
X84=Q or E;
X85=S, A, P, E, or D;
X86=E;
X87=D;
X88=F, L, I, V, A, or D;
X89=G, A, or V;
X90=T, S, N, V, I, or D;
X91=Y or F;
X92=Y, F, or S;
X93=C;
X94=Q, H, F, M, A, or W;
X95=H, L, or Q;
X96=F, H, Y, G, W, R, N, S, or T;
X97=W, Y, S, N, H, L, T, R, or I;
X98=G, S, R, K, E, H, or A;
X99=T, F, N, Y, L, S, I, or D;
X100=P;
X101=W, R, L, F, Y, or Q;
X102=T;
X103=F;
X104=G;
X105=G, A, S, or T;
X106=G;
X107=T;
X108=K;
X109=L;
X110=E;
X111=I or L; and
X112=K.

### Example 4. Consensus Sequences of the CDR Sequences

The CDR sequences were analyzed further to identify consensus sequences of the heavy chain and light chain CDR sequences.

Consensus sequence of CDRH1 sequences (SEQ ID NOs: 97-144)

The heavy chain CDR1 (CDRH1) sequences of SEQ ID NOs: 97-144 were analyzed for variability across each amino acid position. SEQ ID NO: 387 represents a consensus sequence based on this analysis.

(SEQ ID NO: 387)
X1X2X3X4X5X6X7X8X9X10

wherein,
X1=G;
X2=Y, F, S, or D;
X3=T, I, M, S, A, or N;
X4=F, L, I, or V;
X5=T, S, or K;
X6=D, T, S, or N;
X7=Y, C, D, S, T, or G;
X8=A, S, N, P, V, T, G, Y, or W, or is absent;
X9=M or L, or is absent; and
X10=G or is absent.
   Consensus sequence of CDRH2 sequences (SEQ ID NOs: 145-192)

The heavy chain CDR2 (CDRH2) sequences of SEQ ID NOs: 145-192 were analyzed for variability across each amino acid position. SEQ ID NO: 388 represents a consensus sequence based on this analysis.

(SEQ ID NO: 388)
X1X2X3X4X5X6X7X8X9X10X11X12

wherein,
X1=I, K, or F, or is absent;
X2=N, I, M, S, D, Y, F, L, or R;
X3=T, A, S, R, P, D, L, or W;
X4=E, N, Y, A, S, D, G, or W;
X5=T, S, N, D, Y, or G;
X6=G, D, N, or S;
X7=E, K, S, N, T, D, G, A, or Y, or is absent;
X8=P, E, T, F, N, or K, or is absent;
X9=N or is absent;
X10=Y or is absent;
X11=A or is absent; and
X12=T or is absent.

### Consensus Sequence of CDRH3 Sequences (SEQ ID NOs: 193-240)

The heavy chain CDR3 (CDRH3) sequences of SEQ ID NOs: 193-240 were analyzed for variability across each amino acid position. SEQ ID NO: 389 represents a consensus sequence based on this analysis.

(SEQ ID NO: 389)
X1X2X3X4X5X6X7X8X9X10X11X12X13X14X15X16X17X18

wherein,
X1=A, V, T, or S, or is absent;
X2=R, I, K, S, or F, or is absent;
X3=H, G, Y, R, N, D, A, S, E, T, L, or K, or is absent;
X4=Y, R, D, S, G, K, L, P, H, E, V, or A, or is absent;
X5=R, K, D, I, L, F, G, E, T, or A, or is absent;
X6=Y, L, V, I, F, T, or N, or is absent;
X7=D, N, R, G, Y, W, or A, or is absent;
X8=E, D, G, Y, T, or F, or is absent;
X9=G, Y, N, W, R, E, D, V, L, or K, or is absent;
X10=Y, D, G, N, A, F, V, L, R, or S, or is absent;
X11=E, N, H, G, Y, D, V, R, P, or T, or is absent;
X12=G, V, F, S, Y, L, I, or H, or is absent;
X13=A, Y, M, L, R, S, D, P, F, or G, or is absent;
X14=W, I, Y, D, N, G, or L, or is absent;
X15=A, P, or R, or is absent;
X16=F, S, M, Y, L, K, or W, or is absent;
X17=A, V, P, D, I, or Y, or is absent; and
X18=Y, V, or H, or is absent.

### Consensus Sequence of CDRL1 Sequences (SEQ ID NOs: 241-288)

The light chain CDR1 (CDRL1) sequences of SEQ ID NOs: 241-288 were analyzed for variability across each amino acid position. SEQ ID NO: 390 represents a consensus sequence based on this analysis.

(SEQ ID NO: 390)
X1X2X3X4X5X6X7X8X9X10X11

wherein,
X1=E, Q, or K, or is absent;
X2=N, D, G, S, or T, or is absent;
X3=I, V, or L, or is absent;
X4=Y, S, G, L, or V, or is absent;
X5=S, T, N, D, or H, or is absent;
X6=N, S, D, A, Y, or F, or is absent;
X7=N, D, S, or V, or is absent;
X8=G or S, or is absent;
X9=N, F, K, Y, D, E, G, V, or S, or is absent;
X10=T, S, or N, or is absent; and
X11=Y or is absent.

### Consensus Sequence of CDRL2 Sequences (SEQ ID NOs: 289-336)

The light chain CDR2 (CDRL2) sequences of SEQ ID NOs: 289-336 were analyzed for variability across each amino acid position. SEQ ID NO: 391 represents a consensus sequence based on this analysis.

(SEQ ID NO: 391)
X1X2X3

wherein,
X1=G, A, T, V, S, L, R, Q, F, Y, W, or D;
X2=A, M, V, or T; and
X3=T or S.

### Consensus Sequence of CDRL3 Sequences (SEQ ID NOs: 337-384)

The light chain CDR3 (CDRL3) sequences of SEQ ID NOs: 337-384 were analyzed for variability across each amino acid position. SEQ ID NO: 392 represents a consensus sequence based on this analysis.

(SEQ ID NO: 392)
X1X2X3X4X5X6X7X8X9X10

wherein,
X1=C or M, or is absent;
X2=Q, H, W, F, or A, or is absent;
X3=H, L, or Q;
X4=F, S, R, H, Y, W, T, G, or N, or is absent;
X5=W, L, R, Y, S, N, I, H, or T;
X6=G, E, S, K, R, A, or H;
X7=T, F, Y, L, I, N, D, or S;
X8=P;
X9=W, R, L, F, Y, or Q, or is absent; and
X10=T or is absent.

### Example 5. Nucleotide Sequences of the Heavy and Light Chain Sequences

Nucleotide sequences corresponding to the Heavy Chain sequences described in Example 1 are provided below.

### Heavy Chain Nucleotide Sequences

NP3_A3

GATGTGCAGCTGCAGGAGTCTGGACCTGAGCTGAAGAAGCCTGGAGAGACAGTCAA
GATCTCCTGCAAGGCTTCTGGTTATACCTTCACAGACTATGCAATTCACTGGGTGAA
GCAGGCTCCAGGAAAGGGTTTAAAGTGGATGGGCTGGATAAACACTGAGACTGGCG
AGCCAAAATATGCAGAGGACTTCAAGGGACGGTTTGCCTTCTCTTTGGAAACCTCTG
TCAGCACTGCCTATTTGCAGAGCAACAACCTCAAAAACGAGGACACGGCTACATAT
TTCTGTGCTAGACACTATAGGTACGACGAGGCCTGGTTTGCTTACTGGGGCCGAGGG
ACTCTGGTCACAGTCTCCTCA (SEQ ID NO: 393)
NP3_A4
CAGGTTACTCTGAAAGAGTCTGGCCCCGGGATATTGAAGCCCTCACAGACCCTCAGT
CTGACTTGTTCTTTCTCTGGGTTTTCACTGAGCACTTCTGGTATGGGTGTAGCTGGAT
TCGTCAGCCTTCAGGGAAGGGTCTGGAGTGGCTGGCACACATTTGGTGGGTGATGAT
AAGTACCATAACCCATCCCTGAAGAGCCAGCTCACAATCTCCAAGGATACCTCCAG
AAACCAGGTATTCCTCAAGATCACCAGTGTGGACACTGCAGATACTGCCACTTACTA
CTGTGCTCGAAGCGGATATAGGGGAAACTTTGACTACTGGGGCCAAGGCA
CCACGGTCACCGTCTCCTCA (SEQ ID NO: 394)
NP3_A6
CAGATCCAGTTGGTGCAGTCTGGACCTGAGTTGAAGAAGCCTGGAGAGACAGTCAA
GATCGCCTGTAAGGCTTCTGGTTATACCTTCACAGACTATTCAATGAACTGGGTGAA
GCAGGCTCCAGGAAAGGGTTTAAAGTGGATGGGCTGGATAAACACTGAGATGGCGA
GCCAACATATGAAGATGACTTCAAGGGACGGTTTGCCTTCTCTTTGGAAACCTCTGC
CAGCACTGCCTATTTGCAGATCACCAACCTCAAAAATGAGGACACGGCTATATTTCT
GTGTTAGAGGGTATAGGTACGACGAGGCCTGGTTTGTTTACTGGGGCCAAGGGACTC
TGGTCACTGTCTCTGCA (SEQ ID NO: 395)
NP3_A7
CAGATCCAGTTGGTGCAGTCTGGGGCAGAGCTTGTGAAGCCAGGGGCCTCAGTCAA
GTTGTCCTGCACAGCTTCTGGCTTCAACATTAAAGACACCTATATCCACTGGTGAGA
CAGAGGCCTGAACAGGGCCTGGAGTGGATTGGAAGGATTGATCCTGCGAATGGTGA
TACTAAATATGACCCGAAGTTCCAGGACAAGGCCACTTTAATAGCAGACAATCCTCC
AACACAGCCTACCTGCAGCTCGGCAGCCTGACATCTGAGGACACTGCCGTCTATTAC
TGTGCTAGATATCGTATTTACTACTTTGACTACTGGGGCCAAGGCACCACTCTCACA
GTCTCCTCA (SEQ ID NO: 396)
NP3_A8
CAGGTGCAGCTGCAGCAGTCTGGGGCTGAACTGGTGAAGCCTGGGGCTTCAGTGAA
GTTGTCCTGCAAGGCTTCAGGCTACACCTTCACCAGCTACTATATGTACTGGGTGAA
ACAGAGGCCTGGACAAGGCCTTGAGTGGATTGGAGAGATTAATCCTAGCAATGGTG
GTTTTAGCTTCAATGAGAAGTTCAAGAGTAAGGCCACACTGACTGTAGACAAGTCCT
CCAGCACAGCATACATGCAACTCACCAGCCTGACATCTGAGGACTCTGCGGTCTATT
ACTGTACAAGATTAGAGGGGTATGGTGACTCCGACTATGCTATGATCTACTGGGGTC
AAGGAACCACGGTCACCGTCTCCTCA (SEQ ID NO: 397)
NP3_A9
GAGGTGCAGCTTGTTGAGTCTGGAGCTGAGCTGGTGAAGCCTGGGGCTTCAGTGAA
GATATCCTGCAAGGCTTCTGGCTACACATTCACTGACTACAACATGGACTGGGTGAA
GCAGAGCCATGGAAAGAGCCTTGAGTGGATTGGAGATATTAATCCTGACTATGGTA
GTACTAGTTACAACCAGAAGTTCAGGGGAAAGGCCACTTTGACTGTAGACAAGTCC
TCCAGCACAGCCAACATGGAGCTCCGCAGCCTGACATCTGAGGACACTGCAGTCTA
TTACTGTGCAAGGGGGGATTACGACGGGGGCTATGGTATGGACTACTGGGGTCAAG
GAACCTCAGTCACCGTCTCCTCA (SEQ ID NO: 398)
NP3_A10
CAGGTGCAGCTGCAGCAGTCTGGACCTAGCCTCGTAAAACCGTCTCAGACTCTGTCC
TTCACCTGTTCTGTCACTGGCGACTCCGTCACCAGTGGTTACTGGAACTGGATCCGG
AAATTCCCAGGGAATAAACTTGAGTTCATGGGGTACATAAGTTACAGTGGTAGCACT
TACTACAGTCCATCTCTCAAAAGTCGAATCTCCATCATTCGCGACACATCCAAGAAC
CGGTACTACCTGCAGTTGAACTCTGTGACTACTGAGGACACAGCCACATATTTCTGT
GCAAGGGGGCTACGGTTTTTCTATGCCATGGATTACTGGGGTCAAGGAACCACTCTC
ACAGTCTCCTCA (SEQ ID NO: 399)
NP3_A11
GAGGTTCAGCTGCAGGAGTCTGGACCTGAGCTGAAGAAGCCTGGAGAGACAGTCAA
GATCTCCTGCAAGGTTTCTGGGTATACCTTCACAAACTATGGAATGAACTGGGTGAA
GCAGGCTCCAGGCAAGGGTTTAAAGTGGATGGGCTGGATAAACACCTACACTGGAG
AGCCAAAATATGGTGATGACTTCAAGGGACGGTTTGCCCTCTCTTTGGAGACCTCTG
CCGGTACTGCCTATTTGCGGATTAATAATCTCAAAAATGAGGACACGGCTACATATT
TCTGTGCAAGATCCCATGGTAACTACGAGAACCACTTTGACTACTGGGGCCAAGGCA
CCACTCTCACCGTCTCCTCA (SEQ ID NO: 400)
NP3_B1
CAGGTGCAGCTGCAGCAGTCTGGACCTGAGCTGGTGAAGCCTGGGACTTCAGTGAC
GGTATACTGCAAGGCTTCTGGTTATACATTCACTACCTACAACATTTACTGGGTGAA
GCAGAGCCATGGAAAGAGCCTTGAGTGGATTGGATATATTGATCCTTACAATGGTG
GTACTAGCTACAACCAGAAGTTCAAGGGCAAGGCCACATTGACTGTTGACAAGTCC
TCCAGCGCAGCCTACATGCATCTCAACAGCCTGACATCTGAGGACTCTGCAGTCTAT
TACTGTGCCCTCCCATGGGACTACTGGGGCCAAGGCACCACGGTCACCGTCTC
CTCA(SEQ ID NO: 401)
NP3_B2
CAGATCCAGTTGGTGCAGTCTGGGGGGGGCTTAGTGAAGCCTGGAGGGTCCCTGAA
ACTCTCCTGTGCAGCCTCTGGATTCACTTTCAGTGACTATTACATGTCTTGGGTTCGC
CAGACTCCGGAAAAGCGGCTGGAGTGGGTCGCAACCATTAGTGATGGTGGTAGTTA
CACCTACTATCCAGACAGTGTGAAGGGGCGATTCACCATCTCCAGAGACAATGCCA
AGAACAACCTGTACCTGCACATGAGCAGTCTGAAGTCTGAGGACACAGCCATGTAT
TACTGTGCAAGAGGGTCTACCTATGGTAACCCTCTTGACTACTGGGGCCAAGGCACC
ACTCTCACAGTCTCCTCA (SEQ ID NO: 402)
NP3_B3
GAGGTTCAGCTGCAGCAGTCTGGGACTGTGCTGGCAAGGCCTGGGGCTTCCGTGAA
GATGTCCTGCAAGGCTTCTGGCTCCAGCTTTACCACCTACTGGATGCACTGGGTAAA
ACAGAGGCCTGGACAGGGTCTAGAATGGATTGGTGCTATTTATCCTGGAAATAGTG
ATACTAGCTACAACCAAAAGTTCAAGGGCAAGGCCAAACTGACTGCAGTCACATCC
GCCAGCACTGCCTACATGGAGCTCAGCAGCCTGACAAATGAGGACTCTGCGGTCTA
TTACTGTACAAGAAAGGAATTATTTGGGTACGGGTTTGACTACTGGGGCCAAGGCAC
CACTCTCACAGTCTCCTCA (SEQ ID NO: 403)
NP3_B4
GAGGTGCAGCTTGTTGAGTCTGGAGGAGGCTTGGTGCAACCTGGAGGATCCGTGAA
ACTCTCCTGTTTAGCCTCTGGATTTACCTTCAGTGACTACTACATGTCTTGGGTCCGC
CAGTCTCCAGAGAAGGGGCTTGAGTGGGTTGCTGAAATTAGATTGGAATCTGATAAT
TATGCAACACATTATGCGGAGTCTGTGAAAGGGAAGTTCACCATCTCAAGAGATGA
TTCCAAAAGTCGTCTCTATCTGAAAATGAACAGCTTAAGGGGTGAAGACACTGGAA
TTTATTACTGTGCGTTTGATGTTTACTACGGGGGGGCTATGGACTACTGGGGTCAAG
GAACCACGGTCACCGTCTCCTCA (SEQ ID NO: 404)
NP3_B5
CAGGTGCAGCTGCAGCAGTCTGGGGCTGAGCTGGTGAGGCCTGGGGCTTCAGTGAA
GCTGTCCTGCAAGGCTTCTGGCTACACGTTCACCAGCTACTGGATGAACTGGGTTAA
ACAGAGGCCTGAACAAGGCCTTGCGTGGATTGGAAGGATTGATCCTTACGATAATG
CAACTCACTACAATCACAAGTTCAAGGACAAGGCCATATTGACTGTAGACAAATCCT
CCAGCACAGCCTACATGGAACTCAGAGGCCTGACATCTGAGGACTCTGCGGTCTATT
ACTGTGCAATCTACTATGATTACGACGACTATATTATGGACTACTGGGGTCAAGGAA
CCTCAGTCACCGTCTCCTCA (SEQ ID NO: 405)
NP3_B8
GATGTGCAGCTGCAGGAGTCTGGGGGAGGCTTAGTGAAGCCTGGAGGGTCCCTGAA
ACTCTCCTGTGCAGCCTCTGGATTCACTTTCAGTGACTATTACATGTATTGGGTTCGC
CAGACTCCGGAAAGGAGGCTGGAGTGGGTCGCAACCATTAGTGATGGTGGTAGTTA
CACCTACTATCCAGACAGTGTGAAGGGGCGATTCACCAGCTCCAGAGACAATGCCA
AGAACAACCTGTACCTGCAAATGAGCAGTCTGAAGTCTGAGGACACAGCCATGTAT
TACTGTACAAGAGGGGATGACTACGGTGGTAGGCTTGCTTACTGGGGCCAAGGGAC
TCTGGTCACTGTCTCTGCA (SEQ ID NO: 406)
NP3_B9
GAGGTTCAGCTGCAGCAGTCTGGACCTGAGTTGAAGAAGCCTGGAGAGACAGTCAA
GATCTCCTGCAAGACTTCTGGATATACCTTCACAAACTGTGGCATGCACTGGGTGAA
GCAGGCTCCAGGAAAGGGTTTAAAGTGGATGGGCTGGATAAACACCTACACTGGAG
AGCCAACATATGCTGATGACTTCAAGGGACGGTTTGCCTTCTCTTTGGAAACCTCTG
TCAACACTGCCTATTTGCAGATCAACAACCTCAAAAGTGAGGACACGGCTTCATATT
TCTGTGCAAGAAGGGATGATGTTTACTACGTCGCCGGGTTTGACTACTGGGGCCAAG
GCACCACTCTCACAGTCTCCTCA (SEQ ID NO: 407)
NP3_B10
CAGATCCAGTTGGTGCAGTCTGGACCTGAACTGAAGAAGCCTGGAGAGACAGTCAA
GATCTCCTGCAAGGCTTCTGGTTATACCTTCACAGACTATTCAATGCACTGGGTGAA
GCAGGCTCCAGGAAAGGATTTAAAGTGGGTGGGCTGGATAAACACTGAGACTGGTG
AGCCAACATATGCAGATGACTTCAAGGGACGGTTTGCCTTCTCTTTGGAGACCTCTG
CCACCACTGCCTATTTGCAAATCAACAACCTCAAAAATGAGGACACGGCTACATATT
TCTGTGCTAGAGGGGCGGCTACGAGATACTGGGGCCAAGGCACCACTCTCACAGTC
TCCTCA (SEQ ID NO: 408)
NP3_B12
GAGGTTCAGCTGCAGCAGTCTGGAGCTGAGCTGATGAAGCCTGGGGCCTAGTGAAG
ATATCCTGCAAGGCTACTGGCTACACATTCAGTAGCTACTGGATAGAGTGGGTAAAG
CAGAGGCCTGGACATGGCCTTGAGTGGATTGGAGAGTTTTTTCCTGGTAGTGGTAAT
ACTAACTACAATGAGAAGTTCAAGGGCAAGGCCACATTCACTGCAGATACATCCTC
CAACACAGCCTACATGCAACTCAGCAGCCTGACATCTGAGGACTCTGCCGTCTATTA
CTGTGCGAGAAGGGGCCTCTATGATGGTTACTACGTTCGGTACTTCGATGTCTGGGG
CGCAGGGACCACGGTCACCGTCTCCTCA (SEQ ID NO: 409)
NP3_C3
CAGATCCAGTTGGTGCAGTCTGGACCTGAGCTGAGGAAGCCTGGAGAGACAGTCAA
GATCTCCTGCAAGGCTTCTGGGTATATCTTCACAAACTATGGAATGAACTGGGTGAA
GCAGGCTCCAGGAAAGGGTTTAAAGTGGATGGGCTGGATAAACACTTACACTGGAG
AGCCAACATATGCTGGTGACTTCAAGGGACGGTTTGCCTTCTCTTTGGAAACCTCTG
CCAGCACTGCCTATTTGCAGATCAACAACCTCAAAAATGAGGACTCGGCTACATATT
TCTGTGCAAGATCCCATGATAACTACGATAACCACTTTGACTACTGGGGCCAAGGCA
CCACTCTCACAGTCTCCTCA (SEQ ID NO: 410)
NP3_C4
CAGATCCAGTTGGTGCAGTCTGGACCTGAGCTGGTGAAGCCTGGGGCTTCAGTGAA
GATATCCTGCAAGGCTTCTGGTTACTCATTCACTAACTACTACATGCACTGGGTGAG
GCAAAGCCATGTAAAGAGCCTTGAGTGGATTGGACGTATTAATCCTTACAGTGGTGC
TACTAGCTACAACCAGCATTTCAAGGACAAGGCCAGCTTGACTGTCGATAAGTCCTC
CAGCACAGCCTACATGGAGCTCCACAGCCTGACATCTGAGGACTCTGCAGTCTATTA
CTGTGCAGACAATGGTTACCTTTGGGGTCAAGGAACCTCAGTCACCGTCTCCTCA
(SEQ ID NO: 411)
NP3_C5
CAGATCCAGTTGGTGCAGTCTGGACCTGAGCTGAAGACGCCTGGAGAGACAGTCAA
GATCTCCTGCAAGGCTTCTGGGTATACCTTCACAAACTGTGGAATGCACTGGGTGAA
GCAGGCTCCAGGAAAGGGTTTAAAGTGGATGGGCTGGATAAACACCTACACTGGGG
AGCCAATATATGCTGATGACTTCAAGAGACGGTTTGCCTTCTCTTTGGAAACCTCTG
CCAGCACTGCCTATTTGCAGATCAACAACCTCAATAATGAGGACACGGCTTCATATT
TCTGTGCAAGAAGGGATGATGTTTACTACGTCGCCGGGTTTGACTACTGGGGCCAAG
GCACCACGGTCACCGTCTTCTCA (SEQ ID NO: 412)
NP3_C6
CAGGTGCAGCTGCAGCAGTCTGGGCCTGAGCTGGTGAAGCCTGGGGCTTCAGTGAA
GATGTCCTGCAAGGCTTCTGGATACACATTCACTGACTATGTTATAAACTGGGTGAA
GGAGAGAACTGGACAGGGCCTTGAGTGGATTGGAGAGATTTATCCTGGAAGTGGTG
ATACTTACTACAATGAGAAGTTCAAGGGCAAAGCCACACTGACTGCTGACAAATCC
TCCAACACAGCCTACATGCACCTCAGCAGCCTGACATCTGAGGACTCTGCGGTCTTT
TTCTGTGCAAGAAGGAAATTCTATGATGGTAACTACGTCAGGTACTTCGATGTCTGG
GGCGCAGGGACCACTCTCACAGTCTCCTCA (SEQ ID NO: 413)
NP3_C8
GAGGTTCAGCTGCAGCAGTCTGGGGCTGAACTGGCAAGACCTGGGGCCTCAGTAAA
GATGTCCTGCAAGGCTTCTGGCTACACCTTTACTAGCTACACGATGCACTGGGTAAA
ACAGAGGCCTGGACAGGGTCTGGAATGGCTTGGATACTTTAATCCTAGCAGTGGTTA
TACTAATTACGTTCAGAGGTTCAGGGACAAGGCCACATTGACCGCAGACAAATCCTC
CAGCACAGCCTACATGCAACTGAGCAGCCTGACATCTGAGGACTCTGCAGTCTATTA
CTGTGCAAGAGCGGGCGAGGGCTATGCTATGGACTACTGGGGTCAAGGAACCTCAG
TCACCGTCTCCTCA (SEQ ID NO: 414)
NP3_C9
CAGATCCAGTTGGTGCAGTCTGGACCTGAACTGAAGAAGCCTGGAGAGACAGTCAA
GATCTCCTGCAAGGCTTCTGGTTATACCTTCACAGACTATTCAATACACTGGTGAAG
CAGGCTCCAGGAAAGGATTTAAAGTGGGTGGGCTGGATAAACACTGAGACTGGTGA
GCCAACATATGCAGATGACTTCGAGGGACGGTTTGCCTTCTCTTTGGAAACCTCTGC
CAGCACTGCCTTCTTGCAGATCAACAACCTCAAAAATGAGGACACGGCTACATATTT
CTGTAGTAGAGGGGCGGCTACGGGGTTCTGGGGCCAAGGCACCACTCTCACAGTCT
CCTCA (SEQ ID NO: 415)
NP3_C10
CAGGTTACTCTGAAAGAGTCTGGCCCTGGGATATTGAAGCCCTCACAGACCCTCAGT
CTGACTTGTTCTTTCTCTGGGTTTTCACTGAGCACATCTGGTTTGGGTGTAGGCTGGA
TTCGTCAGCCTTCAGGGAAGGGTCTGGAGTGGCTGGCACACATTTGGTGGGATGACG
ATAAGTACTATAACCCATCCCTGAAGAGCCAGCTCACAATCTCCAAGGAAACCTCC
AGAAACCAGGTATTCCTCAAGATCAGCAGTGTGGACAGTGCAGATACTGCCACTTA
CTACTGTGCTCGAACCGTTTACCACGGGGCTATGGACTACTGGGGTCAAGGAACCTC
AGTCACCGTCTCCTCA (SEQ ID NO: 416)
NP3_C11
GAGGTGCAGCTTGTTGAGTCTGGGGGAGGCTTAGTGAGGCCTGGAGGGTCCCTGAA
ACTCTCCTGTGCAGCTTCTGGATTCACTTTCAGTGACTATTACATGTATTGGGTTCGC
CAGACTCCGGAAAAGAGGCTGGAGTGGGTCGCAACCATTAGTGATGGTGGTGGATA
CAATTACTATCCAGACAGTGTGAAGGGGCGATTCACCGTCTCCAGAGACAATGCCA
AGAACAACCTGTACCTGCAAATGAGCAGTCTGAAGTCTGAGGACACAGCCATGTAT
TACTGTGTAAGAGATAAGTACGATGGTTGGGGGTCTGATTTCGATGTCTGGGGCGCA
GGGACCACGGTCACCGTCTCCTCA (SEQ ID NO: 417)
NP3_D1
GATGTGCAGCTGCAGGAGTCTGGACCTGAGCTGGCGAAGCCTGGGGCTTCAGTGAA
GGTATCCTGCAAGGCTTCTGGTTATACATTCACTAGCTACAACATATACTGGGTGAA
GCAGAGCCATGGAAAGAGCCTTGAGTGGATTGGATATATTGATCCTTACAATGGTG
GTACTAGCTACAACCAGAAGTTCAAGGGCAAGGCCACATTGACTGTTGACAAGTCC
TCCAGCACAGCCTACATGCATCTCAACAGCCTGACATCTGAGGACTCTGCAGTCTAT
TACTGTGCCCTCCCATGGGACTACTGGGGCCAAGGCACCTCAGTCACCGTCTC CTCA
(SEQ ID NO: 418)
NP3_D4
GAGGTGCAGCTTGTTGAGTCTGGACCTAGCCTCGTAAAACCTTCTCAGACTCTGTCC
CTCACCTGTTCTGTCACTGGCGACTCCATCACCAGTGGTTACTGGAACTGGATTCGG
AAATTCCCAGGGAATAAATTTGAGTTCATGGGGTACATGAGGTACAGTGGTAGCAC
TTACTTCAGTCCATCTCTCAAAAGTCGAATCTCCATCACTCGAGACACATCCAAGAA
CCACTACTACCTGCAGTTGAGTTCTGTGACTACTGAGGACACAGCCACATATTACTG
TGCAAGGGGGCTAAGGTTTTTCTATGCTATGGACTACTGGGGTCAAGGAACCTCAGT
CACCGTCTCCTCA (SEQ ID NO: 419)
NP3_D5
GATGTGCAGCTGCAGGAGTCTGGGCCTGAGCTGGTGAGGCCTGGGGTCTCAGTGAA
GATTTCCTGCAAGGGTTCCGGCTACACATTCACTGATTATGTTATGCACTGGGTGAA
GCAGAGTCATGCAAAGAGTCTAGAGTGGATTGGAGTTATTAGTACTTACTCTGGTAA
TACAAACTACAACCAGAAGTTTAAGGGGAAGGCCACAATGACTGTAGACAAATTCT
CCAACACAGCCTATATGGAACTTGCCAGATTGACATCTGAAGATTCTGCCATCTATT
ACTGTACAAGGGGGACTGGGGACTTTGACTACTGGGGCCAAGGCACCACTCTCACA
GTCTCCTCA (SEQ ID NO: 420)
NP3_E2
GAGGTTCAGCTGCAGCAGTCTGGGCCTGAGGTGGTGAGGCCTGGGGTCTCAGTGAA
GATTTCCTGCAAGGGTTCCGGCTACACATTCACTGATTATGCTCTGCACTGGGTCAA
GCAGAGTCATACAAAGAGTCTAGAGTGGATTGGAGTTATTAGTACGTACAGTGGTA
CTACAAACTACAACCAGAACTTTAAGGGCAAGGCCACAATGGCTGTAGACAAATCC
TCCAGCACAGCCTATATGGAACTTGCCAGATTGACATCTGAGGATTCTGCCATCTAT
TACTGTGCAAGTGGGGGTAACTACGGTTTGGACTACTGGGGTCAAGGAACCTCAGTC
ACCGTCTCCTCA (SEQ ID NO: 421)
NP3_E3
CAGATCCAGTTGGTGCAGTCTGGACCTGACCTGAAGAAGCCTGGAGAGACAGTCAA
GATCTCCTGCAAGGCTTCTGGTTATACCTTCACAGACTATTCAATGCACTGGGTGAA
GCAGGTTCCAGGAAAGGGTTTAAAGTGGATGGGCTGGATAAACACTGAGACTGATA
AACCAACATATGCAGATGACTTCAAGGGACGGTTTGCCTTCTCTTTGGAAACCTCTG
CCAGCACTGCCTATTCGCAGATCAACAACCTCAAAAATGAGGACACGGCTACATAT
TTCTGTGCTAGACACTATAAATACGACGAGGCCTGGTCACCTTACTGGGGCCAAGGG
ACTCTGGTCACTGTCTCT (SEQ ID NO: 422)
NP3_E4
GAGGTGCAGCTTGTTGAGTCTGGACCTGGCCTGGTGGCGCCCTCACAGAGCCTGTCC
ATCACATGCACTGTCTCAGGGTTCTCATTAACCAGCTATGGTGTAAGCTGGGTTCGC
CAGCCTCCAGGAAAGGGTCTGGAGTGGCTGGGAGTAATATGGGGTGACGGGAGCAC
AAATTATCATTCAGCTCTCATATCCAGACTGAGCATCAACAAGGATAACTCCAAGAG
CCAGGTTTTCTTAAGACTGAACAGTCTGCAAACTGATGACACAGCCACGTACTACTG
TGCCAAAGAGGATGATGGTTACCTGTTCTACTTTGACTACTGGGGCCAAGGCACCAC
TCTCACAGTCTCCTCA (SEQ ID NO: 423)
NP3_E5
GAAGTGCAGCTGGTGGAGTCTGGGGGAGGCTTAGTGAAGCCTGGAGGGTCCCTGAA
ACTCTCCTGTGCAGCCTCTGGATTCACTTTCAGTGACTATTACATGTCTTGGGTTCGC
CAGACTCCGGAAAAGAGGCTGGAGTGGGTCGCAACCATTAGTGATGGTGGTAATTA
CACCTACTATCCAGACAGTGTGAAGGGGCGATTCACCATCTCCAGAGACAATGCCA
AGAACAACCTGTACCTGCAAATGAGCAGTCTGAAGTCTGAGGACACAGCCATGTAT
TACTGTGCAAGAGGGTCTACCTATGGTAACCCTCTTGACCACTGGGGCCAAGGCACC
ACTCTCACAGTCTCCTCA (SEQ ID NO: 424)
NP3 _E6
GAGGTTCAGCTGCAGCAGTCTGGAGCTGAGCTGATGAGGCCTGGGGCCTCAGTGAA
GATATCCTGCAAGGCTACTGGCTACACATTCAGTTCCTACTGGATAGAGTGGGTAAA
GCAGAGGCCTGGACATGGCCTTGAGTGGATTGGAGAGATTTTACCTGGAAGTGATA
ATACTAACTACAATGAGAAGTTCAAGGGCAAGGCCACACTCACTGCAGATACATCC
TCCAACACAGCCTACATGCATCTCAGCAGCCTGACATCTGAGGACTCTGCCGTCTAT
TCCTGTGCAAGGCGATTGTTTCTACGACGTTACTTCGATGTCTGGGGCGCAGGGACC
ACGGTCACCGTCTCCTCA (SEQ ID NO: 425)
NP3_E11
GAGGTTCAGCTGCAGCAGTCTGGGCCTGAGCTGGTGAGGCCTGGGGTCTCAGTGAA
GATTTCCTGCAAGGGTTCCGGCTACACATTCACTGATTATGTTATGCACTGGGTGAA
GCAGAGTCATGCAAAGAGTCTAGAGTGGATTGGAGTTATTAGTACTTACTCGGTAAT
ACAAACTACAACCAGAAGTTTAAGGGCAAGGCCACAATGACTGTAGACAAATCCTC
CAACACAGTCTATATGGAACTTGCCAGATTGACATCTGAGGATTCTGCCATCTATTA
CTGTACAAGGGGGACTGGGGACTTTGACTACTGGGGCCAAGGCACCACTCTCACAG
TCTCCTCA (SEQ ID NO: 426)
NP3_F4
GAGGTGCAGCTTGTTGAGTCTGGACCTGAGCTGAAGAAGCCTGGAGAGACAGTCAA
GATCCCCTGCAAGACTTCTGGGTATACCTTCACAAACTATGGAATGAACTGGGTGAA
GCAGGCTCCAGGAAAGGGTTTAAAGTGGATGGGCTGGATTAACACCAACACTGGAG
AGCCGACATATGCTGAGGATTTCAAGGGACGGTTTGCCTTCTCTTTGGAAACCTCTG
CCAGCACTGCCTATTTGCAGATCAGCAACCTCAAAAATGAGGACACGGCTACATTTT
TCTGTGCAAGAGGATCCATCTATGATGGTTACTACGAGGGGCTTTACTACGGGGCCA
AGGCACCACTCTCACAGTCTCCTCA (SEQ ID NO: 427)
NP3_F7
GAGGTTCAGCTGCAGCAGTCTGGGGCTGAGCTGGTGAGGCCTGGGTCCTCAGTGAA
GATTTCCTGCAAGGCTTCTGGCTATGCATTCAGTAGCTACTGGATGAACTGGGTGAA
GCAGAGGCCTGGACAGGGTCTTGAGTGGATTGGACAGATTTATCCTGGAAATGGTG
ATACTTACTACAATGGTAAGTTCATGGGTAAAGCCACACTGACTGCAGACAAATTTT
CCAGCACAGCCTACATGCAGCTCAGCGGCCTAACTTCTGAGGACTCTGCGGTCTATT
TCTGTGCAAGAAGGCCACTTTTCTATGCTATGGACTACTGGGGTCAAGGAACCTCAG
TCACCGTCTCCTCA (SEQ ID NO: 428)
NP3_F8
CAGGTGCAGCTGCAGCAGTCTGGACATGGCCTGGTGGCGCCCTCACAGAGCCTGTC
CATCACATGCACCGTCTCAGGGTTCTCATTAAGTAGCTATGGTGTACATTGGGTTCG
CCAGCCTCCAGGAAAGGGTCTGGAGTGGCTGGCAGTGATATGGAGTGATGGAAATA
CAATGTATAATTCAGTTCTCAAGTCCAGACTGAACATCAGCAGGGACTACTCCAAGA
GCCAAGTTTTCTTAAAAGGGAACAGTCTCCAAACTGATGACACAGCCATGTACTACT
GTGCCAGAAATTCCGGCTACAATGATTACGACGGATATTATTTCGATGTCTGGGGCG
CAGGGACCTCAGTCACCGTCTCCTCA (SEQ ID NO: 429)
NP3_F10
GAGGTTCAGCTGCAGCAGTCTGGGGCTGAGCTTGTGGAGCCTGGGGCTCCAGTGAA
GCTGTCCTGCAAGGCTTTCGGCTACACCTTCACCAGCTACTGGATGAACTGGGTGAA
GCAGAGGCCTGGACGAGGCCTCGAGTGGATTGGAAGGATTGATCCCTCCGATAGTG
AAACTCACTACAATCAAAACTTCAAGGACAAGGCCGCACTGACTGTTGACAGATCC
TCCAGCACAGCCTACATCCATCTCAGCAGCCTGACATCTGAGGACTCTGCGGTCTAT
TATTGTGCAAGAGAGGTGACTACGGCTACGAAGGGGGTTTTTTCCTGGCTTGCTTAT
TGGGGCCAAGGGACTCTGGTCACTGTCTCT (SEQ ID NO: 430)
NP3_G1
GAGGTGCAGCTTGTTGAGTCTGGGGCTGAGCTGGTGAAGCCTGGGGCTTCAGTGAA
ATTGTCCTGCAAGGCTTCTGGCTACACCTTCACCAGCTACTATATGTACTGGGTGCA
GCTGAGGCCTGGACAAGGCCTTGAGTGGATTGGAGAGATTAATCCCAGCAATGGTG
GTACTAACTTCAATGAGAAGTTCAAGAGCAAGGCCACACTGACTGTAGACAAGTCC
TCCAGCACAGCATACATGCAACTCAGCAGCCTGACATCTGAGGACTCTGCGGTCTAT
TACTGTACAAGATTAGAAGGGTATGGTGACTACGACTATGCTATGTACTACTGGGGT
CAAGGAACCTCAGTCACCGTCTCCTCA (SEQ ID NO: 431)
NP3_G5
GAGGTGCAGCTTGTTGAGTCTGGACCTGAGCTGAAGAAGCCTGGAGAGCCAGTCAG
GATCTCCTGCAAGGCTTCTGGGTATACTTTTACAGACTATGGAATGAACTGGGTGAA
GCAGGCTCCAGGAAAGGGTTTAAAGTGGATGGGCTGGATTAACACCTACACTGGAG
AGCCAACATATGCTGATGACTTCAAGGGCCGGTTTGCCTTCTCTTTGGAAACCTCTG
CCAGCACTGCCTTTTTGCAGATCACCAACCTCAAAAATGAGGACACGGCTACATATT
TCTGTGCAAGAGCCCCATTACTACGGTCCTACTTTGACTACTGGGGCCAAGGCACCT
CAGTCACCGTCTCCTCA (SEQ ID NO: 432)
NP3_G6
GAGGTGCAGCTTGTTGAGTCTGGGGGAGGCTTAGTGAAGCCTGGAGGGTCCCTGAA
ACTCTCCTGTGCAGCCTCCGGATTCACTTTCAGTGACTATTACATGTCTTGGGTTCGC
CAGACTCCGGAAAAGCGGCTGGAGTGGGTCGCAACCATTAGTGATGGTGGTAGTTA
CACCTACTACCCAGACAGTGTGAAGGGGCGATTCACCATCTCCAGAGACAATGCCA
AGAACAACCTGTACCTGCACATGAGCAGTCTGAAGTCTGAGGACACAGCCATGTAT
TACTGTGCAAGAGGGTCTACCTATGGTAACCCTCTAGACTACTGGGGCCAAGGCACC
TCAGTCACCGTCTCCTCA (SEQ ID NO: 433)
NP3_G7
CAGATCCAGTTGGTGCAGTCTGGGGCTGAGCTGGTGAGGCCTGGGTCCTCAGTGAA
GATTTCCTGCAAGGCCTCTGGCTATGCATTCAGTAGCTACTGGATGAACTGGGTGAA
GCAGAGGCCTGGACAGGGTCTTGAGTGGATCGGACAGATTTATCCTGGAAATGGTG
ATACTTACTACAATGGTAAGTTCATGGGTAAAGCCACACTGACTGCAGACAAATTTT
CCAGCACAGCCTACATGCAGCTCAGCAGCCTAACTTCTGAGGACTCTACGGTCTATT
TCTGTGCAAGAAGGCCACTTTTCTATGCTATGGACTACTGGGGTCAAGGAACCTCAG
TCACCGTCTCCTCA (SEQ ID NO: 434)
NP3_G8
CAGGTGCAGCTGCAGCAGTCTGGACCTGAGCTGAAGAAGCCTGGAGAGACAGTCAA
GATCTCCTGCAAGGCTTCTGGTTATACCTTCACCGACTATTCAATGCACTGGGTGAA
GCAGGCTCCAGGAAAGGGTTTAAAGTGGTTGGGCTGGAAAAACACTGAGAGTGGTG
AGGAAACATTTGCAGATGACTTCAAGGGACGGTTTGACTTCTCTTTGGAAACCTCTG
CCAGCACTGTCTATTTGCAGATCAAAAACCTCAAAAATGAAGACACGGCTGCATATT
TCTGTGCTAGATCGGGGGACATCTATGCTATGGACTACTGGGGTCAAGGAACCACG
GTCACCGTCTCCTCA (SEQ ID NO: 435)
NP3_G9
GAGGTTCAGCTGCAGCAGTCTGGGGCTGAGCTGGTGAGGCCTGGGTCCTCAGTGAA
GATTTCCTGCAAGGCTTCTGGCTATGCGTTCAGTAACTACTGGATGAACTGGGTGAG
GCAGAGGCCTGGACAGGGTCTTGAGTGGATTGGACAGATTTATCCTGGAAATGATG
ATACTTACTACAATGGAAAGTTCAAGGGTAAAGTCACACTGACTGCAGACAAGTCC
TCCACCACAGCCTACATCCAGCTCATCAGCCTAACATCTGAGGACTCTGCGGTCTAT
TTCTGTGCAAGAAGGCCACTTTTCTATGCTATGGACTACTGGGGTCAGGGAACCTCA
GTCACCGTCTCCTCA (SEQ ID NO: 436)
NP3_G12
GAGGCTCAGCTGCAGCAGTCTGGAGCTGAGCCGATGAAGCCTGGGGCCTCAGTGAA
GATATCCTGCAAGACTACTGGCTACACATTCACTAGCGACTGGATAGAGTGGGTACA
ACAGAGGCCTGGACATGGCCTTGAGTGGATTGGAGAGATTTTACCTGGGAGTGGTA
ATACTAACTACAATGAGAACTTCAAGGGCAAGGCCACACTCACTGCAGATACATCC
TCCAACACAGCCTACATGCATCTCAGCAGCCTGACATCTGAGGACTCTGCCGTCTAT
TACTGTGCAAGGCGATTGTTTCTACGACGGTACTTCGATGTCTGGGGCGCAGG
GACCACGGTCACCGTCTCCTCA (SEQ ID NO: 437)
NP3_H2
GAGGTTCAGCTGCAGCAGTCTGGGGCTGAACTGGCAAGACCTGGGGCCTCAGTGAA
GATGTCCTGCAAGGCTTCTGGCTACATCTTTACTAGCTACACGATCCACTGGATAAA
ACAGAGGCCTGGACAGGGTCTGGAATGGATTGGATACTTTAATCCTAGCAGTGATTA
TTTTAGTTACATTCAGATCTTCAAGGACAAGGCCACATTGACTGCAGACAAATTCTC
CAGCACAGCCTACATGCAACTGAGCAGCCTGACATCTGAGGACTCTGCAGTCTATTA
CTGTGCAAGAGCGGGCGAGGTCTATGCTAAGGACTACTGGGGTCAAGGAACCTCAG
TCACCGTCTCCTCA (SEQ ID NO: 438)
NP3_H9
CAGATCCAGTTGGTGCAGTCTGGACCTGAGCTGAAGAAGCCTGGAGAGACAGTCAA
GATCTCCTGCAAGGCTTCTGGTTATACCTTCACAGACTATCCAATACACTGGGTGAA
GCAGGCTCCAGGAAAGGGTTTAAAGTGGATGGGCTGGATAAACGCTGAGACTGGTG
AGCCAACATATGCAGATGACTTCAAGGGACGGTTTGTCGTCTCTTTGGAAACCTCTG
CCAGTACTGCCTATTTACAGATCAACAACCTCAAAAATGAGGACACGGCTACTTTTT
TCTGTGCTAGACACTATAGGTACGACGAGGCCTGGTTTGTTTATTGGGGCCAAGGGA
CTCTGGTCACTGTCTCTGCAGCTAGCACCAAGGGCCCATCGGTCACCGTCTCCTCA
(SEQ ID NO: 439)
NP3_H12
CAGATCCAGTTGGTACAGTCTGGACCTGAGCTGAAGAAGCCTGGAGAGACAGTCAA
GATCTCCTGCAAGGCTTCTGGGTATATGTTCACAAACTATGGAATGAACTGGGTGAA
ACAGGCTCCAGGAAAGGGTTTAAAGTGGATGGGCTGGATAAACACCTACACTGGAG
AGCCAACATATGCTGATGACTTCAAGGGACGGTTTGCCTTCTCTTTGGATATTTCTGG
CAGTACTGCCTATTTGCAGATCAACAACCTCAAAAATGAGGACATGGCTACATATTT
CTGTGCGAGAACGAGGGAAATATGGGGTAACGTCGATTTTCCTTACTGGGGCCAAG
GGACTCTGGTCACTGTCTCT (SEQ ID NO: 440)
Nucleotide sequences corresponding to the Light Chain sequences
described in Example 1 are described below.
Light Chain Nucleotide Sequences
NP3_A3
GACATTGTGATGACTCAGTCTCCAGCCTCCCTATCTGTATCTGTGGGAGAAACTGTC
ACCATCACATGTCGATCAAGTGAGAATATTTACAGTAATTTAGCGTGGTTTCAACAG
AAACAGGGAAAATCTCCTCAGCTCCTGGTCTATGGTGCAACAGACTTAGCAGATGGT
GTGCCATCAAGGTTCAGTGGCAGTGGATCGGGCACACAGTATTCCCTCAAGATCAAC
AGCCTGCAGTCTGAAGATTTTGGGACTTATTACTGTCAACATTTTTGGGGCACTCCGT
GGACGTTCGGTGGAGGGACCAAGCTGGAAATCAAA (SEQ ID NO: 441)
NP3_A4
GACATTGTGATGACCCAGTCTCACAAATTCATGTCCACATCAGTAGGAGACAGGGTC
AGCATCACCTGCAAGGCCAGTCAGGATGTGAGTACTGCTGTAGCCTGGTATCAACA
GAAACCAGGACAATCTCCTAAACTACTGATTTATTCGGCATCCCACCGGTACACTGG
AGTCCCTGATCGCTTCACTGGCAGTGGATCTGGGACGGATTTCACTTTCACCATCAG
CAGTGTGCAGGCTGAAGACCTGGCAGTTTATTACTGTCAGCAACATTATAGTAATCC
GCTCACGTTCGGTGCTGGGACCAAGCTGGAAATCAAA (SEQ ID NO: 442)
NP3_A6
GACATCCAGATGAACCAGTCTCCATCCTCCCTATCTGTATCTGTGGGAGAAACTGTC
ACCATCACATGTCGAACAAGTGAGAATATTTACAGTAATTTAGCATGGTATCAGCAG
AAACAGGGAAAATCTCCTCAGCTCCTGGTCTATGGTGCAACAGACTTAGCAGATGGT
GTGCCATCAAGGTTCAGTGGCAGTGGATCAGGCACACAGTATTCCCTCAAGATCAGC
AGCCTGCAGTCTGAAGATTTTGGGACTTATTACTGTCAACATTTTTGGGGTACTCCGT
GGACGTTCGGTGGAGGCACCAAGCTGGAAATCAAA (SEQ ID NO: 443)
NP3_A7
GACATCCAGATGACTCAGTCTCCAGCCTCTGTACCTGTCACTCCTGGAGAGTCAGTA
TCCATCTCCTGCAGGTCTAGTAAGAGTCTCCTGCATAGTAATGGCAACACTTACGTG
TATTGGTTCCTGCAGAGGCCAGGCCAGTCTCCTCGGCTCCTGATATATCGGATGTCC
AACCTTGCCTCAGGAGTCCCAGACAGGTTCAGTGGCAGTGGGTCAGGAACTGCTATC
ACACTGAGAATCAGTAGAGTGGAGGCTGAGGATGTGGGTGTTTATTCCTGTATGCAA
CATCTAGAATATCCTCTCACGTTCGGTGCTGGGACCAAGCTGGAAATAAAA (SEQ ID
NO: 444)
NP3_A8
GACATTGTGATGACCCAGTCTCCTGCTTCCTTAGTTGTATCTCTGGGGCAGAGGGCC
ACCATCTCATGCAGGGCCAGCAAAAGTGTCAGTGCTTCTGGCTATAGTTATATACAC
TGGTACCAACAGATTCCAGGACAGCCACCCAAACTCCTCATCTATCTTGCATCCAAC
CTAGAATCTGGGGTCCCTGCCAGGTTCAGTGGCAGTGGGTCTGGGACAGACTTCACC
CTCAACATCCATCCTGTGGAGGAGGAGGATGCTGCAACCTATTACTGTCAGCACAGT
AGGGAGCTTCCGCTCACGTTCGGTACTGGCACCAAGCTGGAAATCAAA (SEQ ID NO:
445)
NP3_A9
GAAAATGTTCTCACCCAGTCTCCAGCCTCCAATCCAGTCACTCTTGGAACATCAGCT
TCCATCTCCTGCAGGTCTAGTAAGAGTCTCCTACATAGTAATGGCTTCACTTATTTGT
ATTGGTATCTGCAGAAGCCAGGCCAGTCTCCTCAGCTCCTGATTTATCAGATGTCCT
ACCTTGCCTCAGGAGTCCCAGACAGGTTCAGTAGCAGTGGGTCAGGAACTGATTTCA
CACTGAGGATCAGCAGAGTGGAGGCTGAGGATGTGGGTGTTTATTACTGTGCTCAA
AATCTAGAACTTCCGTTCACGTTCGGCTCGGGCACCAAGCTGGAAATCAAA (SEQ ID
NO: 446)
NP3_A10
GAAAATGTTCTCACCCAGTCTCCAGCTTCTTTGACTGTCTCTCTAGGACAGAGGGCC
ACCCTCTCCTGCAAGGCCAGCCAAAGTGTTGATTATGATGGTGATAGTTATATGAAC
TGGTACCAACAGAAACCAGGTCAGCCACCCAAACTCCTCATCTATGCTGCATCCTAT
CTACAATCTGGGATCCCAGCCAGGTTTAGTGGCAGTGGGTCTGGGACAGACTTCACC
CTCAACATCCATCCTGTGGAGGAGGAGGATGCTGCAACCTATTACTGTCAGCAAAGT
ATTGAGGATCCGTACACGTTCGGAGGGGGCACCAAGCTGGAAATCAAA (SEQ ID NO:
447)
NP3_A11
GACATCCAGATGACCCAGTCTCCATCCTCCCTATCTGTTTCTGTGGGAGAGACTGTC
ACCATCACTTGTCGAGCAAGTGAGAATATTTACAGTAGTTTGGCATGGTATCAGCAG
AAACAGGGAAAATCTCCTCAGCTCCTGGTCTATACTGCAACAGACTTAGCAGATGGT
GTGCCGTCAAGGTTCAGTGGCAGTGGATCAGGCTCGCAGTATTCCCTCAGGATCAAC
AGCCTGCAGTCTGAAGATTTTGGGAGTTATTACTGTCAACATTTTTGGGGTTTTCCGT
GGACGTTCGGTGGAGGCACCAAGCTGGAAATCAAA (SEQ ID NO: 448)
NP3_B1
GATGTTGTGATGACCCAGACTCCACTCACTTTGTCGGTTACCATTGGACAACCAGCC
TCCATCTCTTGCAAGTCAAGTCAGACCCTCTTAGATAGTGATGGAAAGACATATTTG
AATTGGTTGTTCCAGAGGCCAGGGCAGTCTCCAAAACGCCTAATCTATCTGGTGTCT
GAACTGGACTCTGGAGTCCCTGACAGGTTCACTGGCAGTGGATCAGGGACAGATTTC
ACACTGAAAATCAGCAGAGTGGAGGCTGAGGATTTGGGAGTTTATTATTGCTGGCA
AGGTACACATTTTCCTCAGACGTTCGGTGGAGGGACCAAGCTGGAGCTGAAA (SEQ
ID NO: 449)
NP3_B2
GATATCCAGATGACACAGACTACATCCTCCCTGTCTGCCTCTCTGGGAGACAGAGTC
ACCATCAATTGCAGTGCAAGTCAGGGCATTAGCAATTATTTAAACTGGTATCAGCAG
AAACCAGATGGAACTGTTAAACTCCTGATCTATTTCACATCAAGTTTACACTCTGGG
TTCCCATCGAGGTTCAGTGGCAGTGGGTCTGGGACAGATTTTTCTCTCACCATCAGC
AACCTGGAACCTGAAGATATTGCCACTTACTATTGTCAGCAATATAGTAAGCTTCCA
TTCACGTTCGGCTCGGGGACAAAGTTGGAAATAAAA (SEQ ID NO: 450)
NP3_B3
ATTGTGCTGACCCAATCTCCAGCTTCTTTGGCTGTGTCTCTAGGGCAGAGGGCCACC
ATCTCCTGCAAGGCCAGCCAAAGTGTTGATTATGATGGTGATAGTTATATGAACTGG
TACCAACAGAAACCAGGACAGCCACCCAAACTCCTCATCTATGCTGCATCCAATCTA
GAATCTGGGATCCCAGCCAGGTTTAGTGGCAGTGGGTCTGGGACAGACTTCACCCTC
AACATCCATCCTGTGGAGGAGGAGGATGCTGCAACCTATTACTGTCAGCAAAGTAA
TGAGGATCCGTACACGTTCGGAGGGGGGACCAAGCTGGAGCTCAAA (SEQ ID NO:
451)
NP3_B4
GACATCCAGATGACCCAGTCTCCATCCATCATGTCTGCATCTCCAGGGGAAAAGGTC
ACCATGACCTGCAGTGCCAGCTCAAGTGTAAGTTACATGCACTGGTACCAGCAGAA
GTCAAGCACCTCCCCCAAACTCTGGATTTATGACACATCCGAACTGGCTTCTGGAGT
CCCAGGTCGCTTCAGTGGCAGTAGGTCTGGAAACTCTTACTCTCTCACGATCAGCAG
CATGGAGGCTGAAGATGTTGCCACTTATTACTGTTTTCAGGGGAGTGGGTATCCACT
AACGTTCGGAGGGGGGACCAAGCTGGAGCTGAAA (SEQ ID NO: 452)
NP3_B5
GAAAATGTTCTCACCCAGTCTCCAGCAATCATGTCTGCATCTCCAGGGGAGAAGGTC
ACCATGACCTGCAGTGCCAGCTCAAGTGTAAATTACATGCACTGGTACCAGCAGAG
GCCAGGATCCTCGCCCAAACCCTGGATTTATGACACATCCAACCTGGCTTCTGGAAT
CCCTGTACGCTTCAGTGGCAGTGGATCTGGGACCTCTTACTCTCTCATAATCAGCAG
CATGGAGGCTGAAGATGCTGCCACTTATTACTGCCATCAGCGGAGTAGTTTCCCATT
CACATTCGGCTCGGGGACAAAGTTGGAAATCAAA (SEQ ID NO: 453)
NP3_B8
GACATCCAGATGAACCAGTCTCCATCCTCCCTGTCTGCCTCTCTGGGAGGCAGAGTC
ACCATCAGTTGCAGTGCAAGTCAGGGCATTAGCAATTATTTAAACTGGTATCAGCAG
AAACCAGATGGAACTGTTAAACTCCTGATCTATTACACATCAAGTTTACACTCAGGA
GTCCCATCAAGGTTCAGTGGCAGTGGGTCTGGGACAGATTATTCTCTCACCATCAGC
AACCTGGAACCTGAAGATATTGCCACTTACTATTGTCAACAGTATAATAAGCTTCCG
TACACGTTCGGAGGGGGCACCAAGCTGGAAATCAAA (SEQ ID NO: 454)
NP3_B9
GACATCCAGATGACTCAGTCTCCAGCCTCCCTATCTGTATCTGTGGGAGAAACTGTC
ACCATCACATGTCGAGCAAGTGAGAATATTTACAGTAATTTAGCATGGTATCAGCAG
AAACAGGGAAAATCTCCTCAGCTCCTGGTCAATGGTGCAACAGACTTAGCAGATGG
TGTGCCCTCAAGGTTCAGTGGCAGTGGATCAGGCACACAATATTCCCTCAAGATCAA
CAGCCTGCAGTCTGAAGATTTTGGGAGTTATTACTGTCAACTTTTTTGGGGTACTCCG
TGGACGTTCGGTGGAGGGACCAAGCTGGAAATCAAA (SEQ ID NO: 455)
NP3_B10
GACATTGTGATGACACAGTCTCCATCCTCTTTGGCTGTATCTCTAGGGCAGAGGGCC
ACCATCTCCTGCAAGGCCAGCCAAAGTGTTGATTTTGATGGTAAGAGTTATATGAAC
TGGTACCAACAGAAACCAGGACAGCCACCGAAACTCCTCATCTATGTTGCATCCAAT
CTAGAATCTGGGATTCCTGCCAGGTTTAGTGGCAGTGGGTCTGGGACAGATTTCACC
CTCAACATCCATCCTGTGGAAGAAGAGGATGCTGCAACCTATTACTGTCAGCAAACT
AATGAGGATCCGCTCACGTTCGGTACTGGCACCAAGCTGGAAATCAAA (SEQ ID NO:
456)
NP3_B12
GACATTGTGCTGACCCAATCTCCAGCTTCTTTGGCTGTGTCTCTAGGGCAGAGGGCC
ACCATCTCCTGCAAGGCCAGCCAAAGTGTTGATTATGATGGTTATAGTTATATGAAC
TGGTACCAACAGAAACCAGGACAACCACCCAAACTCCTCATCTATGCTGCATCCAAT
CTAGAATCTGGGATCCCAGCCAGGTTTAGTGGCAGTGGGTCTGGGACAGACTTCACC
CTCAACATCCATCCTGTGGAGGAGGAGGATGCTGCAACCTATTACTGTCAGCAAAGT
AATGCGGATCCGTTCACGTTCGGAGGGGGGACCAAGCTGGAGCTGAAA (SEQ ID NO:
457)
NP3_C3
GACATTGTGATGACTCAGTCTCCAGCCTCCCTATCTGTATCTGTGGGAGAAACTGTC
ACCATCACATGTCGAGCAAGTGAAAATATTTACAGTAATTTAGCATGGTATCAGCAG
AAACAGGGAAAATCTCCTCAGCTCCTGGTCTATGTTGCGACAGACTTAGCAGATGGT
GTGCCATCAAGGTTCAGTGGCAGTGGATCAGGCACACAGTATTCCCTCAAGATCAAC
AGCCTGCAGTCTGAAGATTTTGGGAGTTATTACTGTCAACATTTTTGGGGTACTCCGT
GGACGTTCGGTGGAGGGACCAAGCTGGAGCTGAAA (SEQ ID NO: 458)
NP3_C4
GATATTGTGATGACTCAGGCTGCACCCACTTTGTCGGTTACCATTGGACAACCAGCC
TCCATCTCTTGCAAGTCAAGTCAGAGCCTCTTAGATAGTGATGGAAAGACATATTTG
AATTGGTTGTTACAGAGGCCAGGCCAGTCTCCAAGGCGCCTAATCTATCTGGTGTCT
AAACTGGACTCTGGAGTCCCTGACAGGTTCACTGGCAGTGGATCAGGGACAGACTT
CACACTAAAAATCAGCAGAGTGGAGGCTGAGGATTTGGGAGTTTATTATTGCTGGC
AAGGTACACATTTTCCTCGGACGTTCGGTGGAGGGACCAAGCTGGAAATCAAA (SEQ
ID NO: 459)
NP3_C5
GACATCCAGATGACTCAGTCTCCAGCCTCCCTATCTGTATCTGTGGGAGAAACTGTC
ACCATCACATGTCGAGCAAGTGAGAATATTTACAGTAATTTAGCATGGTATCAGCAG
AAACAGGGAAAATCTCCTCAGCTCCTGGTCTATGGTGCAACAGACTTAGCAGATGGT
GTGCCATCAAGGTTCAGTGGCAGTGGATCAGGCACACAATATTCCCTCAAGATCAAC
AGCCTGCAGTCTGAAGATTTTGGGAGTTATTACTGTCAACATTTTTGGGGTACTCCGT
GGACGTTCGGTGGAGGGACCAAGCTGGAGCTGAAA (SEQ ID NO: 460)
NP3_C6
GACATCAAGATGACCCAGTCTCCATCCTCTTTGGCTGTGTCTCTAGGGCAGAGGGCC
ACCATCTCCTGCAAGGCCAGCCAAAGTGTTGATTATGATGGTGATAGTTATATGAAC
TGGTACCAACAGAGACCAGGACAGCCACCCAAACTCCTCATCTATGCTGCATCCAAT
CTAGAATCGGGGATCCCAGCCAGGTTTAGTGGCAGTGGGTCTGGGGCAGACTTCAC
CCTCAACATCCATCCTGTGGAGGAGGAGGATGCTGCAACCTATTACTGTCAGCAAAG
TAATGAGGATCCGTGGACGTTCGGTGGAGGGACCAAGCTGGAAATCAAA (SEQ ID
NO: 461)
NP3_C8
GACATTGTGATGACCCAGTCTCCAGCCTCTTTGGCTGTGTCTCTAGGGCAGAGGGCC
ACCATCTCCTGCAAGGCCAGCCAAAGTGTTGATTATGATGGTGATAGTTATATGAGC
TGGTACCTACAGAAACCAGGACAGCCACCCAAACTCCTCATCTATGCTGCATCCAAT
CTAGAATCTGGGATCCCAGCCAGGTTTAGTGGCAGTGGGTCTGGGACAGACTTCACC
CTCAACATCCATCCTGTGGAGGAGGAGGATGCTGCAACCTATTACTGTCAGCAAAGT
ATTGGGGATCCGTGGACGTTCGGTGGAGGCACCAAGCTGGAAATCAAA (SEQ ID NO:
462)
NP3_C9
GACATTGTGCTGACCCAATCTCCAGCTTCTTTGGCTGTGTCTCTAGGGCAGAGGGCC
ACCATCTCCTGCAAGGCCAGCCAAAGTGTTGATTATGATGGTGATAGTTATATGAAC
TGGTACCAACAGAAACCAGGACAGCCACCCAAACTCCTCATCTATGTTGCATCCAAT
CTAGAATCTGGGATCCCTGCCAGGTTTAGTGGCAGTGGGTCTGGGACAGACTTCACC
CTCAACATCCATCCTGTGGAGGATGAGGATGCTGCAACCTATTACTGTCAGCAAACT
AATGAGGATCCGCTCACGTTCGGTGCTGGGACCAAGCTGGAGCTGAAA (SEQ ID NO:
463)
NP3_C10
GACATCAAGATGACCCAGTCTCCATCAATCATGTCTGCATCTCCAGGGGAGAAGGTC
ACCATGACCTGCAGTGCCAGCTCAAGTGTAAGTTACATGTACTGGTTCCAACAGAAG
CCAGGATCCTCCCCCAGACTCCTGATTTATGACACATCCAACCTGGCTTCTGGAGTC
CCTGTTCGCTTCAGTGGCAGTGGGTCTGGGACCTCTTACTCTCTCACAATCAGCCGA
ATGGAGGCTGAAGATGCTGCCACTTATTACTGCCAGCAGTGGAGTGGTTACCCGTAC
ACGTTCGGAGGGGGGACCAAGCTGGAAATCAAA (SEQ ID NO: 464)
NP3_C11
GACATTGTGATGACACAGTCTCCATCCCTCATGTCTACATCTCTAGGGGAACGGGTC
ACCATGACCTGCACTGCCAACTCAAGTGTAAGTTCCAATTACATGCACTGGTACCAG
CAGAAGCCAGGATCCTCCCCCAAACTCTGGATTTATAGCACATCCAACCTGGCTTCT
GGAGTCCCAGCTCGCTTCAGTGGCAGTGGGTCTGGGACCTCTTACTCTCTCACAATC
AGCAACATGGAGGCTGAAGATGCTGCCACTTATTACTGCCACCAGTTTCATCGTTCC
CCACGGACGTTCGGTGGAGGGACAAAGTTGGAAATCAAA (SEQ ID NO: 465)
NP3_D1
GATATTGTGATGACTCAGGCTGCACCCACTTTGTCGGTTACCCTTGGACAACCAGCC
TCCATCTCTTGCAAGTCAAGTCAGAGCCTCTTAGATAGTGATGGAAAGACATATTTG
AGTTGGTTGTTACAGAGGCCAGGCCAGTCTCCAAAGCGCCTAATCTATCTGGTGTCT
GAACTGGACTCTGGAGTCCCTGACAGGTTCACTGGCAGTGGATCAGGGACAGATTTC
ACACTGAAAATCAGCAGAGTGGAGGCTGAGGATTTGGGAGTTTATTATTGCTGGCA
AGGTACACATTTTCCTCAGACGTTCGGTGGAGGGACCAAGCTGGAAATCAAA (SEQ
ID NO: 466)
NP3_D4
GAAAATGTTCTCACCCAGTCTCCAGCTTCTTTGACTGTCTCTCTAGGGCAGAGGGCC
ACCCTCTCCTGCAAGGCCAGCCAAAGTGTTGATTATGATGGTGATAGTTATATGAAC
TGGTACCAACAGAAACCAGGACAGCCACCCAAACTCCTCATCTATGCTGCATCCTAT
CCAGAATCTGGGATCCCAGCCAGGTTTAGTGGCAGTGGGTCTGGGACAGACTTCACC
CTCAACATCCATCCTGTGGAGGAGGAGGATGCTGCAACCTATTACTGTCAGCAAAGT
ATTGAGGATCCGTACACGTTCGGAGGGGGCACCAAGCTGGAAATCAAA (SEQ ID NO:
467)
NP3_D5
GACATTGTGATGACCCAGTCTCACAAATTCATGTCCACATCAGTAGGAGACAGGGTC
AGCATCACCTGCAAGGCCAGTCAGGATGTGGGTACTGCTGTAGCCTGGTATCAACA
GAAACCAGGACAATCTCCTAAACTACTGATTTACTGGGCATCCACCCGGCACACTGG
AGTCCCTGATCGCTTCACAGGCAGTGGATCTGGGACAGATTTCACTCTCACCATTAG
CAATGTGCAGTCTGAAGACTTGGCAGATTATTTCTGTCACCAATATAGCAGCTATCC
GTACACGTTCGGAGGGGGGACCAAGCTGGAAATCAAA (SEQ ID NO: 468)
NP3_E2
GACATTGTGATGACCCAGTCTCACAAATTCATGTCCACATCAGTAGGAGACAGGGTC
AGCATCACCTGCAAGGCCAATCAGGATGTGGGTTCTGCTGTAGCCTGGTATCAGGAG
AAACCAGGGCAATCTCCTAAACTTCTGATTTACTGGACATCCACCCGACACACTGGA
GTCCCTGATCGCTTCACAGGCAATGGATCTGGGACAGATTTCACTCTCACCATTAGC
AATGTCCAGTCTGAAGACCTGGTAGATTATTTCTGTCAGCAATATAGCAGGTATCCT
CTCACGTTCGGCTCGGGGACCAAGCTGGAGCTGAAA (SEQ ID NO: 469)
NP3_E3
GACATTGTGCTGACCCAATCTCCAGCCTCCCTATCTGTATCTGTGGGAGAAACTGTC
ACCATCACATGTCGAGCAAGTGAGAATATTTACAGTAATTTAGCATGGTTCAGCAGA
AACAGGGACAATCTCCTCAGCTCCTAGTCTATGCTGCAACAGACTTAGCAGATGGTG
TGCCATCAAGGTTCAGTGGCAGTGGATCAGGCACACAGTATTCCCTCAAGATCAACA
GCCTGCAGTCTGAAGATTTTGGGAATTATTACTGTCAACATTTTTGGGGTACTCCGTG
GACGTTCGGTGGAGGCACCAAGCTGGAAATCAAA (SEQ ID NO: 470)
NP3_E4
GACATCAAGATGACCCAGTCTCCATCCTCCCTGTCTGCCTCTCTGGGAGACAGAGTC
ACCATCAGTTGCAGTGCAAGTCAGGACATTAGCGATTATTTAAACTGGTATCAGCAG
AAACCAGATGGAACTGTTAAACTCCTGATCTATTACACATCAAGTTTACACTCAGGG
GTCCCATCAAGGTTCAGTGGCAGTGGGTCTGGGACAGATTATTCTCTCACCATCAGC
AACCTGGAACCTGAAGATATTGCCACTTACTATTGTCAGCAGTATAGTAAGCTTCCA
TACACGTTCGGAGGGGGCACCAAGCTGGAAATCAAA (SEQ ID NO: 471)
NP3_E5
GATATCCAGATGACACAGACTACATCCTCCCTGTCTGCCTCTCTGGGAGACAGAGTC
ACCATCAATTGCAGTGCAAGTCAGGGCATTAGCAATTATTTAAACTGGTATCAGCAG
AAACCAGATGGAACTGTTAAACTCCTGATCTATTACACATCAATTTTACACTCAGGA
GTCCCATCAAGGTTCAGTGGCAGTGGGTCTGGGACAGATTATTCTCTCACCATCAGC
AACCTGGAACCTGAAGATATTGCCACTTACTATTGTCAGCAGTATAGTAAGCTTCCA
TTCACGTTCGGCTCGGGGACAAAGTTGGAAATCAAA (SEQ ID NO: 472)
NP3_E6
ATTGTGCTGACCCAATCTCCAGCTTCTTTGGCTTTGTCTCTAGGGCAGAGGGCCACC
ATCTCCTGCAAGGCCAGCCAAAGTGTTGATTATGATGGTGATAGTTATATGAACTGG
TACCAACAGAAACCAGGACAGCCACCCAAACTCCTCATCTCTGCTGCATCCAATCTA
GAATCTGGGATCCCAGCCAGGTTTGGTGGCAGTGGGTCTGGGACAGACTTCACCCTC
AACATCCATCCTGTGGAGGAGGAGGATGCTGCAACCTATTACTGTCAGCAAAGTAA
TGAGGATCCGTTCACGTTCGGAGGGGGCACCAAGCTGGAAATCAAA (SEQ ID NO:
473)
NP3_E11
GACATTGTGATGACCCAGTCTCACAAATTCATGTCCACATCAGTAGGAGACAGGGTC
AGCATCACCTGCAAGGCCAGTCAGGATGTGGGTTCTGCTGTAGCCTGGTATCAACAG
AAACCAGGGCAATCTCCTAAACTACTGATTTACTGGACATCCACCCGGCACACTGGA
GTCCCTGATCACTTCACAGGCAGTGGATCTGGGACAGATTTCACTCTCACCATTAAC
AATGTGCAGTCTGAAGACTTGGCAGATTATTTCTGTCACCAATATAGCAGCTATCCG
TACACGTTCGGAGGGGGGACCAAGCTGGAAATCAAA (SEQ ID NO: 474)
NP3_F4
GACATCAAGATGACCCAGTCTCCATCCTCCCTATCTGTATCTGTGGGAGAAACTGTC
ACCATCACATGTCGAGCAAGTGAAAATATCTACAGTAATTTAGCGTGGTATCAGCAG
AAACAGGGAAAATCTCCTCAGCTCCTGGTCTCTGCTGCAACAAACTTAGCAGATAAT
GTGCCATCAAGGTTCAGTGGCAGTGGATCAGGCACACAGTATTCCCTCAAGATCAAT
AGCCTGCAGTCTGAAGACTTTGGGAGTTATTACTGTCAACATTTTTGGGGTTTTCCTC
GGACGTTCGGTGGAGGCACCAAGCTGGAAATCAAA (SEQ ID NO: 475)
NP3_F7
GACATTGTGCTGACCCAATCTCCAGGTTCTTTGGCTGTGTCTCTAGGGCAGAGGGCC
ACCATCTCCTGCAAGGCCAGCCAAAGTGTTGATTATGATGGTGATAGTTATATGAAC
TGGTACCAACAGAAACCAGGACAGCCACCCAAACTCCTCATCTATGCTGCATCCAAT
CTAGAATCTGGGATCCCAGTCAGGTTTAGTGGCAGTGGGTCTGGGACAGACTTCACC
CTCAATATCCATCCTGTGGAGGAGGAGGATGCTGCAACCTATTACTGTCGCAAAGTA
ATGAGGATCCGTGGACGTTCGGTGGCGGGACCAAGCTGGAGCTGAAA (SEQ ID NO:
476)
NP3_F8
GACATCCAGATGACCCAGTCTCCATCCATAATGGCTGCCTCTGTGGGGCAGAAGGTC
ACCATGACCTGCAGTGCCAGCTCAAGTGTTAGTTCCAGTTACTTGCACTGGTACCAG
CAGAAGTCAGGCGCTTCCCCCAAACCCTTGATTCATAGGACATCCAACCTGGCTTCT
GGAGTCCCACCTCGCTTCAGTGGCAGTGGGTCTGGGACCTCTTACTCTCTCACAATC
AGCAGCGTGGAGGCTGAAGATGATGCAACTTATTACTGCCAGCAGTGGAGTGGTTA
TCCATTCACGTTCGGTACTGGGACAAAGTTGGAAATCAAA (SEQ ID NO: 477)
NP3_F10
GATATCCAGATGACACAGACTACATCCTCCCTGTCTGCCTCTCTGGGAGACAGAGTC
ACCATCAGTTGCAGTGCAAGTCAGGACATTAGCGATTATTTAAACTGGTATCAACAG
AAACCAGATGGAACTATTAAGCTCCTGATCTATTACACATCAAGTTTACACTCAGGA
GCCCCATCAAGGTTCAGTGGCAGTGGGTCTGGGACAGATTATTCTCTCACCATCAGC
AACCTGGAACCTGAAGATATTGCCACTTACTATTGTCAACAGTATAGTAAGCTTCCG
TGGACGTTCGGTGGAGGGACCAAGCTGGAGCTGAAA (SEQ ID NO: 478)
NP3_G1
ATTGTGCTGACACAGTCTCCTGCTTCCTTAGCTGTTTCTCTGGGGCAGAGGGCCACC
ATCTCATGCAGGGCCAGCAAAAGTGTCAGTGCATCTGGCTATAGTTATCTCCACTGG
TACCAACAGAAACCAGGACAGCCACCCAAACTCCTCATCTATCTTGCATCCAACCTA
CCATCTGGGGTCCCTGCCAGGTTCAGTGGCAGTGGGTCTGGGACAGACTTCACCCTC
AACATCCATCCTGTGGAGGAGGAGGATGCTGCAACCTATTACTGTCAGCACCGTCGG
GAGATTCCGCTCACGTTCGGTGCTGGCACCAAGCTGGAAATCAAA (SEQ ID NO: 479)
NP3_G5
GACATTGTGATGACCCAGTCTCACAAATTCATGTCCACATCAGTAGGAGACAGGATC
AGCATCACCTGCAAGGCCAGTCAGGATGTGAGTACTGCTGTAGTCTGGTATCAACAG
AAACCAGGACAGTCTCTTAAACTACTGATTTACTCGGCATCCTACCGGTACACTGGA
GTCCCTGATCGCTTCACTGGCAGTGGATCTGGGACGGATTTCACTTTCACCATCAGC
AGTGTGCAGGCTGAAGACCTGGCAATTTATTACTGTCAGCAATATTATAGTACTCCA
TTCACGTTCGGCTCGGGGACAAAGTTGGAAATCAAA (SEQ ID NO: 480)
NP3_G6
GACATCCAGATGACTCAGTCTCCAGCCTCCCTGTCTGCCTCTCTGGGAGACAGAGTC
ACCATCAATTGCAGTGCAAGTCAGGGCATTAGCAATTATTTAAACTGGTATCAGCAG
AAACCAGATGGAACTGTTAAACTCCTGATCTATTTCACATCAAGTTTACACTCTGGG
TTCCCATCAAGGTTCAGTGGCAGTGGGTCTGGGACAGATTTTTCTCTCACCATCAGC
AACCTGGAACCTGAAGATATTGCCACTTACTATTGTCAGCAATATAGTAAGCTTCCA
TTCACGTTCGGCTCGGGCACCAAGCTGGAAATCAAA (SEQ ID NO: 481)
NP3_G7
GACATCCAGATGACTCAGTCTCCAGCCTCCCTATCTGTATCTGTGGGAGAAACTGTC
ACCATCACATGTCGAGCAAGTGAAAATATTTACAGTAATTTAGCATGGTATCAGCAG
AAACAGGGAAAATCTCCTCAGCTCCTGGTCTATGTTGCGACAGACTTAGCAGATGGT
GTGCCATCAAGGTTCAGTGGCAGTGGATCAGACACACAGTATTCCCTCAAGATCAAC
AGCCTGCAGTCTGAAGATTTTGGGAGTTATTACTGTCAACATTTTTGGGGTACTCCGT
GGACGTTCGGTGGAGGCACCAAGCTGGAAATCAAA (SEQ ID NO: 482) [001420]
NP3_G8
GAAAATGTTCTCACCCAGTCTCCAGCTTCTTTGGCTGTGTCTCTAGGGCAGAGGGCC
ACCATCTCCTGCAAGGCCAGCCAAAGTGTTGATTATGATGGTGGTAGTTATATGAAC
TGGTACCAACAGAAACCAGGACAGCCACCCAAACTCCTCATCTATGCTGCATCCACT
CTAGAATCTGGAATCCCAGCCGGGTTTAGTGGCAGTGGGTCTGGGACAGACTTCACC
CTCAACATCCATCCTGTGGAGGAGGAGGATGCTGCAACCTATTACTGTCAGCAAAGT
AATGAAGATCCGTGGACGTTCGGTGGAGGCACCAAGCTGGAAATCAAA (SEQ ID NO:
483)
NP3_G9
GACATCCAGATGACCCAGTCTCCATCCTCTTTGGCTGTGTCTCTAGGGCAGAGGGCC
AGCATCTCCTGCAAGGCCAGCCAAAGTGTTGATTATGATGGTGAGAGTTATATGAAC
TGGTACCAACAGAAACCAGGACAGCCACCCAAAGTCCTCATCTATGCTGCATCCAA
TCTAGAATCTGGGATCCCAGCCAGGTTTAGTGGCAGTGGGGCTGGGACAGACTTCAC
CCTCAGCATCCATCCTGTGGAGGAGGAGGATGCTGCAACCTATTACTGTCAGCAAAG
TTATGAGGATCCGTGGACGTTCGGTGGCGGGACCAAGCTGGAGCTGAAA (SEQ ID
NO: 484)
NP3_G12
GACATTGTGATGACCCAGTCTCCAGCTTCTTTGGCTGTGTCTCTAGGGCAGAGGGCC
ACCATCTCCTGCAAGGCCAGCCAAAGTGTTGATTATGATGGTGATAGTTATATGAAC
TGGTACCAACAGAAACCAGGACAGCCACCCAAACTCCTCATCTATGCTGCATCCAAT
CTAGAATCTGGGATCCCAGCCAGGTTTGGTGGCGGTGGGTCTGGGACAGACTTCACC
CTCAACATCCATCCTGTGGAGGAGGAGGATGCTGCAACCTATTACTGTCAGCAAAGT
AATGAGGATCCGTTCACGTTCGGAGGGGGGACCAAGCTGGAGCTGAAA (SEQ ID NO:
485)
NP3_H2
GAAAATGTTCTCACCCAGTCTCCAGCTTCTTTGGCTGTGTCTCTAGGGCAGAGGGCC
ACCATCTCCTGCAAGGCCAGTCAAAGTGTTGATTATGATGGTGATAGTTATATGAAT
TGGTACCAACAGAAACCAGGACAGCCACCCAAACTCCTCATCTATGCTGCATCCAGT
CTAGATTCTGGGATCCCAGCCAGGTTTGGTGGCAGTGGGTCTGGGACAGACTTCACC
CTCAACATCCATCCTGTGGAGGAGGAGGATGCTGCAACCTATTACTGTCAGCAAAGT
AATGAGGATCCGTGGACGTTCGGTGGAGGCACCAAGCTGGAAATCAAA (SEQ ID NO:
486)
NP3_H9
GATGTCCAGATAACCCAGTCTCCATCCTCCCTATCTGTATCTGTGGGAGAAACTGTC
ACCATCACATGTCGAGCAAGTGAGAATATTTACAGTAATTTAGCATGGTATCAGCAG
AAACAGGGAAAATCTCCTCAGCTCCTGGTCTATGCTGCAACAGACTTAGCAGATGGT
GTGCCATCAAGGTTCAGTGGCAGTGGATCAGGCACACATTATTCCCTCAAGATCAAC
AGCCTGCAGTCTGAAGATTTTGGGAGTTTTTACTGTCAACATTTTTGGGGTACTCCGT
GGACGTTCGGTGGAGGGACCAAGCTGGAAATCAAA (SEQ ID NO: 487)
NP3_H12
GATATTCTGATGACTCAGGCTGCACCCTCCCTATCTGTATCTGTGGGAGAAACTGTT
ACCATCACATGTCGAGCAAGTGAGAATATTTACAGTGATTTAGCATGGTATCAGCAT
AAAGAGGGAAAATCTCCTCAGCTCCTGGTCTATGCTGCAACAGACTTAGCAGATGGT
GTGCCATCAAGGTTCAGTGGCAGTGGATCAGGCACACAGTATTCCCTCAAGATCAAC
AGCCTGCAGTCTGAAGATTTTGGGAATTATTACTGTCAACATTTTTGGGGTACTCCGT
GGACGTTCGGTGGAGGGACCAAGCTGGAAATAAAA (SEQ ID NO: 488)

Although various embodiments of the disclosure have been described and illustrated, it will be apparent to those skilled in the art in light of the present description that numerous modifications and variations can be made. The scope of the invention is defined more particularly in the appended claims

1. An antibody composition comprising a heavy chain having at least 80%, or at least 85%, or at least 90%, or at least 95% sequence identity with any one of SEQ ID NOs: 1-48.

2. The antibody composition of claim 1, wherein the heavy chain comprises any one of SEQ ID NOs: 1-48.

3. An antibody composition comprising a light chain having at least 80%, or at least 85%, or at least 90%, or at least 95% sequence identity with any one of SEQ ID NOs: 49-96.

4. The antibody composition of claim 3, wherein the light chain comprises any one of SEQ ID NOs: 49-96.

5. An antibody composition comprising:
(a) a heavy chain having at least 80%, or at least 85%, or at least 90%, or at least 95% sequence identity with any one of SEQ ID NOs: 1-48; and
(b) a light chain having at least 80%, or at least 85%, or at least 90%, or at least 95% sequence identity with any one of SEQ ID NOs: 49-96.

6. The antibody composition of claim 5, wherein:
(a) the heavy chain comprises any one of SEQ ID NOs: 1-48; and
(b) the light chain comprises any one of SEQ ID NOs: 49-96.

7. A method of treating an individual infected with SARS-CoV-2, the method comprising administering to the individual an effective amount of an antibody composition according to claim 1.

8. A method of preventing SARS-CoV-2 infection in an individual, the method comprising administering to the individual an effective amount of an antibody composition according to claim 1.

9. The method of claim 7, wherein the antibody composition is administered together with an adjuvant.

10. A method of identifying SARS-CoV-2 in an individual, the method comprising:
(a) isolating a biological sample from the individual;
(b) incubating the biological sample with an antibody composition according to claim 1; and
(c) detecting a biological interaction between the biological sample and the antibody composition.

11. A kit for identifying SARS-CoV-2 in a biological sample, the kit comprising:
(a) an antibody composition according to claim 1; and
(b) instructions of use.

12. An enzyme-linked immunosorbent assay (ELISA) test kit comprising:
(a) an antibody composition according to claim 1; and
(b) instructions of use.

13. Use of an antibody composition according to claim 1 for treating SARS-CoV-2.

14. Use of an antibody composition according to claim 1 to formulate a medicament for treating SARS-CoV-2.
